(19) United States
(12) Patent Application Publication
(10) Pub. No.: US 2023/0124095 A1
(43) Pub. Date: Apr. 20, 2023

(54) AMORPHOUS CALCIUM CARBONATE FOR TREATMENT OF ACIDOSIS

(71) Applicant: AMORPHICAL LTD., Ness Ziona (IL)

(72) Inventors: Yossi BEN, Moshav Zofer (IL); Yigal BLUM, San Jose, CA (US); Yehudit NATAN, Tel Aviv (IL)

(21) Appl. No.: 17/909,549

(22) PCT Filed: Jan. 28, 2021
(86) PCT No.: PCT/IL2021/050103

§ 371 Date: Sep. 6, 2022

Related U.S. Application Data
(60) Provisional application No. 62/987,952, filed on Mar. 11, 2020.

(51) Int. Cl. A61K 33/10; A61K 9/00; A61K 31/728; A61P 29/00; A61P 35/00; A61P 11/00; A61P 31/14; A61K 9/16

(52) U.S. Cl. CPC A61K 33/10; A61K 9/0053; A61K 31/728; A61P 29/00; A61P 35/00; A61P 11/00; A61P 31/14; A61K 9/16

(57) ABSTRACT

The preset invention is directed to ACC particles stabilized by at least one stabilizing agent, a pharmaceutical composition including same, and methods of using same, such as for treating or preventing an acidosis-related disease or condition in a subject in need thereof.

### CROSS-REFERENCE TO RELATED APPLICATIONS

This application claims the benefit of priority of U.S. Provisional Patent Application No. 62/987,952, titled “CARBONATE OF DIVALENT METALS FOR TREATMENT OF ACIDOSIS”, filed Mar. 11, 2020, the contents of which are incorporated herein by reference in their entirety.

### FIELD OF THE INVENTION

The present invention relates to use of amorphous calcium carbonate for treatment of disease or conditions related and/or associated with acidosis, including, but not limited to e.g., viral infections and viral respiratory diseases.

### BACKGROUND

### Acidosis Conditions

Acidosis is a condition of increased acidity (lower pH) in the blood and other body tissues and fluids (i.e., an increased hydrogen ion concentration). It can be categorized as “systemic” or “local” and is caused by “metabolic” or “respiratory” condition or malfunctioning of organs in the body, such as kidneys. It can be acute or chronic, based on the origin of the body malfunctioning that cause the acidosis. In some cases, acidosis occurs when the body cannot generate enough bicarbonate, which serves as the main self-generated buffer of the body. In other cases, it is associated with the formation of lactate and therefore known as lactic acidosis, although the protons are generated separately of the lactate, formed by the metabolic pathway of glycolysis. In other cases, acidosis is due excessive use of energy, when adenosine triphosphate (ATP) is converted to adenosine diphosphate (ADP) while releasing a proton. In the absence of ATP regeneration, via metabolic pathways, ADP further degrades to adenosine monophosphate (AMP) while releasing another proton. Also, acidosis can be caused by the presence of excessive level of CO₂ in the blood. The excessive level of CO₂ is then converted to carbonic acid, via catalytic reaction of carbonic acid anhydrase.

The rate of cellular metabolic activity affects and, at the same time, is affected by the pH of the body fluids. There are two main types of acidosis: (1) Respiratory acidosis—resulting from a build-up of carbon dioxide in the blood (hypercapnia) due to hypoventilation, and or incapability to release the CO₂ at the rate of its formation due to extraneous/continuous body performance. In these situations, excessive levels of CO₂ in the body fluids reacts with water in the presence of carbonic anhydrase and forms carbonic acid. The carbonic acid dissociates into hydrogen ion and bicarbonate, hence acidifying the surrounding area or the blood system. (2) Metabolic acidosis—primarily associated with the reduction of bicarbonate (HCO₃⁻), typically with compensatory reduction in carbon dioxide partial pressure (Pco₂). The pH may be markedly low or slightly subnormal (the normal pH range is 7.35 to 7.45 in the bloodstream). One subtype of metabolic acidosis is known as “lactic acidosis”, which occurs due to altered lactate metabolism; for example, “Warburg Effect” (i.e., glycolysis) that occurs even at the presence of oxygen is a hallmark for cancer cells. The products of glycolysis (usually the preferred metabolic pathway in the absence of oxygen (hypoxia) are L-lactate and hydrogen ions.

### Examples of Acidosis

Inflammation, ischemia, and the microenvironment of tumors (solid and hematologic malignancies) are often accompanied by a reduction of extra-cellular pH (acidosis), due to a shift in cells metabolic pathway from oxidative phosphorylation to glycolysis. This acidosis phenomenon is derived from the metabolic and genetic changes (either mutations or changes in gene expressions) that cancer cells undergo. These changes cause a shift in cancer cell metabolism towards glycolysis, even in the presence of oxygen, called Warburg Effect or aerobic glycolysis. Consequently, an increase in extracellular pH occurs due to release of protons (hydrogen cations) and lactate or infusion of bicarbonate to quench the intracellular acidity. This stresses the healthy cells in the acidic environment and acts on cellular signaling and transcription pathways, which results in the release and enhanced activity of harming proteolytic enzymes, e.g., Cathepsins.

Many cancer types, almost all solid tumors, as well as hematological malignancies involve local acidosis in the tumor's microenvironment. The influence of the local acidosis in these diseases or pathological states results with worsening and advancing the diseases in the pathological state as well as inhibiting the immune system counter activity. For example, acidosis is the key mechanism for cancer cells' proliferation, invasion, and metastasis activities.

Modulating tumor acidosis has been shown to inhibit metastasis and raise the tumor's pH, either by using bicarbonate or a non-volatile buffer (aside the vast buffering activity of the bicarbonate, also basic phosphate anions and amino compounds participate in the neutralization and buffering of the pH).

In the context of non-cancer inflammations and associated pains, acidosis phenomenon is a key mechanism in pathological conditions (including chronic pain and abscesses) and is well recognized as a target for therapeutic development. Levels of pH were shown to be reduced in inflamed or tumor tissues of human and animals, including wounds, chronic diseases such as osteoporosis, rheumatoid arthritis, arteritis, diabetic wounds, abscesses, and extraneous muscle activities.

Most pathological conditions that cause local or systemic acidosis by overcoming the self-buffering activities of the body fluids overlap with those diseases that are characterized by glucose metabolic disorders, including diabetes mellitus, inflammation, and cancer.

Different pathological conditions may cause the imbalance of the buffering activities of bodily fluids.

A multitude potential causes of systemic and local acidosis exists. Local acidification may result from growth factors or cytokine stimulation of cell metabolism, vascular disease, ischemia, infection, tumors, or inflammation. In fracture healing, immediately after the initial trauma, fracture hematoma is characterized by hypoxia and low pH.

At a systemic level, in addition to renal and respiratory diseases, which affect the production or the elimination of bicarbonate, sources for excessive accumulation of H+ are anaerobic exercise, excessive energy consumption rates (resulting in the acidifying conversion of ATP to ADP and AMP), gastroenteritis, the excessive consumption of proteins or of other acidifying substances, anemias, acquired immunodeficiency syndrome (AIDS), aging, and menopause, and diabetes. Remarkably, most of the aforementioned conditions overlap with those, which occur with an altered glucose or insulin metabolism, suggesting a strong relationship between acidosis and insulin metabolism or insulin receptor signaling. One of the major functions of insulin is a stimulatory effect on glycolysis that occurs, during the increased level of circulating glucose. In turn, glycolysis promotes lactate and proton production. Hyperlactatemia is a recurrent clinical feature in diabetic patients.

It is known that acidification of the extracellular fluids stimulates the release of TGF-β in normal cells, since either in mesenchymal stem cells (MSC), proximal tubular cells, or hippocampal cultures, acidosis causes a four-fold increase of the levels of TGF-β or TGF-β1 expression and bioactivity. In turn, the systemic release of TGF-β modulates pancreatic development and islet homeostasis and inhibits the replication of β-cells. This information suggests that acid-induced systemic release of TGF-β may decrease the circulating level of insulin, thereby partially contributing to the feedback inhibitory mechanism of acidosis that ultimately results in insulin resistance. Interestingly, ACC has been found to downregulate the expression of TGFB1 (which encodes TGF-β1) in A549 (non-small cell lung cancer) cell line. TGF-β acts as a pro-tumor cytokine in cancer by promoting tumor angiogenesis, immune-escape, and metastasis.

Diabetic acidosis evolves when acidic substances, known as ketone bodies, build up in the body. This most often occurs with uncontrolled Type 1 diabetes. It is also referred to as diabetic ketoacidosis (DKA).

Hyperchloremic acidosis is another type of disease related to acidosis, which results from excessive loss of bicarbonate from the body due to diarrhea or renal conditions.

When exercising, skeletal muscle cells in healthy humans simultaneously produce and consume L-lactate. L-lactate extraction by overactive muscle cells is proportional to the arterial L-lactate concentration. Net L-lactate production is increased by muscle contractions and the released amount is balanced with the release of H+ ions from the cells, which are excessively formed by parallel energy consuming activities (consuming of ATP and increased CO₂ levels). Hence, a high-intensity exercise may cause both metabolic and systemic acidosis.

Another example of acidosis is the distal renal tubular acidosis (dRTA). Renal acid-base homeostasis is a complex process, effectuated by bicarbonate reabsorption and acid secretion. Impairment of urinary acidification is called renal tubular acidosis (RTA). Distal renal tubular acidosis (dRTA) is the most common form of RTA syndromes. The characteristic features of dRTA are the presence of systemic acidosis together with the inability to acidify the urine to a pH<5.3. dRTA is associated with many diseases each with their own pathophysiology. dRTA is associated with autoimmune diseases such as primary Sjögren syndrome and systemic lupus erythematosus. The most common symptoms of dRTA are nephrolithiasis and metabolic acidosis. Fatigue is a frequent complaint, possibly related to the metabolic acidosis-induced hyperventilation.

Patients with chronic metabolic acidosis are prone to develop osteoporosis. Metabolic acidosis affects bone by exchanging protons for sodium, potassium, calcium, carbonate, and phosphate. The acidic conditions also assist the hydrolysis of the Ca—O—P bonds that constitute the inorganic network of the bones. The continuous sequestration of protons in bone stimulates both osteoclast development and activity, while simultaneously inhibiting the activity of osteoblast cells. As a consequence, bone resorption increases, enhancing release of calcium and mineral buffers (phosphate) from the bone surface. Eventually, this mechanism leads to net bone loss and hypercalciuria.

Sepsis is a life-threatening condition that arises when the body's response to infection is imbalanced and causes injury to its tissues and organs. Increased blood lactate concentration (hyperlactatemia) and lactic acidosis (hyperlactatemia and serum pH<7.35) are common in patients with severe sepsis or septic shock and are associated with significant morbidity and mortality. Lactate is an important energy “shuttle” whose production is triggered by a variety of metabolites even before the onset of anaerobic metabolism as part of an adaptive response to a hypermetabolic state and, in particular, during sepsis.

In severe cases of acidosis, and systemic ones in particular, in which the blood pH is extremely low, an infusion of highly soluble sodium bicarbonate with or without sodium carbonate is administered to overcome the acidity.

### Cathepsins

Cysteine Cathepsins are lysosomal peptidases, involved on one hand in general intracellular protein degradation and, on the other, in the regulation of a number of specific physiological processes. Cathepsins were found to be involved in many diseases, such as osteoporosis, osteoarthritis, pycnodysostosis, rheumatoid arthritis, atherosclerosis, Down syndrome, Alzheimer's disease, and asthenic bulbar paralysis. Also, cysteine Cathepsins upregulation has been demonstrated in many human tumors, including breast, lung, brain, gastrointestinal, head and neck cancer, and melanoma. Aside cancer diseases, they have shown to participate in inflammatory diseases, such as inflammatory myopathies, rheumatoid arthritis, atherosclerosis, and periodontitis. Most of the Cathepsins are active in a slightly acidic environment as found in many pathological processes (or in the lysosomes under normal conditions). Whereas, under physiological pH they are instable. A decrease of the Cathepsin levels or activity may indicate that acidosis has decreased, i.e., the pH has elevated. An exception is Cathepsin S, which is active under physiological conditions and even under slightly alkaline conditions.

Cathepsin B is involved in tumor metastasis by degradation of extracellular matrix components. The activity and levels of Cathepsin B were found to be elevated at acidic pH. Cathepsin B is involved in tumors metastases of several types of cancers, such as: breast carcinomas, melanoma, gastric cancer, lung cancer, colon cancer, ovarian cancer, cervical cancer, and pancreatic carcinomas.

Another example is Cathepsin K (and also Cathepsin B) which were demonstrated to be closely connected with the joint destruction in human's Rheumatoid Arthritis (RA). Cathepsin K is expressed by osteoclasts and synovial fibroblasts and degrades key components of bone and cartilage. It was found that a Cathepsin K inhibitor given to a rheumatoid arthritis model in rats significantly reduced hind paw thickness and the arthritis score and prevented a decrease in bone mineral density. Cathepsin K inhibitors are also investigated as possible therapeutic target for osteoporosis and osteoarthritis.

### How Carbonate Works

Similar to cells and tissues in-vivo, in-vitro cell culture systems need to have a small range of basic pH, usually between 7.2-7.4 in order for mammalian cells to thrive and proliferate. This pH is commonly maintained by the presence of CO₂ surrounding the culturing environment (supplied into the incubator) and buffering reagents such as Hepes, sodium bicarbonate, and rely on CO₂/HCO₃⁻ buffer homeostasis for maintaining the slight basicity. Despite the pros and cons of each buffering system, the need for frequent medium replacement remains as the cells proliferate and their biomass increases.

### Limitations of Acidosis Treatment

The utility of bicarbonate administration to patients with severe metabolic acidosis remains controversial. Chronic bicarbonate replacement is obviously indicated in patients who continue to lose bicarbonate in an ambulatory setting, particularly patients with renal tubular acidosis syndromes or diarrhea. There were several attempts to treat acidosis in inflammations and tumors by employing the use of sodium bicarbonate. There were also attempts to address the products of acidosis for example, by inhibiting acidophilic Cathepsins. Sodium bicarbonate is the predominant buffer used in dialysis fluids and patients on maintenance dialysis are subjected to a load of sodium bicarbonate during the sessions, suffering a transient metabolic alkalosis of variable severity. Side effects associated with sodium bicarbonate therapy include hypercapnia, hypokalemia, ionized hypocalcemia, and QT interval prolongation. Furthermore, the use of bicarbonate is restricted due to its high alkalinity, high dose required, marginal effectivity if used as acute treatments, and inconclusive evidence for its effectiveness.

Sodium bicarbonate is also taken orally by athletes in order to improve performances, especially in sporting events involving high rates of anaerobic glycolysis, which are otherwise limited by the body's capacity to manage the progressive increase in intracellular acidity. However, it is limited due to side effects mainly gut discomfort.

The use of Cathepsins inhibitors is also limited since Cathepsins have essential roles in normal body functions and the effect of these inhibitors was associated with adverse effects and off target effects. Proteases targeted therapies have not been very successful. The ideal inhibitor would be a non-covalent, reversible inhibitor with excellent selectivity, good bioavailability, and no side effects. The major issues in limiting inhibitor-designed drugs are still bioavailability and toxicity.

### Osteoarthritis

Inflammatory joint diseases are a group of diseases in which joints in the body become infected, manifested by pain, swelling, redness, stiffness and/or decrease in mobility of the inflamed joints. Diagnosis of the inflammatory nature of the disease is frequently based upon this typical clinical presentation as well as upon radiographic examination and aspiration and examination of synovial joint fluid. Examination of joint fluid of an inflamed joint generally reveals elevation of various markers of inflammation, such as, leukocytes (including neutrophils), antibodies, cytokines, cell adhesion molecules, and complement activation products. Radiographic examination of affected joints generally reveals soft tissue swelling and/or erosive changes.

Osteoarthritis (also known as degenerative arthritis) and rheumatoid arthritis are two of the most common diseases involving joint inflammation. Osteoarthritis is a degenerative disease of the synovial joints, characterized by loss and erosion of articular cartilage, subchondral sclerosis, and bone overgrowth (osteophyte formation). Osteoarthritis can damage any joint in the body, but it most commonly affects joints in the hands, knees, hips, and spine. Rheumatoid arthritis is a systemic autoimmune disease characterized by the simultaneous inflammation of the synovium of multiple joints, leading to joint damage (e.g., destruction, deformation, and disability), and also damage to other organs in the body. Additional inflammatory joint diseases include, for example, gout (gouty arthritis), ankylosing spondylitis and psoriatic arthritis.

Treatment of inflammatory joint diseases aims at reducing pain, improving function, and minimizing damage to the joints. Osteoarthritis is typically treated with physical therapy and medications such as analgesics for pain relieve and non-steroidal anti-inflammatory drugs (NSAIDs), and also corticosteroids taken orally or injected directly into the joint in more severe cases. Additional treatments include injections of hyaluronic acid into the joints. Rheumatoid arthritis is typically treated with medications such as NSAIDs, corticosteroid medications and disease-modifying anti-rheumatic drugs (DMARDs), including biologic DMARDs, depending on the severity of the symptoms. While such treatments may be effective, long-term use of these medications is associated with various adverse effects, some of which may be severe.

It has been suggested that patients with osteoarthritis and rheumatoid arthritis should consume calcium supplements for improving bone density and reducing or preventing bone loss that is associated with these diseases, and which may be further enhanced due to treatment with corticosteroids.

For example, the effects of supplemental calcium (calcium carbonate (1,000 mg/d) and vitamin D3 (500 IU/d)) on bone mineral density of patients with rheumatoid arthritis, and the relation between the effects of this supplementation and corticosteroid use, was previously studied.

As another example, a food effective in bone's mineral density increase and joint healthcare, as well as its preparation method, were disclosed. The supplementation is composed of calcium carbonate, glucosamine potassium sulfate, chondroitin sulfate, casein phosphopeptide and vitamin D3. The bone mineral density increases, and joint healthcare food contains mineral matters, which can supplement human body calcium and the casein phosphopeptide and the vitamin D3 for promoting the calcium absorption and has good treatment and prevention effects on rheumatoid arthritis, degenerative arthritis, joint motion damage and bone joint aches. However, it has been shown that prolonged consumption of calcium supplements at large amounts is harmful, as the calcium can be incorporated into phosphate and oxalate precipitations in the body and clogs vain or damages tissues.

Others disclosed a biomimetic material based on energy-rich amorphous magnesium polyphosphate (Mg-polyP) microparticles that enhance cartilage synthesis and regeneration. One preferred formulation of the inventive material is a hyaluronic acid-Mg/Ca-polyP paste that can be produced from a water-soluble salt of polyP and water-soluble hyaluronic acid in the presence of water-insoluble/nearly insoluble calcium carbonate. The material through scavenging calcium ions (Mg²⁺/Ca²⁺ exchange) and binding of the calcium-polyP to hyaluronic acid shows biomechanical properties, comparable to cartilage and thus can be used for prevention of calcium crystal formation in the synovial fluid and treatment of joint dysfunctions caused by osteoarthritis.

Others provided formulations comprising amorphous or microcrystalline calcium carbonate and that the formulations are efficient in treating various pathological conditions, including proliferative diseases, neurological disorders, and musculoskeletal disorders. Also disclosed are orally administrable compositions comprising stable amorphous calcium carbonate (ACC). The compositions are prepared as solid dosage forms such as tablets, capsules, and powders, and are supplemented orally by subjects, such as for treating osteoporosis, osteomalacia and related diseases. Stabilized amorphous calcium carbonate (ACC) formulations, comprising ACC and a non-aqueous liquid carrier in which the ACC is dispersed were previously disclosed. Cosmetic and pharmaceutical compositions for use in treating or ameliorating a topical inflammation or a skin affliction comprising were previously disclosed. Compositions comprising amorphous calcium carbonate (ACC), suitable for administration by inhalation, buccal or sublingual administration and methods for their use in treating ACC-responsive diseases and conditions, were previously disclosed. Stabilized amorphous calcium carbonate (ACC) for treatment of several neurological, muscular and infertility diseases, was previously disclosed. Further use of ACC in methods of in vitro fertilization and improvement of sperm quality was described. Also, stabilized ACC was found to be useful for enhancing the growth of cell and tissue cultures, gametes, and embryos in vitro.

### Multiple Sclerosis

Multiple sclerosis is the most common immune-mediated disorder affecting the central nervous system. In the course of MS, an autoimmune response damages the myelin sheath. This damage disrupts the ability of parts of the nervous system to communicate, resulting in a wide range of signs and symptoms including changes in sensation, muscle weakness, movement and balance difficulties, speech and swallowing impairment, vision loss, fatigue etc. Relapsing-remitting form of the disease is diagnosed in 85% of MS patient. This form is characterized by symptoms lasting for several days to weeks and then disappearing partially or completely. The existing disease-modifying agents are only partially effective in preventing MS relapses, have a limited impact on the accrual of disability and have not been shown to be effective in progressive forms of the disease. The clinical response to the existing agents is at once suboptimal, heterogeneous, and unpredictable.

There is no known cure for multiple sclerosis. Treatments attempt to improve function after an attack and prevent new attacks. Medications used to treat MS, while modestly effective, can have side effects and be poorly tolerated.

Glatiramer acetate (also known as Copolymer 1, Cop-1, or Copaxone®) is an immunomodulator medication, currently used to treat multiple sclerosis. Glatiramer acetate is a mixture of random-sized peptides that are composed of the four amino acids found in myelin basic protein, namely glutamic acid, lysine, alanine, and tyrosine. Myelin basic protein is the antigen in the myelin sheaths of the neurons that stimulates an autoimmune reaction in people with MS, so the peptide may work as a decoy for the attacking immune cells. Glatiramer acetate is approved in the United States to reduce the frequency of relapses, but not for reducing the progression of disability. Observational studies, but not randomized controlled trials, suggest that it may reduce progression of disability. While a conclusive diagnosis of multiple sclerosis requires a history of two or more episodes of symptoms and signs, glatiramer acetate is approved to treat a first episode, anticipating a diagnosis. Copaxone is used to treat relapsing-remitting multiple sclerosis and administered by subcutaneous injection.

Specific studies demonstrated alkaline compounds as useful in preventing and treating inflammatory diseases. For example, in vitro studies demonstrated that sodium bicarbonate promotes the recovery of intracellular pH of chondrocytes leading to a normalization of intracellular metabolic activities.

It has been demonstrated in preclinical studies that amorphous calcium carbonate (ACC) has an enhanced solubility and bioavailability over crystalline calcium carbonate (CCC). This, in turn, is reflected by higher bone density upon administration. In addition, an osteoporosis model in rats ACC provided superior results not only compared to CCC but also compared to calcium citrate and Alendronate (bisphosphonate), which is currently the gold standard for osteoporosis treatment. The preclinical findings of higher bioavailability and intestinal absorption were corroborated by results of a clinical study performed with menopausal women.

Amorphous Calcium Carbonate (ACC) is the most thermodynamically unstable polymorph of CaCO3; it is sensitive to heat and moisture and converts to more metastable crystalline calcium carbonate polymorphs. Several methods were developed to stabilize ACC including development of novel methods of manufacturing, stabilizers, or coating the ACC particles.

Inflammation is part of the complex biological response of body tissues to harmful stimuli, such as pathogens, damaged cells, or irritants and is a protective response involving immune cells, blood vessels, and molecular mediators. The function of inflammation is to eliminate the initial cause of cell injury, clear out necrotic cells and tissues damaged from the original infection cause and the inflammatory process, and initiate tissue repair. The five classical signs of inflammation are heat, pain, redness, swelling, and loss of function. Inflammation can be classified as either acute or chronic.

Inflammatory bowel disease (IBD) is a group of inflammatory conditions of the colon and small intestine. Crohn's disease and ulcerative colitis (UC) are the principal types of inflammatory bowel disease. Many of the IBD patients suffer from calcium deficiencies, and specifically, between 22-55% of Crohn's disease, and 32-67% of ulcerative colitis patients have osteopenia. Symptoms of the diseases depend on extent and severity of inflammation. UC symptoms include bloody diarrhea, abdominal cramping, rectal tenesmus, i.e., fecal urgency, systemic symptoms such as fever, decreased stamina and weight loss. One third of the patients experience extra-intestinal manifestations. Crohn's disease symptoms include diarrhea, chronic abdominal pain, weight loss, fever, perianal disease, and extra-intestinal manifestations. The symptoms may vary with type and location of disease (structuring, fistulizing).

The connection between inflammation and tumorigenesis is well established and has received a great deal of supporting evidence from genetic, pharmacological, and epidemiological data. IBD is an important risk factor for the development of colon cancer. Inflammation is also likely to be involved with other forms of sporadic as well as heritable colon cancer. Currently acceptable treatment of IBD is mostly symptomatic and depends on the severity of the disease. Among different pharmacological treatments aminosalicylates, oral steroids and corticosteroids are the most common. Additional drugs used are immunomodulatory drugs, such as azathioprine (AZA) and 6-mercaptopurine (6-MP) and biologic therapy such as Infliximab or Adalimumab (monoclonal antibodies against TNF-α), cyclosporine (immunosuppressant drug) and surgery in severe cases.

Coronaviruses

Coronaviruses (CoVs) are the largest group of viruses belonging to the Nidovirales order, which includes Coronaviridae, Arteriviridae, and Roniviridae families. The Coronavirinae comprise one of two subfamilies in the Coronaviridae family, with the other being the Torovirinae. Coronaviruses are associated with illness from the common cold to more severe conditions such as Severe Acute Respiratory Syndrome (SARS-CoV) and Middle East Respiratory Syndrome (MERS-CoV). Severe acute respiratory syndrome coronavirus 2 (SARS-CoV-2) is the positive-sense, single-stranded RNA coronavirus that causes the coronavirus disease 2019 (COVID-19), responsible for the 2019-20 Wuhan coronavirus outbreak. Coronaviruses are zoonotic, meaning they are transmitted between animals and people. Common signs of infection include respiratory symptoms, fever, coughing, shortness of breath and breathing difficulties. High concentrations of cytokines were recorded in plasma of critically ill patients infected with 2019-nCoV. In more severe cases, infection can cause pneumonia, severe acute respiratory syndrome, kidney failure and even death.

There is still a great need for compositions and methods of using same, in the treatment and/or amelioration of acidosis-related diseases.

### SUMMARY

In some embodiments, the present invention is based, in part, on the findings that nanometric ACC particles allow neutralizing systemic and local acidosis in a controlled manner.

In sharp contrast to ACC, bicarbonate and bicarbonate/carbonate solutions fail to provide the desired therapeutic effect due to: (a) limited efficacy; (b) incapability to control the release and the initial pH at administration; (c) physical side effects, when taken orally, (d) decomposition of the bicarbonate and carbonate in the stomach (associated with high discomfort); (e) rapid removal from the blood system by counter-reactivity of the renal system and (f) undesired uptake of sodium.

The present invention is based, in part, on the findings that ACC nanoparticles can fuse and penetrate through mucous membranes into the blood system and absorbed into the blood vessels and body fluids in their solid particulate form and dissolve (or not dissolve) as a function of the local pH. Hence, they serve as “solid buffers”. The nanometric particles can be absorbed into blood vessels and body fluids via membranes of the intestine, mouth or lung walls or can be directly injected or infused in a suspension form.

In contrast to the herein disclosed compositions, the use of soluble bicarbonates and carbonates is much less effective if given either orally as a powder or by drinking, injection, or infusion of its solution to the blood, due to their high solubility and therefore, fast removal and clearance from the body, decomposition in the stomach, and dosage limitation due to high level of sodium. Hence, the bicarbonate treatment is not found to be very effective in ongoing chronic therapy. Further, in high solution concentrations of sodium bicarbonate, the pH is about 8.5 or higher, and therefore, is harmful to cells and blood vessels.

According to a first aspect, there is provided a method for treating a subject afflicted with an acidosis-related disease or condition, comprising orally administering to the subject a therapeutically effective amount of a solid composition comprising amorphous calcium carbonate (ACC) particles stabilized by at least one stabilizing agent, wherein the solid composition of ACC particles is formulated for controlled release.

According to another aspect, there is provided a method for treating a subject afflicted with an acidosis-related disease or condition, comprising administering to the subject a therapeutically effective amount of an aqueous composition in the form of a dispersion or suspension comprising ACC particles stabilized by at least one stabilizing agent, wherein the ACC particles are substantially uniformly dispersed or suspended in the composition, and wherein the administering is injecting.

According to another aspect, there is provided a method for treating a subject afflicted with inflammation or a disease or a condition associated therewith, the method comprising administering to the subject a therapeutically effective amount of: (i) a solid composition of ACC stabilized by at least one stabilizing agent; (ii) an aqueous composition in the form of a dispersion or suspension of ACC particles stabilized by at least one stabilizing agent; or (iii) a combination of (i) and (ii), thereby treating the subject afflicted with inflammation or a disease or a condition associated therewith.

According to another aspect, there is provided a combination comprising: ACC stabilized by at least one stabilizing agent; and glatiramer acetate or a pharmaceutically acceptable salt thereof, for use in the treatment of multiple sclerosis in a subject in need thereof.

In some embodiments, the ACC particles are agglomerated particles.

In some embodiments, the composition further comprises an enteric coating.

In some embodiments, the composition comprising ACC particles is coated or encapsulated within the enteric coating.

In some embodiments, injecting comprises: intravenously injecting, intraperitoneal injecting, locally injecting, or any combination thereof.

In some embodiments, the method further comprises diagnosing the acidosis-related disease or condition in the subject prior to the administering.

In some embodiments, the acidosis-related disease or condition is selected from the group consisting of: inflammation or a disease or condition associated therewith, prostate cancer, colorectal cancer, non-small cell lung cancer (NSCLC), human epidermal growth factor receptor (HER) positive breast cancer, and any combination thereof.

In some embodiments, the inflammation or a disease or condition associated therewith is related or derived from a physical activity.

In some embodiments, the inflammation or a disease or condition associated therewith being related or derived from a physical activity comprises: hip stress fracture, inflammation of the adductor magnus, swelling, redness and local warmness of the knee, or any combination thereof.

In some embodiments, the acidosis-related disease or condition is selected from the group consisting of: rheumatoid arthritis, diabetes mellitus, arteritis, osteoarthritis, hyperlactatemia, renal tubular acidosis, an infectious disease, ventilatory failure, sepsis, anaerobic and aerobic exercise, and any combination thereof.

In some embodiments, the acidosis-related disease or condition is rheumatoid arthritis.

In some embodiments, the administering is intraperitoneally injecting.

In some embodiments, the infectious disease is induced by a virus.

In some embodiments, the infectious disease is a respiratory disease.

In some embodiments, the virus belongs to a family selected from the group consisting of: Coronaviridae, Filoviridae, Arenaviridae, Orthomyxoviridae, Paramyxoviridae, Retroviridae, Togaviridae, and Flaviviridae.

In some embodiments, the infectious disease is induced by a coronavirus.

In some embodiments, the infectious disease is Coronavirus disease 2019 (COVID-2019).

In some embodiments, the acidosis-related disease or condition involves acidophilic Cathepsin activity.

In some embodiments, the acidophilic Cathepsin is selected from the group consisting of: B, K, A, G, C, F, H, L, O, V, W, X, D, E, and any combination thereof.

In some embodiments, the acidophilic Cathepsin is Cathepsin B, Cathepsin K, or both.

In some embodiments, treating comprises reducing activity of the acidophilic Cathepsin in the subject.

In some embodiments, the infectious disease comprises a viral infectious disease.

In some embodiments, administering comprises administering by inhalation, sublingual administering, or both.

In some embodiments, administering comprises multiple administrations.

In some embodiments, the multiple administering comprises daily multiple administering.

In some embodiments, the administration by inhalation comprises administering the aqueous composition in the form of a dispersion or suspension comprising the ACC particles stabilized by at least one stabilizing agent in a wt % ranging from 1 wt % to 2.0 wt % of the dispersion or suspension.

In some embodiments, the sublingual administration comprises administering the solid composition of ACC particles stabilized by at least one stabilizing agent comprising calcium in an amount ranging from 1,000 to 2,500 mg calcium per day, in the form of ACC.

In some embodiments, the solid composition of ACC stabilized by at least one stabilizing agent is formulated for controlled release.

In some embodiments, the solid composition comprises an enteric coating.

In some embodiments, the solid composition of ACC is coated or encapsulated within an enteric coating.

In some embodiments, at least 30% of the ACC particles comprise primary particles having a maximal size ranging from 10 to 500 nm.

In some embodiments, the ACC is substantially soluble in pH ranging from 6.0 to 7.5.

In some embodiments, the at least one stabilizing agent is selected from the group consisting of: organic acids, phosphorylated, phosphonated, sulfated or sulfonated organic compound, phosphoric or sulfuric esters and ethers of hydroxy carboxylic acids and polyols, glucose and its derivatives, polysaccharides, phosphorylated amino acids, bisphosphonates, polyphosphonates, organic polyphosphates, inorganic polyphosphates, hydroxyl bearing organic compounds and polyols, proteins, salt and derivatives thereof, magnesium or a salt thereof, and any combinations thereof.

In some embodiments, the composition further comprises an additional biomedically active agent.

In some embodiments, the additional biomedically active agent is suitable for the treatment or prevention of an acidosis-related disease or condition.

In some embodiments, the additional active agent is hyaluronic acid.

In some embodiments, the composition is a nutraceutical composition or a pharmaceutical composition.

In some embodiments, the nutraceutical composition comprises a food supplement or a medical food.

In some embodiments, the ACC stabilized by at least one stabilizing agent and the glatiramer acetate or a pharmaceutically acceptable salt thereof are administered sequentially or simultaneously.

In some embodiments, the ACC stabilized by at least one stabilizing agent and the glatiramer acetate or a pharmaceutically acceptable salt thereof are formulated as a separate dosage form or are co-formulated as a single dosage form.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF FIGURES

FIG. 1 includes a graph showing shows the effect of ACC and CCC suspensions, added to medium containing 10% serum. The pH of the serum solution was first acidified by adding lactic acid. Then, the effect of adding various calcium carbonate suspensions (added at excess, thus calcium carbonate solids are still present at the end of the experiments) was assessed. ACC elevated pH from 6.6 to 7.4 and 7.8 depending on the exact ACC formulation. CCC did not dissolve at pH 6.6, therefore did not change the pH.

FIG. 2 includes a graph showing the pH measurements of medium supplemented with 10% FBS. Arrows show events of addition of lactic acid and ACC suspensions (1% Ca with 15% of Citric acid as a stabilizer, after filtration, having a pH of 7.52). After an initial jump to about pH 7.1 the pH keeps going up slowly until reaching pH of 7.4.

FIG. 3 includes a graph showing 4T1 cells grown in mediums consisting of various calcium sources (ACC, CCC and CaCl₂), subjected to glycostress assay. Cells extracellular acidification rate (ECAR) was measured vs. time and under different conditions: Basal respiration, Oligomycin (Oligo) injection, FCCP and antimycin injections. Low ECAR of ACC treated cells demonstrated their lower glycolysis rates vs. CaCl₂ and CCC, indicating that the glycolysis path is suppressed when the cells are exposed to higher induced pH.

FIG. 4 includes a graph showing 4T1 cells grown in different media, subjected to mitostress assay. Cells O₂ consumption rate (OCR) was measured vs. time and under different conditions: Basal respiration, Oligomycin (Oligo) injection, FCCP and antimycin injections.

FIG. 5 includes a graph showing tumors volume growth in mice treated with 0.5% Ca in ACC versus saline. Each group consisted of 8 mice. Data is presented as mean±SEM.

FIG. 6 includes a graphs showing Cathepsin B activity measure form tumors taken on day 21 from mice treated with either ACC or saline (n=8). Data is presented as mean±SEM. Different letters represent statistical significance (p<0.05).

FIG. 7 includes a graph tumors volume growth in mice treated with 0.5% Ca in ACC, saline, Cisplatin and a combination of ACC and cisplatin. Each group consisted of 8 mice. Data is presented as mean±SEM.

FIG. 8 includes a graph showing clinical scores in model mice with induced multiple sclerosis (MS). The clinical scores represent the extent of paralysis which the animals exhibited during the course of the experiment in the following treatment groups: (1) Saline; (2) Copaxone; (3) ACC; and (4) Copaxone+ACC.

FIG. 9 includes a graph showing Cathepsin B activity measured in tumors harvested from mice receiving different treatments (ACC, Cisplatin or saline). Results are described as mean±SEM; different letters represent statistical significance (p<0.05).

FIG. 10 includes a graph showing Cathepsin S activity measured in tumors harvested from mice e receiving different treatments (ACC, Cisplatin or saline). Results are described as mean±SEM, different letters represent statistical significance (p<0.05).

FIG. 11 includes a graph showing Cathepsin B activity levels measured in spinal cords from mice with induced MS that were intraperitoneally (i.p) administered with ACC suspension compared with mice that were i.p injected with saline. Results are presented as mean±SEM.

FIG. 12 includes a graph showing Cathepsin B activity levels measured in joint from model rats with induced rheumatoid arthritis (RA). Rats were i.p administered with ACC suspension and compared with rats that were i.p injected with saline. Data is presented as mean±SEM. Different letters represent statistical significance (p<0.05).

FIG. 13 includes a graph showing Cathepsin K activity measured in joints from model rats with induced RA receiving different treatments. Data is presented as mean±SEM. Different letters represent statistical significance (p<0.05).

FIG. 14 includes a graph showing Cathepsin B activity measured in LNCaP cells subjected to different treatments. Data is presented as mean±SEM. Different letters represent statistical significance (P<0.05).

FIG. 15 includes a graph showing Cathepsin B activity measured in Lewis lung carcinoma (LLC) cells subjected to different treatments. Data is presented as mean±SEM. Different letters represent statistical significance (p<0.05).

FIG. 16 includes a graph showing Cathepsin B activity levels measured in NIH/3T3 cells subjected to different treatments. Data is presented as mean±SEM.

FIG. 17 includes a graph showing the change of pH vs. Time (minutes) for displaying 1 to 3 ACC (Density) tablets in 100 ml of 0.16N HCl; Starting at pH=0.83.

FIG. 18 includes a graph showing the change of pH vs. Time (minutes) for 1 to 2 ACC (Density) tablets in a solution of 100 ml of 0.032N HCl; Starting at pH=1.5.

FIG. 19 includes a transmission electron microscopy (TEM) micrograph showing ACC primary particles, indicating that such particles are smaller than 100 nm.

FIG. 20 includes a graph showing samples #1-6 dispersion results. Specific content of each sample are described in Table 21. In these samples it can be seen that the second milling with the hammer mill showed better dispersion results compared to the same formulas that were milled with the rotary mill. This was expected due to the smaller sieve opening size. Even though some results showed good dispersion, none of the capsules reached the target weight (about 70% of the desired 200 mg Calcium dose).

FIG. 21 includes a graph showing includes a graph showing samples #7-10 dispersion results. Specific content of each sample are described in Table 21. The samples were milled with a larger opening size in the hammer mill, in order to produce larger granules and higher bulk density (compared to samples 1-6 of FIG. 20). Each sample was formulated with a different type of super-disintegrant. It can be seen that sample #7 had the best results (CCS). The filling weight was also very close to the target (90%-100%).

FIG. 22 includes a graph showing includes a graph showing samples #11-14 dispersion results. Specific content of each sample are described in Table 21. The amount of excipients was reduced in order to reach the target weight more consistently, compared to the previous samples (samples 1-10 of FIGS. 20 and 21). It can be seen that the dispersion was impacted due to the reduction of CCS and Avicel in the Formula. The weight target was reached in all samples.

FIGS. 23A-23E include graphs and chemical exchange saturation transfer (CEST) MRI micrographs showing that ACC treatment reduces tumors' volume and increases pH in the tumor area towards basic pH. (23A) A graph showing tumors' volumes measured since treatment beginning on day 11 of the study. Mice received either ACC or saline injected IP twice a day for 14 consecutive days. (23B) A vertical bar graph showing the CEST result before and after contrast agent (Iopamidol) injection to a mouse which was treated with ACC. Black bars indicate data acquired prior injection and white bars indicate post injection of contrast agent. (23C) MR image after injection of the contrast agent. White stains indicate basic pH. Arrows point to intra-tumoral basic pH regions; T—tumor. (23D) Vertical bar graph showing the CEST result before and after contrast agent (Iopamidol) injection to a mouse, which was treated with saline. Black bars indicate data acquired prior injection and white bars indicate post injection of contrast agent. (23E) MR image after injection of the contrast agent to a mouse. White stains indicate basic pH. No intra-tumoral basic pH regions were observed. T—tumor.

FIG. 24 includes a graph showing tumor growth rates of the different groups that were administered via IP or IV with either ACC-triphosphate (TP) or ACC-citric acid (CA). Data is presented as the mean±SEM.

### DETAILED DESCRIPTION

### Methods of Treatment and Acidosis-Related Diseases

According to some embodiments, there is provided a method for increasing local or systemic pH in a subject in need thereof comprising administering to the subject a therapeutically effective amount of amorphous calcium carbonate (ACC) or a pharmaceutical composition comprising same. In some embodiments, administering comprises orally administering. In some embodiments, the ACC or the pharmaceutical composition comprising ACC is formulated for pH controlled or delayed release of the ACC.

As used herein, the term “acidosis” refers to a condition in which pH values of body fluid is lower than the normal physiological pH range. Under normal physiological conditions, the pH of plasma and of most tissues is maintained at values slightly above neutral pH, in a very narrow range of pH values. In humans, the physiologically normal pH of blood/plasma and of most tissues is from approximately 7.35 to 7.45. Thus, local, or systemic acidity below pH 7.35 is considered acidosis. According to some embodiments, acidosis comprises systemic acidosis, e.g., the pH of blood or plasma is below 7.35. According to other embodiments, acidosis comprises local acidosis, e.g., in a particular area or tissue.

In some embodiments, acidosis comprises local acidosis, systemic acidosis, or both.

As used herein, the term “acidosis-related disease or condition” refers to any disease or condition wherein acidic pH is involved, propagates, induces, enhances, increases, necessary, required, any equivalent thereof, or any combination thereof, to the pathogenesis, pathophysiology, or both, of the disease or condition.

According to some embodiments, treating or preventing an acidosis-related disease or condition comprises inhibiting, reducing, blocking, lowering, decreasing, downregulating, any equivalent thereof, or any combination thereof, the expression level a gene or a plurality of genes involved in, inducing, propagating, increasing, enhancing, or any combination thereof, an acid environment, inflammation, tissue damage, unregulated or dysregulated cell proliferation, and any combination thereof. In some embodiments, treating or preventing an acidosis-related disease or condition comprises inhibiting, reducing, blocking, lowering, decreasing, downregulating, any equivalent thereof, or any combination thereof, the expression level a gene or a plurality of genes encoding a protein product, or a plurality thereof, having or characterized by having an acidophilic activity.

According to some embodiments, treating or preventing an acidosis-related disease or condition comprises increasing, enhancing, activating, promoting, upregulating, any equivalent thereof, or any combination thereof, the expression level a gene or a plurality of genes inhibiting, reducing, blocking, lowering, decreasing, or any combination thereof, an acid environment, inflammation, tissue damage, unregulated or dysregulated cell proliferation, and any combination thereof. In some embodiments, treating or preventing an acidosis-related disease or condition comprises increasing, enhancing, activating, promoting, upregulating, any equivalent thereof, or any combination thereof, the expression level a gene or a plurality of genes encoding a protein product, or a plurality thereof having or characterized by having a basophilic activity.

According to some embodiments, increasing local pH refers to pH of the blood system and/or interstitial fluid. Thus, according to some embodiments, the present invention provides a method of increasing interstitial and/or circulatory pH.

As used herein, the term “interstitial pH” refers to pH of an interstitial fluid.

As used herein, the term “interstitial fluid” refers to fluid surrounding cells in a tissue.

As used herein, the term “circulatory” refers to the blood system, and encompasses any fluid flowing and/or passing in or through a blood vessel.

According to some embodiments, there is provided a method for treating a subject afflicted with an acidosis-related disease or condition, comprising orally administering to the subject a therapeutically effective amount of a solid composition comprising amorphous calcium carbonate (ACC) particles stabilized by at least one stabilizing agent.

In some embodiments, orally comprises providing an oral enteric composition, as described herein. In some embodiments, orally refers to the providing or administering of a composition as described herein through the oral cavity, and wherein the active agent is absorbed in the gastrointestinal tract, and preferably, the intestine.

In some embodiments, the solid composition of ACC particles is formulated for controlled release.

As used herein, the term “pH-controlled release” refers to the release of the active agents as a function of the environmental pH.

As used herein, the term “delayed release” refers to a dosage form that releases a discrete portion or portions of drug at a time or at times other than promptly after administration, although one portion may be released promptly after administration. Enteric-coated dosage forms are common delayed-release products.

As used herein, the term “controlled-release” is defined as the release of an active agent at such a rate that concentrations of the agent at a target site or an intermediate, such as, but not limited to blood are maintained within the therapeutic range but below toxic concentrations over a period of time of at least 1 hr, at least 3 hr, at least 5 hr, at least 8 hr, at least 12 hr, at least 18, at least 24 hr, or any value and range therebetween. Each possibility represents a separate embodiment of the invention. In some embodiments, the controlled release is over a period of 1 to 24 hr, 3 to 20 hr, 6 to 18 hr, 10 to 22 hr, 4 to 12 hr, or 8 to 28 hr. Each possibility represents a separate embodiment of the invention.

In some embodiments, controlled release comprises or is delayed release.

According to some embodiments, the solid composition of ACC particles is administered enterally, such as orally. According to some embodiments, enteral administration comprises a delayed or controlled release administration. In some embodiments, delayed or controlled release is derived or corresponds to the pH of the environment. According to some embodiments, the solid composition comprising ACC particles is formulated as a delayed or controlled release formulation. In some embodiments, the plurality of ACC particles of the solid composition are collectively coated in an enteric coat or layer, or any equivalent thereof, configured to delayed or controlled release. According to some embodiments, the delayed release composition is an enteric coated composition. According to some embodiments, the solid composition comprises ACC particles coated with and/or encapsulated within an enteric coating. According to some embodiments, the enteric coating is an enteric coated capsule. According to some embodiments, the ACC particles are in the form of a powder, pellets, or granules.

The term “delayed release” as used herein refers to a composition formulated to release discrete portion or portions of drug at a time other than promptly after administration, for examples after passing a particular part of the gastrointestinal tract, e.g., after passing the stomach. In some embodiments, a solid composition comprising ACC particles as disclosed herein, is formulated for GI absorption. In some embodiments, a solid composition comprising ACC particles as disclosed herein, is formulated for passing through the stomach of a subject without being absorbed therein.

According to some embodiments, the ACC particles are released from the delayed release composition at pH above 6. According to some embodiments, the ACC particles are released from the delayed release composition at pH above 6.5. According to some embodiments, the ACC particles are released from the delayed release composition at pH above 7.0.

According to some embodiments, there is provided a method for treating a subject afflicted with an acidosis-related disease or condition, comprising administering to the subject a therapeutically effective amount of an aqueous composition in the form of a dispersion or suspension comprising ACC particles stabilized by at least one stabilizing agent, wherein the ACC particles are substantially uniformly dispersed or suspended in the composition, and wherein the administering is injecting.

In some embodiments, injecting comprises intravenously injecting, peritoneally injecting, locally injecting, catherization, or any combination thereof.

As used herein, the term “locally injecting” refers to administration directly to the targeted site. In some embodiments, locally administering refers to administration directly to the site in need of deacidification. In some embodiments, locally administering refers to administration directly to the site in need of pH elevation.

In some embodiments, the method further comprises a step of selecting or diagnosing acidosis-related disease or condition in the subject prior to the administering. In some embodiments, a subject diagnosed with acidosis-related disease is suitable for treatment according to the herein disclosed method.

Methods for diagnosing acidosis and/or acidosis-related disease in a subject are common and would be apparent to one of ordinary skill in the art. Non-limiting examples for such methods of determination include, but are not limited to, blood tests, arterial blood gas (e.g., levels of oxygen and carbon dioxide in the blood), blood pH, basic metabolic panel (e.g., examines kidney functioning and pH balance), calcium, protein, blood sugar, and electrolyte levels, chest X-ray, a pulmonary function test, a urine sample test, and others.

According to some embodiments, there is provided a method for treating a subject afflicted with acidosis-related disease or condition, comprising administering to the subject a therapeutically effective amount of a pharmaceutical composition comprising ACC particles stabilized by at least one stabilizing agent, and having size ranging from 10 to 1,000 nm.

In some embodiments, the acidosis-related disease or condition is selected from: inflammation or a disease or condition associated therewith, prostate cancer, intestinal cancer, non-small cell lung cancer (NSCLC), human epidermal growth factor receptor (HER) positive breast cancer, cervix cancer, fibroblastoa, or any combination thereof.

In some embodiments, treating a cancer, as disclosed herein, comprises inducing or achieving a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to lifestyle, chronic inflammation and so forth) to develop cancer. As used herein, “prevention of cancer” is thus to be understood to include prevention of metastases, for example after surgical procedures or after chemotherapy.

In some embodiments, treating cancer or any of the herein disclosed actions targeting a tumor, are directed to a solid tumor.

In some embodiments, treating a cancer, as disclosed herein, comprises inducing or achieving a decrease in a solid tumor size; a decrease in rate of solid tumor growth; stasis of a solid tumor size; a decrease in the number of metastasis; a decrease in the number of additional metastasis; a decrease in invasiveness of the cancer; a decrease in the rate of progression of the solid tumor from one stage to the next; inhibition of a solid tumor growth in a tissue of a mammal having a malignant cancer; control of establishment of metastases; inhibition of tumor metastases formation; regression of established solid tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to lifestyle, chronic inflammation and so forth) to develop cancer. As used herein, “prevention of cancer” is thus to be understood to include prevention of metastases, for example after surgical procedures or after chemotherapy.

In some embodiments, the composition comprising ACC particles stabilized by at least one stabilizing agent is suitable for treatment of cancer involving a solid tumor.

According to some embodiments, intestinal cancer comprises cancer of small intestine. According to one embodiment, small intestine cancer is selected from: adenocarcinoma, sarcoma, carcinoid tumor, gastrointestinal stromal tumor, or lymphoma.

According to some embodiments, intestinal cancer comprises a large intestine (bowel) cancer. According to one embodiment, bowel cancer is selected from: colon cancer, rectal cancer, or colorectal cancer.

In some embodiments, the acidosis-related disease or condition is selected from: rheumatoid arthritis, diabetes mellitus, arteritis, osteoarthritis, hyperlactatemia, renal tubular acidosis, an infectious disease, ventilatory failure, sepsis, anaerobic and aerobic exercise, and or combination thereof.

In some embodiments, the infectious disease is induced by a virus.

In some embodiments, the infectious disease is a respiratory disease.

As used herein, the term “respiratory disease” refers to any disease of the respiratory tract. As used herein, the term “respiratory tract” refers to the system that is involved with respiration or breathing. The respiratory tract is often divided into three segments, namely the upper respiratory tract (i.e., nose, nasal passages, paranasal sinuses, throat/pharynx), the respiratory airways (i.e., larynx, trachea, bronchi, and bronchioles), and the lower respiratory tract (i.e., the lungs, comprised of respiratory bronchioles, alveolar ducts, alveolar sacs, and alveoli).

According to some embodiments, the respiratory disease is a viral respiratory infection or a disease. In some embodiments, viral respiratory infection comprises common respiratory infection in an adult, a child, or both. According to some embodiments, the viral respiratory disease comprises a primary respiratory disease, a secondary bacterial infection, or both. According to some embodiments, the respiratory disease is induced by, caused by, results from, involves, or any combination thereof, a coronavirus.

The method of the invention, according to some embodiments, comprises treating or preventing an acidosis-related disease or condition comprising a respiratory infection. In some embodiments, the method comprises treating or preventing a respiratory infection in a subject in need thereof by administering to the subject a therapeutically effective amount of a stable or stabilized ACC or a pharmaceutical composition comprising same. According to one embodiments, the ACC is stabilized by at least one stabilizing agent. According to some embodiments, the method comprises administering the ACC via oral administration, parenteral administration, or a combination thereof. According to some embodiments, parenteral administration comprises oromusocal, inhalation, topical, local, and intravenous administration. According to some embodiments, oral administration or oromucosal is delayed release administration, as defined herein above. In some embodiments, oral administration as used herein does not refer to gingival or sublingual administration.

In some embodiments, the acidosis-related disease or condition comprises a viral disease. In some embodiments, the acidosis-related disease or condition involves any virus known to induce, utilize, increase, propagate, require, any equivalent thereof, or any combination thereof, acidic environment so as to exert its activity, e.g., binding to a host cell receptor, internalize into a host cell, evade any host intracellular anti-viral mechanism, genome duplication, viral particle assembly including genome packaging, induce host cell lysis, or any combination thereof. In some embodiments, the acidosis-related disease or condition is or comprises respiratory disease. In some embodiments, the respiratory disease is or comprises a viral respiratory disease. In some embodiments, the respiratory disease is induced by a coronavirus.

In some embodiments, the virus belongs to a family selected from: Coronaviridae, Filoviridae, Arenaviridae, Orthomyxoviridae, Paramyxoviridae, Retroviridae, Togaviridae, and Flaviviridae.

In some embodiments, the infectious disease is induced by a coronavirus.

In some embodiments, the infectious disease is Coronavirus disease 2019 (COVID-2019). In some embodiments, the respiratory disease is COVID-2019.

Severe acute respiratory syndrome coronavirus 2 (SARS-CoV-2), formerly known as the 2019 novel coronavirus (2019-nCoV), is a positive-sense single-stranded RNA virus. It is contagious among humans and is the cause of coronavirus disease 2019 (COVID-19). SARS-CoV-2 has strong genetic similarity to known bat coronaviruses, making a zoonotic origin in bats likely, although an intermediate reservoir such as a pangolin is thought to be involved. From a taxonomic perspective SARS-CoV-2 is classified as a strain of the species severe acute respiratory syndrome-related coronavirus. SARS-CoV-2 is the cause of the ongoing 2019-20 coronavirus outbreak, a Public Health Emergency of International Concern that originated in Wuhan, China. Because of this connection, the virus is sometimes referred to informally, among other nicknames, as the “Wuhan coronavirus”.

According to some embodiments, the coronavirus is a human coronavirus selected from the: Human coronavirus 229E (HCoV-229E), Human coronavirus OC43 (HCoV-OC43), Severe acute respiratory syndrome coronavirus (SARS-CoV), Human coronavirus NL63 (HCoV-NL63, New Haven coronavirus), Human coronavirus HKU1, Middle East respiratory syndrome-related coronavirus (MERS-CoV), and Severe acute respiratory syndrome coronavirus 2 (SARS-CoV-2).

In some embodiments, the virus is SARS-CoV-2 (COVID-2019).

In some embodiments, the method comprises treating an infectious disease or an inflammation derived therefrom, such as, but not limited to COVID-19, wherein a SARS-CoV-2 infection induces a dysregulated inflammation, i.e., cytokine storm. In some embodiments, a method for treating an infectious disease induced by a virus, e.g., COVID-19, comprises administering a composition comprising ACC particles, as disclosed herein, wherein the administering is by inhalation, sublingual administering, or both.

In some embodiments, administration by inhalation, such as, but not limited to for treating COVID-19 comprises administering the aqueous composition in the form of a dispersion or suspension comprising the ACC particles stabilized by at least one stabilizing agent in a wt % ranging from 0.1 wt % to 2.0 wt %, 0.1 wt % to 1.0 wt %, 0.3 wt % to 2.0 wt %, 0.7 wt % to 1.9 wt %, or 0.5 wt % to 2.2 wt %, of the dispersion or suspension. Each possibility represents a separate embodiment of the invention.

In some embodiments, sublingual administration, such as, but not limited to, treating COVID-19, comprises administering the solid composition of ACC particles stabilized by at least one stabilizing agent comprising calcium in an amount ranging from 1,000 to 2,500 mg, 1,000 to 2,000 mg, 8,000 to 2,200 mg, or 500 to 1,500 mg, calcium per day, in the form of ACC. Each possibility represents a separate embodiment of the invention.

According to some embodiments, the disease is COVID-19, and the present invention provides a method of treating COVID-19 comprising administering ACC particles as disclosed herein, to subject in need thereof, via inhalation and/or sublingually and/or orally.

According to some embodiments, the method comprises increasing the pH in the subject, thereby reducing the multiplicity of infection (MOI) of a virus, e.g., SARS-CoV-2, in the subject. In some embodiments, the method comprises increasing the pH in the subject, thereby reducing the rate of viral internalization into a host cell of the subject. In some embodiments, the method comprises increasing the pH in the subject, thereby reducing the binding rate, binding affinity, or both, of the virus or a protein thereof, e.g., a spike protein, to a host cell or a protein thereof, e.g., Angiotensin-converting enzyme 2 (ACE2 receptor). In some embodiments, the cell is an epithelial cell. In some embodiments, the cell is a respiratory epithelial cell. In some embodiments, increasing the pH comprises increasing the pH systemically, locally, or both. In some embodiments, treating according to the herein disclosed method comprises increasing the pH in a subject in need thereof.

According to some embodiments, the method comprises co-administration of other antiviral agents.

In some embodiments, administering comprises multiple administrations. In some embodiments, the multiple administering comprises daily multiple administering.

In some embodiments, the acidosis-related disease or condition is rheumatoid arthritis.

In some embodiments, there is provided a method for treating a subject afflicted with arthritis, such as rheumatoid arthritis, comprising intraperitoneally administering to the subject a therapeutically effective amount of an aqueous composition in the form of a dispersion or suspension of ACC particles stabilized by at least one stabilizing agent, thereby treating the subject afflicted with arthritis.

According to some embodiments, the method comprises modulating or affecting Cathepsin activity in a subject in need thereof.

In some embodiments, modulating or affecting comprises inhibiting, reducing, blocking, lowering, decreasing, any equivalent thereof, or any combination thereof, the activity of a Cathepsin. In some embodiments, Cathepsin comprises and acidophilic Cathepsin. In some embodiments, Cathepsin comprises a plurality of Cathepsins. According to some embodiment, the present invention provides a method of reducing activity of acidophilic Cathepsins in a subject in need thereof comprising administering to the subject an effective amount of ACC as disclosed herein, thereby reducing the activity of acidophilic Cathepsins. According to one embodiment, the ACC is stabilized by at least one stabilizing agent.

In some embodiments, the acidosis-related disease or condition involves acidophilic Cathepsin activity.

In some embodiments, the acidophilic Cathepsin is selected from: B, K, A, G, C, F, H, L, O, V, W, X, D, E, and any combination thereof.

In some embodiments, the acidophilic Cathepsin comprises or is Cathepsin B, Cathepsin K, or both.

In some embodiments, treating comprises reducing activity of the acidophilic Cathepsin in the subject.

As used herein, the term “Cathepsin” refers to a protein belonging to a group of lysosomal proteases that have a key role in cellular protein turnover. As used herein, the term “Cathepsin” encompasses serine proteases, aspartic proteases, and cysteine proteases. Based on their catalytic mechanism, Cathepsins are subdivided into serine (A and G), cysteine (B, C, F, H, K, L, O, S, V, W and X) and aspartic proteases (D and E). Most of the Cathepsins are acidophilic Cathepsins, e.g., are most active in a slightly acidic to acidic environment. An exception is Cathepsin S which is a non-acidophilic Cathepsin being active under physiological conditions and even under slightly alkaline conditions. According to some embodiments, the Cathepsins are human Cathepsins.

According to some embodiments, the method comprises reducing activity of Cathepsin B. According to other embodiment, the method comprises reducing activity of Cathepsin K. According to another embodiments, the method comprises reducing activity of Cathepsin B and K.

According to some embodiments, there is provided a method for treating a subject afflicted with inflammation or a disease or a condition associated therewith, the method comprising administering to the subject a therapeutically effective amount of: (i) a solid composition of ACC stabilized by at least one stabilizing agent; (ii) an aqueous composition in the form of a dispersion or suspension of ACC particles stabilized by at least one stabilizing agent; or (iii) a combination of (i) and (ii), thereby treating the subject afflicted with inflammation or a disease or a condition associated therewith.

In some embodiments, the inflammation or a disease or condition associated therewith is related or derived from a physical activity.

In some embodiments, the inflammation or a disease or condition associated therewith being related or derived from a physical activity comprises: hip stress fracture, inflammation of the adductor magnus, swelling, redness and local warmness of the knee, or any combination thereof.

According to some embodiments, the method comprises reducing inflammation of the joints in a subject with an inflammatory joint disease. In some embodiments, the method comprises administering to the subject a composition comprising ACC particles stabilized by at least on stabilizing agent, wherein the administering comprises via parenteral administration. According to some embodiments, the method comprises administering to the subject a composition comprising ACC particles stabilized by at least on stabilizing agent, wherein the administering is via enteral administration, parenteral administration, or both. In some embodiments, the inflammatory joint disease is osteoarthritis. In some embodiments, the inflammatory joint disease is rheumatoid arthritis. In some embodiments, the parenteral administration is parenteral systemic administration. In some embodiments, the administering is via a route of administration selected from: intravenous, intraperitoneal, intramuscular, subcutaneous, sublingual, buccal and inhalation administration. Each possibility represents a separate embodiment of the invention.

According to some embodiments, the administration is a short-term administration, e.g., administration for 1, 2, 3, 5, or 7 days. According to some embodiments, the administration is for 1, 2, 3 or 4 weeks. According to some embodiments, the administration is for a long term such as for 1, 2, 3, 4, 5, 6, 7, 8, 9 10, 9, 11 or 12 months. According to some embodiments, the treatment is a chronic treatment, for one or more years such as 2, 3, 4, 5, or more years.

In some embodiments, parenteral administration a route of administration selected from: intravenous, intraperitoneal, intramuscular, subcutaneous, locally injected, buccal and inhalation administration.

In some embodiments, inflammation excludes psoriasis. In some embodiments, inflammation excludes inflammation of the skin. In some embodiments, inflammation excludes an auto-immune reaction.

As used herein, the terms “treatment” or “treating” of a disease, disorder or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, alleviated symptoms of the disease, disorder or condition include reduced cell viability, induced cell apoptosis, inhibited cell proliferation, or a combination thereof.

In some embodiments, the method comprises administering a dose of 50 mg/day to 200 mg/day, 200 mg/day to 10,000 mg/day, 250 mg/day to about 9,000 mg/day, 500 mg/day to about 8,000 mg/day, 750 mg/day to 6,000 mg/day of ACC, 1,000 mg/day to 5,000 mg/day, or 1,500 mg/day to 4,000 mg/day of ACC particles stabilized with at least one stabilizing agent.

In some embodiments, the dose is per kg of a subject. In some embodiments, the dose refers to the amount of elemental calcium in the ACC stabilized by the at least one stabilizing agent.

According to some embodiments, the administration of ACC stabilized by at least one stabilizing agent comprises administering less than 20 mg/kg/day, less than 30 mg/kg/day, less than 50 mg/kg/day, less than 100 mg/kg/day, less than 150 mg/kg/day, or less than 200 mg/kg/day dose of ACC stabilized by the at least one stabilizing agent, or any value and range therebetween. Each possibility represents a separate embodiment of the invention. According to some embodiment, the administration comprises administering 5 to 200, 10 to 150, 15 to 120, 20 to 80, 30 to 70, or 40 to 60 mg/kg/day of ACC stabilized by the at least one stabilizing agent. Each possibility represents a separate embodiment of the invention. According to some embodiments, the administration comprises administering 0.1 to 30, 0.2 to 28, 0.3 to 26, 0.5 to 24, 1 to 22, 2 to 20, 3 to 18, 3 to 16, 4 to 15, 5 to 14, 6 to 12, or 8 to 10 mg/kg/day of ACC stabilized by the at least one stabilizing agent. Each possibility represents a separate embodiment of the invention. According to another embodiment, the administration comprises administering 100 to 15,000 mg/day, 200 to 12,000, 400 to 10,000 mg/day, or 600 to 8,000 mg/day of ACC stabilized by the at least one stabilizing agent. Each possibility represents a separate embodiment of the invention.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, “treating” comprises ameliorating and/or preventing.

As used herein, the term “therapeutically effective amount” of a drug or agent is an amount of a drug or an agent that, when administered to a subject will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentation. The therapeutically effective amount may be administered in one or more different types of administration.

In some embodiments, the composition further comprises an additional biomedically active agent.

In some embodiments, the additional biomedically active agent is suitable for the treatment or prevention of an acidosis-related disease or condition. In some embodiments, the additional biomedically active agent is suitable for the treatment or prevention of a joint inflammatory disease.

As used herein, the term “inflammatory joint disease” refers to any disease involving joint inflammation. Joint inflammation is typically characterized by pain, swelling, redness, stiffness and/or decrease in mobility of the joint. In some embodiments, the inflammatory joint disease is osteoarthritis. In other embodiments, the inflammatory joint disease is rheumatoid arthritis.

The terms “reduction of joint inflammation” or “reducing joint inflammation” encompass reduction of clinical manifestations characteristics of joint inflammation, such as, but not limited to, pain, swelling, redness and/or stiffness and decrease in mobility of the joint. Reducing joint inflammation also encompasses reducing markers of inflammation upon examination of joint fluid. Reducing joint inflammation may also be evaluated by observing reduction in soft tissue swelling and/or erosive changes by radiographic examination of joints.

In some embodiments, a subject diagnosed with an inflammatory joint disease exhibits symptoms of an inflammatory joint disease, and/or at risk of an inflammatory joint disease, for example, due to a genetic predisposition, age and/or physical injury.

It is to be understood that “reducing” refers to improvement with respect to the level of inflammation before the treatment according to the present invention.

In some embodiments, the additional biomedically active agent is hyaluronic acid.

In some embodiments, composition comprises effective doses of ACC and hyaluronic acid in a single dosage form. In some embodiments, the hyaluronic acid serves as a stabilizer of the ACC. According to some embodiments, further stabilizers of ACC are optional. In some embodiments, the hyaluronic acid serves as a carrier for ACC that is stabilized by one or more stabilizers. It is now disclosed that such formulations are advantageous for effective delivery and action of both the ACC and the hyaluronic acid. According to some embodiments the formulations are formulated for intraarticular injection.

For preparing a formulation in which the hyaluronic acid serves as a carrier of ACC that is stabilized by one or more stabilizers, a suspension of ACC stabilized by at least one stabilizer as described herein may be mixed with a hyaluronic acid composition. In some embodiments, a suspension of ACC 0.3% (w/v) of Ca elementary stabilized by at least one stabilizer as described above (e.g., by 10% triphosphate and 1% citric acid) is mixed with an effective dose of 0.0075 to 0.15% (w/v) of hyaluronic acid suspended in a water-based solution, preferably at pH 6.8.

For preparing a formulation in which the hyaluronic acid serves as a stabilizer of ACC, a water-based suspension is prepared, of ACC 0.3% (w/v) Ca elementary stabilized by 1-20% hyaluronic acid (wt % compared to ACC), and optionally additional one or more stabilizer.

In some embodiments, the combination therapy or combo formulation comprises 0.0075-0.15% (w/v) of hyaluronic acid.

The terms “combination therapy” and “combo formulation” are used herein interchangeably.

In some embodiments, the hyaluronic acid comprises or is high molecular weight hyaluronic acid, for example, having a molecular weight of at least 1,000 kDa, such as 3,000 kDa.

According to some embodiments, there is provided a pharmaceutical composition comprising ACC and hyaluronic acid in a single dosage form, and optionally further comprising at least one stabilizer or a stabilizing agent of ACC, as disclosed herein.

In some embodiments, the pharmaceutical composition is formulated for intraarticular administration.

In some embodiments, the pharmaceutical composition is for use in the reduction of inflammation of the joints in a subject with an inflammatory joint disease. In some embodiments, the pharmaceutical composition if for use in the treatment or prevention of an inflammatory joint disease.

In some embodiments, a reduction comprises at least 5%, at least 15%, at least 25%, at least 35%, at least 50%, at least 65%, at least 75%, at least 85%, or at least 95% reduction, or any value and range therebetween. Each possibility represents a separate embodiment of the invention. In some embodiments, a reduction comprises 5-50%, 10-100%, 20-85%, or 25-90%, reduction. Each possibility represents a separate embodiment of the invention.

In some embodiments, the composition is a nutraceutical composition or a pharmaceutical composition.

In some embodiments, the nutraceutical composition comprises a food supplement or a medical food.

In some embodiments, the ACC stabilized by at least one stabilizing agent and the glatiramer acetate or a pharmaceutically acceptable salt thereof are administered sequentially or simultaneously.

In some embodiments, the ACC stabilized by at least one stabilizing agent and the glatiramer acetate or a pharmaceutically acceptable salt thereof are formulated as a separate dosage form or are co-formulated as a single dosage form.

In some embodiments, there is provided a combined oral administration and/or injection of ACC particles stabilized by at least one stabilizing agent, as disclosed herein, and hyaluronic acid administered by intraarticular injection. In some embodiments, such combined treatment comprises administering the composition of ACC as disclosed herein, daily, and administering hyaluronic acid once a month.

In some embodiments, the method comprises administering a therapeutically effective amount of a pharmaceutical composition comprising ACC particles as disclosed herein, and hyaluronic acid to a subject in need thereof.

In some embodiments, the method comprises administering the pharmaceutical composition comprising ACC and hyaluronic acid via intraarticular administration.

In some embodiments, treatment with ACC is combined with intraarticular injections of hyaluronic acid. In some embodiments, the ACC is administered daily (for example, 1, 2 or 3 times a day), and the hyaluronic acid is administered once a month.

In some embodiments, ACC is administered daily (for example, 1, 2 or 3 times a day), and hyaluronic acid is administered every 4, 5, 6, 7 or 8 weeks. Each possibility represents a separate embodiment of the invention.

In some embodiments, ACC is administered daily (for example, 1, 2 or 3 times a day), and the hyaluronic acid is administered every 1, 2 or 3 months. Each possibility represents a separate embodiment of the invention.

As used herein, the term “subject” according to embodiments of the present invention is a mammal. In some embodiments, the subject is typically a human subject.

### ACC Particles and Compositions

As used herein, the term “particle” as used herein refers to a discrete primary nanoparticle of amorphous calcium carbonate (ACC), as well as to an aggregate or an agglomerate thereof. According to some embodiments, the particle is or comprises a primary particle of ACC. According to some embodiments the primary particle is characterized by having a size ranging from: 5 to 100 nm, 10 to 300 nm, 20 to 500 nm, or 10 to 1,000 nm. Each possibility represents a separate embodiment of the invention.

In some embodiments, the ACC particles are agglomerated particles.

In some embodiments, ACC particle as disclosed herein, comprises an aggregate or an agglomerate of the particles disclosed. In some embodiments, the aggregate or agglomerate of the particles disclosed herein, is at least 3, 10, 100, 1000, or 10,000 times greater in size compared to the particles. In some embodiments, the aggregate or agglomerate of the particles disclosed herein, are referred to as a secondary particle.

In some embodiments, the present invention comprises processing secondary particles, e.g., aggregates or agglomerates, so as to obtain the particles of the invention or a portion thereof. In some embodiments, processing comprises milling. In some embodiments, processing comprises dissolving (such as by various dissolution techniques). In some embodiments, milling provides a smaller aggregate or an agglomerate characterized by having essentially the same order of magnitude size as the particles of the invention. In some embodiments, milling provides a debris of the nanoparticle of the invention (and therefore is smaller in size compared to the particle). In some embodiments, dissolving the aggregate or agglomerate results in the release of a single primary particle of the invention. In some embodiments, the release of particles from the agglomerates/aggregates in the body occurs via the initial dissolution of the bonded area between the particles in the cluster. The bonding area is expected to be more amorphous with lower number of bonds, hence expected to dissolve faster than the primary particles.

In some embodiments, the particles are nanometric particles. In some embodiments, the particles are nanoparticles.

In some embodiments, the nanoparticles are characterized by having a size ranging from 10 to 500 nm, 10 to 550 nm, 10 to 600 nm, 10 to 650 nm, 10 to 700 nm, 10 to 750 nm, 10 to 800 nm, 10 to 850 nm, 10 to 900 nm, 10 to 950 nm, 10 to 975 nm, 10 to 1,000 nm, or 10 to 1,500 nm. Each possibility represents a separate embodiment of the invention.

In some embodiments, a size comprises maximal size. In some embodiments, a size comprises an average size, such as within a composition comprising a plurality of particles. In some embodiments, a size comprises a median size, such as within a composition comprising a plurality of particles.

In some embodiments, nanoparticles is primary particle. In some embodiments, the particle is secondary particle.

In some embodiments, the ACC is substantially soluble in pH equal to or greater than 5.8, equal to or greater than 5.9, equal to or greater than 6.0, equal to or greater than 6.2, equal to or greater than 6.5, equal to or greater than 7.0, equal to or greater than 7.25, equal to or greater than 7.50, equal to or greater than 7.75, or lower than 8.00, or any value and range therebetween. Each possibility represents a separate embodiment of the invention.

In some embodiments, the ACC as disclosed herein is adequately soluble in a pH ranging from 6.00 to below 8.00. In some embodiments, the ACC is essentially non soluble or non-adequately soluble in a pH exceeding 8.00.

As used herein, substantially, refers to at least 5%, at least 15%, or at least 25% of ACC primary particles dissolving from an ACC secondary particle.

In some embodiments, substantially soluble is compared to crystalline calcium carbonate (CCC). In some embodiments, substantially soluble refers to ACC being at least 5%, at least 15%, at least 25%, at least 35%, at least 50%, at least 75%, at least 100%, at least 250%, at least 350%, at least 500%, at least 750%, or at 1,000% more soluble that CCC at a pH equal to or greater than 6.0. or any value and range therebetween. Each possibility represents a separate embodiment of the invention.

In some embodiments, the ACC is coated or encapsulated within an enteric outer layer or a capsule, as disclosed herein.

As used herein, the term “outer layer” refers to an organization wherein a layer of an enteric material cover the ACC particles, granules, pressed tablets. In some embodiments, the enteric material covers a capsule shell. In some embodiments, the enteric material is the capsule shell.

According to some embodiments, there is provided a composition comprising ACC particles stabilized by at least one stabilizing agent, and having size ranging from 10 to 1,000 nm. In some embodiments, the composition is for use in the treatment or prevention of an acidosis-related disease or condition.

In some embodiments, the composition is a solid composition. In some embodiments, the composition is a dry composition. In some embodiments, the composition is in the form of a powder. In some embodiments, the composition is characterized by having a buffering activity. In some embodiments, the composition is a solid buffer composition. In some embodiments, the composition is a dispersion or a suspension. In some embodiments, the composition is an aqueous composition. In some embodiments, the composition is aqueous dispersion or suspension.

In some embodiments, at least 30%, at least 40%, at least 50%, at least 60%, at least 70%, at least 80%, at least 90%, at least 95%, at least 99%, or 100% of the particles of the composition have a size ranging from 10 to 1,000 nm, or any value and range therebetween. Each possibility represents a separate embodiment of the invention. In some embodiments, 10-100%, 20-99%, 30-80%, 40-90%, 50-75%, or 60-85% of the particles of the composition have a size ranging from 10 to 1,000 nm. Each possibility represents a separate embodiment of the invention.

In some embodiments, at least 30%, at least 40%, at least 50%, at least 60%, at least 70%, at least 80%, at least 90%, at least 95%, at least 99%, or 100% of the particles of the composition have a size ranging from 10 to 100 nm, or any value and range therebetween. Each possibility represents a separate embodiment of the invention. In some embodiments, 10-100%, 20-99%, 30-80%, 40-90%, 50-75%, or 60-85% of the particles of the composition have a size ranging from 10 to 1,000 nm. Each possibility represents a separate embodiment of the invention.

In some embodiments, at least 30% of the ACC particles stabilized by at least one stabilizing agent comprise primary particles having a maximal size ranging from 10 to 500 nm.

In some embodiments, the composition further comprises an excipient, a carrier, or a diluent. In some embodiments, the composition further comprises an anti-caking agent, an excipient, a disintegrant, a biner, a flavoring agent, a lubricant, or any combination thereof.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the term “anti-caking agent” refers to any compound that when added to a powdered or granulated material, inhibits, reduces, or prevents, the formation of aggregates, agglomerates, or lumps.

In some embodiments, the composition is a nutraceutical composition or a pharmaceutical composition.

In some embodiments, the nutraceutical composition comprises a food supplement or a medical food.

According to some embodiments, ACC particles stabilized by at least one stabilizing agent are an active agent of the herein disclosed pharmaceutical composition. According to some embodiments, ACC particles stabilized by at least one stabilizing agent are the sole active agent of the herein disclosed pharmaceutical composition.

In some embodiments, the pharmaceutical composition is an oral composition.

As used herein, the term “pharmaceutical composition” refers to any composition comprising a stabilized ACC and a pharmaceutically acceptable excipient.

As used herein, the terms “pharmaceutically acceptable carrier” or “pharmaceutically acceptable excipient” refer to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions.

The terms “pharmaceutically acceptable” and “pharmacologically acceptable” include molecular entities and compositions that do not in general produce an adverse, allergic, or other untoward reactions when administered to an animal, or human, as appropriate. For human administration, preparations should meet sterility, pyrogenicity, general safety, and purity standards as required by a government drug regulatory agency, e.g., the United States Food and Drug Administration (FDA) Office of Biologics standards.

In some embodiments, the pharmaceutical composition comprising ACC particles stabilized by at least one stabilizing agent, as disclosed herein, is formulated for transmucosal administration. In some embodiments, the pharmaceutical composition comprising ACC particles stabilized by at least one stabilizing agent, as disclosed herein, is formulated for specific delivery and/or absorption in mildly acidic to neutral pH environment. In some embodiments, the pharmaceutical composition comprising ACC particles stabilized by at least one stabilizing agent, as disclosed herein, is formulated for specific delivery and/or absorption in a site lined or covered with epithelial tissue and/or epithelial cells. In some embodiments, the epithelial tissue comprises simple epithelium or epithelial cells. In some embodiments, simple epithelium comprises simple squamous epithelium. In some embodiments, the epithelial tissue comprises skin. In some embodiments, the epithelial tissue is a respiratory epithelium, e.g., pseudo-columnar and/or ciliated epithelium. In some embodiments, the epithelial tissue is an absorbing epithelium, e.g., simple columnar epithelium. In some embodiments, the pharmaceutical composition comprising ACC particles stabilized by at least one stabilizing agent, as disclosed herein, is absorbed primarily or predominantly in a site lined or covered with epithelial tissue and/or epithelial cells. In some embodiments, the pharmaceutical composition comprising ACC particles stabilized by at least one stabilizing agent, as disclosed herein, is formulated for specific delivery and/or absorption in a site lined or covered with endothelial cells. In some embodiments, the endothelial cells are endothelial cells of a blood vessel, such as, but not limited to a vein.

As used herein, the term “nutraceutical composition” refers to a composition suitable for use in a subject, such as, but not limited to, a human subject, or an animal, comprising one or more natural products with therapeutic action which provide a health benefit or have been associated with disease prevention or reduction.

The term “food supplement” is used to mean a product containing the composition and intended to supplement the food by providing nutrients that are beneficial to health according to any acceptable directive, such as European directive. For example, a food supplement may be a capsule or a tablet for swallowing, or a powder or small vial to mix with a food and providing beneficial health effects.

As used herein, the term “medical food” refers to a food item specially formulated for the dietary management of a disease or disorder in a subject.

According to some embodiments, the composition is a delayed release composition thereby the use comprises delayed release administration of ACC particles stabilized by at least one stabilizing agent.

As used herein, the terms “controlled release” and “modified release” are interchangeable and refer to a composition or dosage form which comprises an active drug and which is formulated to provide a release of the active ingredient according to a desired profile, which is different from immediate release. The above-mentioned terms comprise composition providing sustained-release, extended-release, prolonged-release, delayed release, and any combinations of modified release profiles such as extended and delayed release, of the active ingredient.

According to some embodiments, the composition is formulated as a delayed release composition. The term “delayed release composition” refers to a composition formulated to release discrete portion or portions of drug at a time other than promptly after administration, for examples after passing a particular part of the gastrointestinal tract, e.g., after passing the stomach. According to one embodiment, the composition of the present invention releases stable ACC after passing stomach. According to one embodiment, the composition of the present invention releases ACC particles stabilized by at least one stabilizing agent only after passing stomach. According to one embodiment, the composition of the present invention releases ACC particles stabilized by at least one stabilizing agent in the gastrointestinal tract. According to one embodiment, the composition of the present invention releases ACC particles stabilized by at least one stabilizing agent only in the gastrointestinal tract and only after passing stomach.

According to some embodiments, the delayed release of the ACC particles stabilized by at least one stabilizing agent involves coating (or otherwise encapsulating) with a substance, which is not absorbed, or otherwise broken down, by the gastric fluids to release said active ingredient until a specific desired point in the intestinal tract is reached. According to some embodiments, the delayed-release formulation for use herein is achieved by coating a tablet, capsule, particles, granules, pellets, or beads of active ingredient with a coating, or by placing the active ingredient in a capsule shell comprising a substance which is pH-dependent, e.g., broken down at a pH which is generally present in the small intestine, but not broken down at a pH which is generally present in stomach.

The term “enteric coating” comprises any pharmaceutically acceptable coating preventing the release of the active agent in the stomach or acidically comparable environment, and sufficiently disintegrating in the intestine tract (by contact with approximately neutral or alkaline intestine juices) to allow the resorption of the active agent through the walls of the intestinal tract. Various in vitro tests for determining whether or not a coating is classified as an enteric coating have been published in the pharmacopoeia of various countries. More specifically, the term “enteric coating” as used herein refers to a coating which remains intact for at least 2 hours, in contact with artificial gastric juices such as HCl of pH 1 at 36 to 38° C. and preferably thereafter disintegrates within 30 minutes in artificial intestinal juices such as a KH2 PO4 buffered solution of pH 6.8.

In some embodiments, the enteric coat comprises or is selected from: Methyl acrylate-methacrylic acid copolymer, Cellulose acetate phthalate (CAP), Cellulose acetate succinate, Hydroxypropyl methyl cellulose phthalate, Hydroxypropyl methyl cellulose acetate succinate (hypromellose acetate succinate), Polyvinyl acetate phthalate (PVAP), Methyl methacrylate-methacrylic acid copolymers, Shellac, Cellulose acetate trimellitate, Sodium alginate, Zein, enteric coating aqueous solution (such as ethylcellulose, medium chain triglycerides [coconut], oleic acid, sodium alginate, stearic acid), and coated softgels.

In some embodiments, the enteric coat comprises cellulose. In some embodiments, the enteric coating comprises a cellulosic enteric derivative. Enteric capsules used in the examples are Vcaps® Enteric Capsugel.

The term “enteric coated capsule” as used herein refers to capsules surrounding the internal content, wherein the capsule has been treated or is prepared from a polymer resistant to decomposition in the acid conditions of the stomach.

According to one embodiment, the composition of the present invention comprises solid ACC particles stabilized by at least one stabilizing agent, such as pellets, powder, or granules of any other type of ACC particles stabilized by at least one stabilizing agent, coated individually, or as a secondary particle, or as an agglomerate or aggregate of secondary particles, with enteric coating. According to some embodiments, such coated particles may be then pressed into tablet. According to one embodiment, such tablets are then coated with enteric coating. Alternatively, such coated particle may be then filling within enteric coating capsule or used as is.

According to another embodiments, the solid composition comprising ACC particles stabilized by at least one stabilizing agent is present in the form of a tablet coated with an enteric coating.

According to another embodiment, the solid composition comprising ACC particles stabilized by at least one stabilizing agent is present in the form of particles such as powder filled within enteric coating capsule.

As used herein, the term “pellet” refers to any particle that is prepared by process of agglomeration of, for example, powder.

According to any one of the above embodiments, ACC particles stabilized by at least one stabilizing agent are a sole active agent. According to some embodiments, the present invention provides an oral composition comprising ACC particles stabilized by at least one stabilizing agent as a single active agent, for use in the treatment or prevention of an acidosis-related disease or condition, as described herein. According to some embodiments, the composition consists essentially of ACC particles stabilized by at least one stabilizing agent. In some embodiments, the composition consisting essentially of ACC particles stabilized by at least one stabilizing agent as an active agent, further comprises non-active excipients.

As used herein, the term “consisting essentially of” refers to that a given compound or substance constitutes the vast majority of the active ingredient's portion or fraction of the composition.

In some embodiments, consisting essentially of means that the ACC particles stabilized by at least one stabilizing agent of the invention constitute at least 80%, at least 90%, at least 95%, at least 98%, at least 99%, or at least 99.9% by weight, of the active ingredient(s) of the composition, or any value and range therebetween. Each possibility represents a separate embodiment of the invention.

The terms “amorphous calcium carbonate”, “ACC”, “stable ACC”, “stabilized ACC”, and “ACC particles stabilized by at least one stabilizing agent” are used herein interchangeably and refer to the amorphous polymorph of calcium carbonate.

As used herein, the terms “stable” and “stabilized” indicate that the calcium carbonate is maintained in the amorphous form for a long period of time, for example for about at least 7 days in the solid form having less than or about 30% crystalline calcium carbonate. According to some embodiments, the composition is stable for at least 7 days, at least 1 month, at least 3 months, at least 6 months, at least 1 year, or at least 2 years, including any value and range therebetween. Each possibility represents a separate embodiment of the invention.

In some embodiments, the composition is stable for 7 days to 2 months, 1 months to 6 months, 4 months to 18 months, 6 months to 24 months, or 7 days to 18 months. Each possibility represents a separate embodiment of the invention.

Methods for determining stability of ACC are common and would be apparent to one of skill in the art. Such methods are exemplified herein.

As used herein, the term “stable” refers to calcium carbonate that is maintained as ACC for a period of time of at least 12 hr, at least 24 hr, at least 48 hr, at least 7 days, or at least 30 days, or any value and range therebetween, in a water suspension, and characterized by having less than 30% by weight, less than 25% by weight, less than 20% by weight, less than 15% by weight, less than 10% by weight, less than 5% by weight, less than 3% by weight, less than 2% by weight, or less than 1% by weight crystalline calcium carbonate, or any value and range therebetween, when placed in a moisture resistant container at room temperature. Each possibility represents a separate embodiment of the invention.

In some embodiments, a composition being a suspension comprises the ACC nanoparticles as described herein, is stable for 2 days to 14 days, 2 days to 28 days, 2 days to 1 month, 5 days to 35 days, 1 week to 8 weeks, 2 days to 2 months, and 3 days to 3 month, wherein stable is at room temperature or ambient temperature. Each possibility represents a separate embodiment of the invention. In some embodiments, a composition, being a suspension comprises the ACC nanoparticles as described herein, is stable for 2 days to 14 days, 2 days to 28 days, 2 days to 1 month, 5 days to 35 days, 1 week to 8 weeks, 2 days to 2 months, and 3 days to 3 month, wherein stable comprises at least 50%, at least 60%, at least 70%, at least 80%, at least 90%, at least 95%, at least 97%, at least 99%, or 100%, of the nanoparticles in the suspension are in an amorphous form or phase, or any value and range therebetween. Each possibility represents a separate embodiment of the invention.

According to some embodiments, ACC stabilized by at least one stabilizer is a natural ACC.

The term “natural ACC” as used herein refers to any ACC isolated or derived from a natural source. Non-limiting examples of natural sources of ACC include gastroliths of freshwater crustaceans. In certain embodiments, the naturally occurring ACC source includes gastrolith organs, or a portion thereof ground to a fine powder, essentially as described in WO 2005/115414. Optionally, ACC comprises a combination of naturally occurring and synthesized ACC.

According to some embodiments, ACC is a synthetic ACC.

The term “synthetic ACC” as used herein generally refers to any ACC produced by man ex-vivo or in vitro.

According to some embodiments, the ACC is a synthetic ACC stabilized by at least one stabilizer as defined herein below.

In some embodiments, ACC is a chemically synthesized ACC. In some embodiments, ACC is a biosynthetic ACC.

As used herein, the term “plurality” encompasses any integer equal to or greater than 2.

According to some embodiments, the composition of the invention, and a method for preparing same, is described in Tables 19, 21, and 22, disclosed herein.

In some embodiments, the at least one stabilizing agent is selected from: inorganic polyphosphates, inorganic phosphates organic acids, organic acids, phosphorylated, phosphonated, sulfated or sulfonated organic compound, phosphoric or sulfuric esters of hydroxy carboxylic acids, phosphorylated amino acids, bisphosphonate, organic polyphosphate, hydroxyl bearing organic compounds, derivatives thereof, proteins, and any combinations thereof. According to some embodiments, the stabilizer is selected from: phosphoserine, adenosine triphosphate, adenosine diphosphate, phytic acid, citric acid, etidronic acid, pyrophosphate, polyphosphate, triphosphate, hexamethaphosphate, ethanol, and any combination thereof. According to some embodiments, the present invention provides a pharmaceutical composition comprising a drug suitable for the treatment or prevention of an acidosis-related disease or condition and ACC stabilized by triphosphate, such as sodium triphosphate, and citric acid.

As used herein, the term “organic polyphosphate” refers to an organic compound that contains 2 or more phosphate groups bonded to the organic compound through C—O—P bond, as an example—phytic acid.

As used herein, the term “polyphosphonate” refers to an organic compound containing 2 or more phosphonate groups bonded by C—P bonds to the organic compound.

### Combination Therapy

According to some embodiments, there is provided a combination therapy comprising ACC particles stabilized by at least one stabilizing agent, and at least one additional active agent or a drug. In some embodiments, the additional drug is suitable for the treatment or prevention of an acidosis-related disease.

According to some embodiments, there is provided a combination therapy comprising ACC particles stabilized by at least one stabilizing agent and multiple sclerosis (MS) treating compound.

MS treating compounds would be apparent to one of ordinary skill in the art and include glatiramer acetate or any pharmaceutically acceptable salt thereof.

In some embodiments, the MS treating compound is or comprises glatiramer acetate or any pharmaceutically acceptable salt thereof.

According to some embodiments, there is provided a combination comprising: (a) ACC stabilized by at least one stabilizing agent; and (b) glatiramer acetate or a pharmaceutically acceptable salt thereof, for use in the treatment of multiple sclerosis in a subject in need thereof.

As used herein, the terms “multiple sclerosis” and “MS” refers to an inflammatory autoimmune disease of the central nerve system (CNS) wherein the nerve insulating myelin sheath is partially lost, thereby resulting in various pathological symptoms. MS includes various types of the disease such as relapsing/remitting (RRMS), secondary progressive (SPMS), progressive relapsing (PRMS) and primary progressive (PPMS). The first symptoms which appear at the onset of MS are referred to herein at times as “MS-related symptoms”. The symptoms of MS in EAE-induced animals (animal model of MS) are typically weakness and malfunction in the animal's tail, followed by weakness of its rear feet and finally weakness in its front feet. In humans, such first MS-related symptoms may typically be double vision, facial numbness, facial weakness, vertigo, nausea, vomiting ataxia, weakness of the arms, etc.

According to some embodiments, the combination may be administered by any known method. The term “administering” or “administration of” a substance, a compound or a composition to a subject can be carried out using one of a variety of methods known to those skilled in the art. For example, a compound or a composition can be administered enterally or parenterally. Enterally refers to administration via the gastrointestinal tract including per os, sublingually or rectally. Parenteral administration includes administration intravenously, intradermally, intramuscularly, intraperitoneally, topical, subcutaneously, ocularly, sublingually, intranasally, by inhalation, intraspinally, intracerebrally, and transdermally (by absorption, e.g., through a skin duct). A compound or a composition can also appropriately be introduced by rechargeable or biodegradable polymeric devices or other devices, e.g., patches and pumps, or formulations, which provide for the extended, slow, or controlled release of the compound or composition. Administering can also be performed, for example, once, a plurality of times, and/or over one or more extended periods. In some embodiments, the administration includes both direct administration, including self-administration, and indirect administration, including the act of prescribing a drug or a medical food. For example, as used herein, a physician who instructs a patient to self-administer a drug or a medical food, or to have the drug or the medical food administered by another and/or who provides a patient with a prescription for a drug or a medical food is administering the drug or a medical food to the patient.

According to some embodiments, the treating comprises inhibiting the progression of MS. According to another embodiment, treating comprises ameliorating the symptoms of the disease. According to some embodiments, the subject suffers from relapsing/remitting MS.

The present invention provides in another aspect, a method for treating multiple sclerosis in a subject in need thereof comprising administering to said subject a composition comprising amorphous calcium carbonate (ACC) stabilized by at least one stabilizer. According to one embodiment, the method further comprises co-administration of the stabilized ACC and an effective amount of glatiramer acetate. Thus, according to one embodiment, the present invention provides a method of treating multiple sclerosis in a subject in need thereof comprising co-administering to said subject a composition comprising amorphous calcium carbonate (ACC) stabilized by at least one stabilizer, and an effective amount of a glatiramer acetate. According to one embodiment, the method comprises co-administration of the stabilized ACC and an effective amount of a glatiramer acetate. Thus, according to some embodiments, the present invention provides a method for treating multiple sclerosis in a subject in need thereof comprising co-administering to said subject an effective amount of a glatiramer acetate and a composition comprising amorphous calcium carbonate (ACC) stabilized by at least one stabilizer.

In some embodiments, the ACC stabilized by at least one stabilizing agent and the glatiramer acetate or a pharmaceutically acceptable salt thereof are administered sequentially or simultaneously.

In some embodiments, the ACC stabilized by at least one stabilizing agent and the glatiramer acetate or a pharmaceutically acceptable salt thereof are formulated as a separate dosage form or are co-formulated as a single dosage form. In some embodiments, the ACC stabilized by at least one stabilizing agent and MS treating compound are formulated individually, wherein the stable ACC is in a first pharmaceutical composition, and wherein the MS treating compound is in a second pharmaceutical composition. In some embodiments, the stable ACC and MS treating compound are provided concomitantly or separately.

The term “glatiramer acetate” as used herein refers to a compound known as Copolymer 1 that is sold under the trade name Copaxone® and consists of the acetate salts of synthetic polypeptides, containing four naturally occurring amino acids: L-glutamic acid, L-alanine, L-tyrosine, and L-lysine. In some embodiments, the amino acids have an average molar fraction of 0.141, 0.427, 0.095, and 0.338, respectively. The average molecular weight of glatiramer acetate in Copaxone® is 4,700-11,000 Daltons (FDA Copaxone® label) and the number of amino acid ranges between about 15 to about 100 amino acids. The term also refers to chemical derivatives and analogues of the compound. Typically, the compound is prepared and characterized as specified in any of U.S. Pat. Nos. 5,981,589; 6,054,430; 6,342,476; 6,362,161; 6,620,847; and 6,939,539, the contents of each of these references are hereby incorporated in their entirety.

In some embodiments, the composition may comprise any other pharmaceutically acceptable salt of glatiramer including, but not limited to, sulfate, pyrosulfate, bisulfate, sulfite, bisulfite, phosphate, monohydrogenphosphate, dihydrogenphosphate, metaphosphate, pyrophosphate, hydrochloride, hydrobromide, hydroiodide, acetate, nitrate, propionate, decanoate, caprylate, acrylate, formate, isobutyrate, caprate, heptanoate, propiolate, oxalate, malonate, succinate, tocopheryl succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-2-sulfonate, p-toluenesulfonate, mandelate and the like salts. Each possibility represents a separate embodiment of the invention.

The term “co-administration” as used herein has the meaning of (i) administering two or more compound in a regimen selected from a single combined administration, (ii) separate individual compositions administered substantially at the same time, and (iii) separate individual compositions administered under separate schedules and include treatment regimens in which the compounds are not necessarily administered by the same route of administration or at the same time. In some embodiments, the agents can be administered in a sequential manner in either order.

The term “sequential administration” refers to an administration of two compounds at different times, and optionally in different modes of administration. In some embodiments, the agents are administered in a sequential manner in either order.

The terms “simultaneous administration” refers to administration of two compounds with only a short time interval between them. In some embodiments, the time interval is in the range of from 0.01 to 60 minutes. According to another embodiment, the combination is administered in a simultaneous manner, e.g., the compounds are administered at the same time.

According to some embodiments, the dosage form of the present invention such as a pharmaceutical composition is suitable for administration via a route selected from: subcutaneous, intravenous, oral, rectal, intramuscular, intraperitoneal, intranasal, intraarterial, intravesical, intraocular, transdermal, topical, or any combination thereof. According to one embodiment, the composition is administered orally. According to another embodiment, the composition is administered parenterally.

According to some embodiment, the ACC stabilized by at least one stabilizing agent and glatiramer acetate are administered in different routes of administration. According to one embodiment, glatiramer acetate is administered subcutaneously, and the ACC stabilized by at least one stabilizing agent is administered orally.

According to one embodiment, the ACC stabilized by at least one stabilizing agent is formulated for oral administration and glatiramer acetate is formulated for parenteral administration, e.g., for subcutaneous administration.

According to some embodiments, the glatiramer acetate and the stable ACC are formulated as a single dosage form in a pharmaceutical composition.

According to some embodiments, the co-administration of glatiramer acetate and the ACC stabilized by at least one stabilizing agent comprises administering glatiramer acetate in a lower dose than the effective dose of glatiramer acetate required when administered alone while maintaining the same therapeutic effect. The typical dose of glatiramer acetate is 20 mg/day or 40 mg 3 times a week (every 48 hours). According to some embodiments, the dose of glatiramer acetate when administered in the herein disclosed combination is at least 10%, at least 20%, at least 30%, at least 40%, at least 50%, at least 60%, or at least 80% lower than the standard effective dose of glatiramer acetate, or any value and range therebetween. Each possibility represents a separate embodiment of the invention.

According to some embodiments, the dose of glatiramer acetate when administered in combination as disclosed herein is 10-50%, 20-70%, or 15-80% lower than the standard effective dose of glatiramer acetate. Each possibility represents a separate embodiment of the invention.

According to one embodiment, the dose of glatiramer acetate when administered in combination with ACC stabilized by at least one stabilizing agent is at least 1.5, 2, 2.5 or 3 times lower than the standard effective dose of glatiramer acetate, or any value and range therebetween. Each possibility represents a separate embodiment of the invention. According to some embodiments, the glatiramer acetate in combination with ACC stabilized by at least one stabilizing agent is administered in a dose of 5-40 mg 2 times a week, 20-100 mg 3 times a week, or 1-40 mg every 96 or 120 hours. Each possibility represents a separate embodiment of the invention.

According to other embodiments, the co-administration of ACC stabilized by at least one stabilizing agent and an additional drug suitable for the treatment or prevention of an acidosis-related disease provides reduced side-effects derived from or attributed to the additional drug.

According to some embodiments, the co-administration of glatiramer acetate or any pharmaceutically acceptable salt thereof and ACC stabilized by at least one stabilizing agent reduces any side-effect derived from or attributed to glatiramer acetate or any pharmaceutically acceptable salt thereof.

### Amorphous Calcium Carbonate Stabilizers

The stabilizer may comprise a molecule having one or more functional groups selected from, but not limited to, hydroxyl, carboxylic, amine, phosphino, phosphono, phosphonato, phosphate, sulfate or sulfino groups, and salts thereof. The hydroxy bearing compounds, combined with metal hydroxide, optionally also bear other functions like carboxylic, etc., but with the hydroxyl not being esterified.

According to some embodiments, the stabilizer has low toxicity or no toxicity to mammalian cells or organism, and in particular to a human being. According to some embodiments, the stabilizer is of food, nutraceutical, or pharmaceutical grade.

In certain embodiments, the ACC stabilizing agent is independently at each occurrence, an organic acid, phosphorylated, phosphonated, sulfated or sulfonated organic compound, phosphoric or sulfuric ester of a hydroxyl carboxylic acid, an organoamine compound, an organic compound comprising a hydroxyl, an organophosphorous compound or salts thereof, phosphorylated amino acids and derivatives thereof, a bisphosphonate compound, an organophosphate compound e.g., phytic acid, an organophosphonate compound, an organophosphorous acids, and salts thereof, an organic compound having multiple functional groups as defined above, an inorganic phosphate and polyphosphate compound, an organic compound having a polyphosphate chain, an organic surfactant, a bio-essential inorganic ion, or any combination thereof.

According to some embodiments, the stabilizer is an organic acid. According to some embodiments, the organic acid is selected from: ascorbic acid, citric acid, lactic acid, acetic acid, oxalic acid, malonic acid, glutaconic acid, succinic acid, maleic acid, aconitic acid, tartaric, glutaric, malic, pyruvic, oxaloacetate, other natural carboxylic or carboxylate compounds, and optionally include compounds having at least two carboxylic groups and molecular weight not larger than 250 g/mol, such as citric acid, tartaric acid, malic acid, etc. According to one particular embodiment, the stabilizer is citric acid.

In another embodiment, the phosphoric ester of hydroxyl carboxylic acids is a phosphoenolpyruvate. In another embodiment, the phosphoric or sulfuric esters of hydroxyl carboxylic acids comprise amino acids, e.g., phosphorylated amino acids. Examples of such esters are phosphoserine, phosphothreonine, sulfoserine, sulfothreonine and phosphocreatine.

The hydroxyl bearing compounds combined with hydroxide may comprise, for example, mono-, di- tri-, oligo-, and polysaccharides like sucrose or other polyols like glycerol. The hydroxyl bearing compounds may further comprise hydroxy acids like citric acid, tartaric acid, malic acid, etc., or hydroxyl-bearing amino acids such as serine or threonine. Each possibility represents a separate embodiment, of the present invention.

Some specific unlimited examples for such ACC stabilizers include phytic acid, citric acid, sodium pyrophosphate dibasic, adenosine 5′-monophosphate (AMP) sodium salt, adenosine 5′-diphosphate (ADP) sodium salt and adenosine 5′-triphosphate (ATP) disodium salt hydrate, phosphoserine, phosphorylated amino acids, food grade surfactants, sodium stearoyl lactylate, and combinations thereof.

According to some embodiments, the stabilizer comprises at least one component selected from phosphoric or sulfuric esters of hydroxyl carboxylic acids, such as phosphoenolpyruvate, phosphoserine, phosphorthreonine, sulfoserine or sulfothreonine and hydroxyl bearing organic compounds, selected from mono-, di-, tri-, oligo- and polysaccharides, for example, sucrose, mannose, glucose. The hydroxyl bearing compound may further comprise at least one alkali hydroxide, such as sodium hydroxide, potassium hydroxide and the like. The phosphorylated acids may be present in oligopeptides and polypeptides. In other embodiments, of the invention, the stabilizer is an organic acid selected from monocarboxylic acid or multiple carboxylic acid, e.g., dicarboxylic acid or tricarboxylic acid. Each possibility represents a separate embodiment, of the invention. The organic acid may be as defined herein.

In some embodiments, the ACC stabilizer is selected from phosphorylated amino acids, polyols, and combinations thereof. In some embodiments, the stable ACC comprises a phosphorylated compound as a stabilizer wherein the phosphorylation is performed on the hydroxyl group of an organic compound. In some embodiments, the stable ACC comprises a stabilizer selected from: citric acid, phosphoserine, phosphothreonine and combinations thereof. The non-limiting examples of stabilizers containing phosphate, phosphite, phosphonate groups and salts or esters thereof include phytic acid, dimethyl phosphate, monomethyl phosphate, sodium pyrophosphate, multiethyl pyrophosphate, ribulose bisphosphate, etidronic acid and other medical bisphosphonates, 3-phosphoglyceric acid salt, glyceraldehyde 3-phosphate, 1-deoxy-D-xylulose-5-phosphate sodium salt, diethylene triamine pentakis(methylphosphonic acid), nitrilotri(methylphosphonic acid), 5-phospho-D-ribose 1-diphosphate pentasodium salt, adenosine 5′-diphosphate sodium salt, adenosine 5′-triphosphate disodium salt hydrate, α-D-galactosamine 1-phosphate, 2-phospho-L-ascorbic acid trisodium salt, α-D-galactose 1-phosphate dipotassium salt pentahydrate, α-D-galactosamine 1-phosphate, O-phosphorylethanolamine, disodium salt hydrate, 2,3-diphospho-D-glyceric acid pentasodium salt, phospho(enol)pyruvic acid monosodium salt hydrate, D-glyceraldehyde 3-phosphate, sn-glycerol 3-phosphate lithium salt, D-(−)-3-phosphoglyceric acid disodium salt, D-glucose 6-phosphate sodium salt, phosphatidic acid, ibandronate sodium salt, phosphonoacetic acid, DL-2-amino-3-phosphonopropionic acid or combinations thereof. The bio-essential inorganic ions may include, inter alia, Na, K, P, S, N, P or S in the phase of oxides, or N as ammonia or nitro groups.

According to some embodiments, the stabilizer is a polyphosphate or pharmaceutically acceptable salts thereof. According to some embodiments, the polyphosphate is physiologically compatible, water soluble polyphosphate salt selected from the group consisting of sodium, potassium, and any other essential cation of polyphosphate. In one embodiment, the polyphosphate is organic or inorganic polyphosphate. The term “polyphosphate” as used herein refers to polymeric esters of PO₄. According to some embodiments, the polyphosphate is physiologically compatible water-soluble polyphosphate salt selected from the group consisting of sodium and potassium polyphosphate. In some embodiments, the polyphosphate is an inorganic polyphosphate or pharmaceutically acceptable salts thereof. Not-limiting examples of such salt are Na, and K According to some embodiments, the inorganic polyphosphate comprises 2 to 10 phosphate groups, e.g., 2, 3, 4, 5, 6, 7, 8, 9, or 10 phosphate group. According to some embodiments, the polyphosphate is selected from pyrophosphate, triphosphate, and hexametaphosphate. According to one embodiment, the stabilizer is pyrophosphate or pharmaceutically acceptable salts thereof such as sodium pyrophosphate. According to another embodiment, the stabilizer is triphosphate or pharmaceutically acceptable salts thereof such as sodium triphosphate. The terms “triphosphate”, “polytriphosphate”, and “tripolyphosphate” are used herein interchangeably. According to a further embodiment, the stabilizer is hexametaphosphate or a pharmaceutically acceptable salt thereof such sodium hexametaphosphate.

According to some embodiments, the stabilizer is a bisphosphonate or pharmaceutically acceptable salts thereof. The not-limiting examples of salt are Na, K, and Mg.

The term “bisphosphonate” as used herein refers to organic compounds having two phosphonate [PO(OH)₂] groups. The term further relates to compounds having a backbone of PO₃-organic-PO₃. Most typical is a series of bisphosphonates that are used as pharmaceuticals for treating osteoporosis. According to some embodiments, the bisphosphonate is selected from the group consisting of etidronic acid, zoledronic acid, medronic acid, alendronic acid and a pharmaceutically acceptable salt thereof. According to some embodiments, the stabilizer is an etidronic acid or a pharmaceutically acceptable salt thereof. According to another embodiment, the stabilizer is a zoledronic acid or a pharmaceutically acceptable salt thereof. According to a further embodiment, the stabilizer is a medronic acid or a pharmaceutically acceptable salt thereof. According to certain embodiments, the stabilizer is alendronic acid or a pharmaceutically acceptable salt thereof.

According to certain embodiments, the stabilizer is a phosphorylated amino acid. According to one embodiment, the phosphorylated amino acid is phosphoserine. According to another embodiment, the phosphorylated amino acid is phosphothreonine.

According to some embodiments, the stabilizer is polyphosphate or a bisphosphonate as defined hereinabove, and the molar ratio between P atoms of the stabilizer and Ca atoms of the ACC (P:Ca molar ratio) is about 1:90 to 1:1. In one embodiment, the P:Ca molar ratio is about 1:40 to about 1:1. In some embodiments, the P:Ca molar ratio is about 1:35 to about 1:2. In some embodiments, the P:Ca molar ratio is about 1:30 to about 1:3. In some embodiments, the P:Ca molar ratio is about 1:28 to about 1:3. In some embodiments, the P:Ca molar ratio is about 1:25 to about 1:4. In some embodiments, the P:Ca molar ratio is about 1:20 to about 1:5. In some embodiments, the P:Ca molar ratio is about 1:20 to about 1:6. In some embodiments, the P:Ca molar ratio is about 1:15 to about 1:5. In some embodiments, the P:Ca molar ratio is about 1:25 to about 1:5. According to some embodiments, such polyphosphate is pyrophosphate, triphosphate, hexametaphosphate or a pharmaceutically acceptable salt thereof. According to another embodiment, the bisphosphonate is alendronic acid, etidronic acid, zoledronic acid or medronic acid and the P:Ca molar ratio is as defined herein.

According to some embodiments, the calcium content (Ca content) of such composition comprising polyphosphate or bisphosphonate as a stabilizer is about 1 wt % to about 39 wt %, about 5 wt % to about 39 wt %, about 10% to about 39 wt %, about 15% to about 39 wt %, about 20 wt % to about 38 wt %, about 25 wt % to about 38 wt %, or about 30 to about 38. The terms “Ca content” and “calcium content” is used herein interchangeably and refer to the content of calcium of the ACC in the final composition.

In certain embodiments, the P:Ca molar ratio is about 1:40 to about 1:1, and the Ca content is about 20 wt % to about 39 wt %. In some embodiments, the molar ratio is 1:28 to about 1:3, and the Ca content is about 30 wt % to about 38 wt %. In another embodiment, the molar ratio is 1:25 to about 1:5, and the Ca content is about 30 wt % to about 36 wt %.

According to some embodiments, the stabilizer is selected from: a polyphosphate, phosphorylated amino acid, bisphosphonate, citric acid, tartaric acid, and any combination thereof. According to one embodiment, the polyphosphate is selected from: triphosphate, pyrophosphate, and hexametaphosphate, the phosphorylated amino acid is phosphoserine or phosphothreonine, and the bisphosphonate is selected from: alendronate, etidronic acid, zoledronic acid and medronic acid.

According to some embodiments, the stabilizer is polyphosphate or bisphosphonate and the molar ratio between P atoms of the stabilizer and Ca atoms of the ACC is about 1:90 to 1:1.

The stabilized ACC may be stabilized by more than one stabilizer, e.g., two or more stabilizers. In some embodiments, more than one stabilizer, e.g., 2, 3 or 4 stabilizers are added. In some embodiments, the first stabilizer and the second stabilizer are similar. In other embodiments, the first stabilizer and the second stabilizer are different stabilizers. The first and the second stabilizers may be each independently as defined herein. The stable ACC can comprise more than two stabilizers, wherein one or more stabilizers are added to the ACC during the formation and precipitation of the ACC.

According to one embodiment, ACC is stabilized by a combination of phosphoserine and citric acid. According to another embodiment, ACC is stabilized by a combination of triphosphate and citric acid.

### General

The terms “comprising”, “comprise(s)”, “include(s)”, “having”, “has”, and “contain(s)” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step, or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of ±10%, or ±5%, ±1%, or even ±0.1% from the specified value.

The term “or” as used herein, denotes alternatives that may, where appropriate, be combined; that is, the term “or” includes each listed alternative separately as well as their combination if the combination is not mutually exclusive.

Having now generally described the invention, the same will be more readily understood through reference to the following examples, which are provided by way of illustration and are not intended to be limiting of the present invention.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Effects of Various ACC and CCC Suspensions on Acidified Serum in Medium

This experiment evaluates the ability of ACC, formulated with different stabilizers, to affect the pH of medium supplemented with 10% (v/v) serum. The example demonstrates the rapid pH response when the serum is acidified at levels found around tumors and inflammations. It also demonstrates that the level of the final pH control is feasible by a stabilized ACC, hence serving as a solid buffer, at levels suitable for body environments.

To 18 ml of medium DMEM/F12 (Biological Industries, Beit Haemek, Israel), 2 ml of fetal bovine serum (FBS, Biological Industries, Beit Haemek, Israel) was added. The solution was placed inside a sterile tissue-sample cup, and a hole was made at the top of the cup to which a pH probe was inserted. The cup was placed on a magnetic stirrer (JB-10 stirrer, Inesa, China) and the solution was constantly stirred during the measurement.

A pH meter (MesuLab, PXSJ-216F ion meter, MRC, Israel) was connected to a PC the data logging of the pH measurements was performed using the software REXDC2.0.

After setting the system and starting the pH measurement and data logging, an amount of 20.5 μl of lactic acid was added to the solution in order to reduce the pH to a slightly acidic pH. After a few seconds and the stabilization of the pH, an amount of 3 ml of freshly prepared ACC suspension was added to the solution. This experiment was repeated several times. In the first experiment ACC stabilized with triphosphate (TP), either in an in-situ freshly prepared suspension or as a solid powder, redispersed in water, were compared to crystalline calcium carbonate (CCC), dispersed in water. In another experiment, two ACC suspensions containing 1% elemental calcium were stabilized with citric acid (CA; 20% or 15% Ca) were evaluated. The content of calcium measured by titration for these ACC suspensions after filtration was 0.3%.

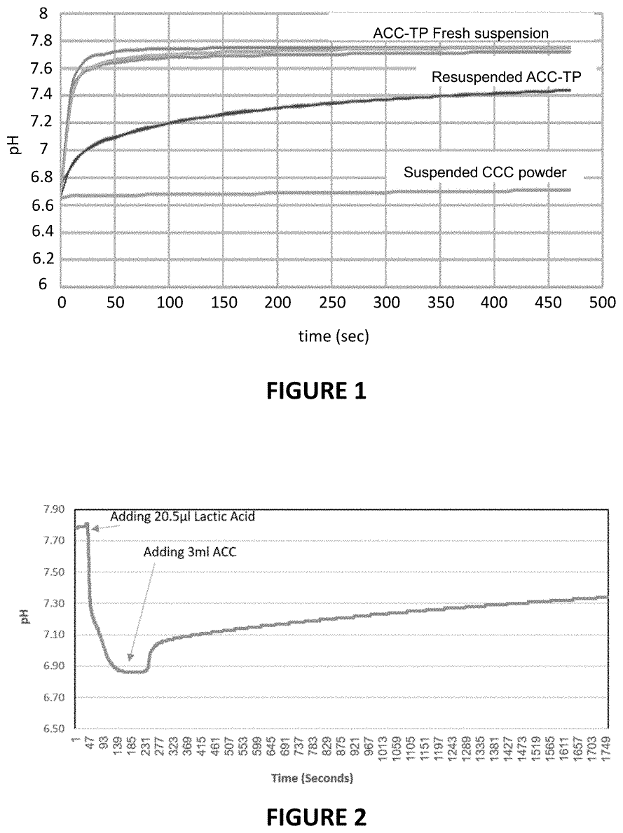

The results are presented in FIGS. 1 and 2. FIG. 1 shows the effect of ACC and CCC added to medium containing 10% serum. It can be seen that the pH was immediately reduced by adding the lactic acid. Then, ACC rapidly elevated pH from 6.6 to between 7.4 and 7.8, depending on the exact ACC formulation. The CCC did not significantly change the pH. FIG. 2 show pH measurements of the medium supplemented with 10% fetal bovine serum (FBS). The arrows indicate of the times when lactic acid and ACC solution (1% calcium, after filtration, pH 7.52—shown in FIG. 2) were added. These results demonstrate that ACC stabilized with different stabilizers (TP and CA) can elevate the pH of medium containing 10% serum up to the range of 7.4 and 7.8. These results also demonstrate that CCC does not dissolve at these pH values.

### Example 2

### Sea Horse Experiment 4T1 Result Elements

This example demonstrates how metabolism of cancer cells is converted from glycolysis, associated with acidic pH according to the “Warburg Effect”, to oxidative phosphorylation, when ACC induces a higher pH.

### Background

Mammalian cells generate ATP by mitochondrial (oxidative phosphorylation) and non-mitochondrial (glycolysis) metabolism. Cancer cells are known to reprogram their metabolism using different strategies to meet energetic and anabolic needs. This phenomenon is known as aerobic glycolysis and is one of the hallmarks of cancer cells. It means that cancer cells produce ATP through the glycolytic cycles even in the presence of oxygen. The products of glycolysis are lactate and hydrogen ion (protons), which are secreted into the intracellular compartments, resulting with an acidic microenvironment within the tumor.

### Experiment's Principle

The inventors measured the oxygen consumption rate (OCR) and extracellular acidification rate (ECAR) measurements in murine breast cancer cells (4T1) culture, utilizing a Seahorse XF24 Extracellular Flux Analyzer, which were pretreated with ACC, CCC or calcium chloride (CaCl₂), in order to evaluate their effects on the metabolic pathway of the cells.

### Materials and Methods

Seahorse assay—The Seahorse assay was performed using the XFe24 analyzer (Agilent Technologies, Santa Clara, Calif., USA). Both mitostress and glycostress assays were performed using the following reagents: oligomycin, Carbonyl cyanide-4-(trifluoromethoxy)phenylhydrazone (FCCP), antimycin, 2-deoxy-glucose.

4T1 cells (cells originated from mice mammary gland tumor) were seeded in a medium containing various calcium sources: CaCl₂—2 mM elementary calcium; ACC—2 mM elementary calcium; and CCC—2 mM elementary calcium.

### Results

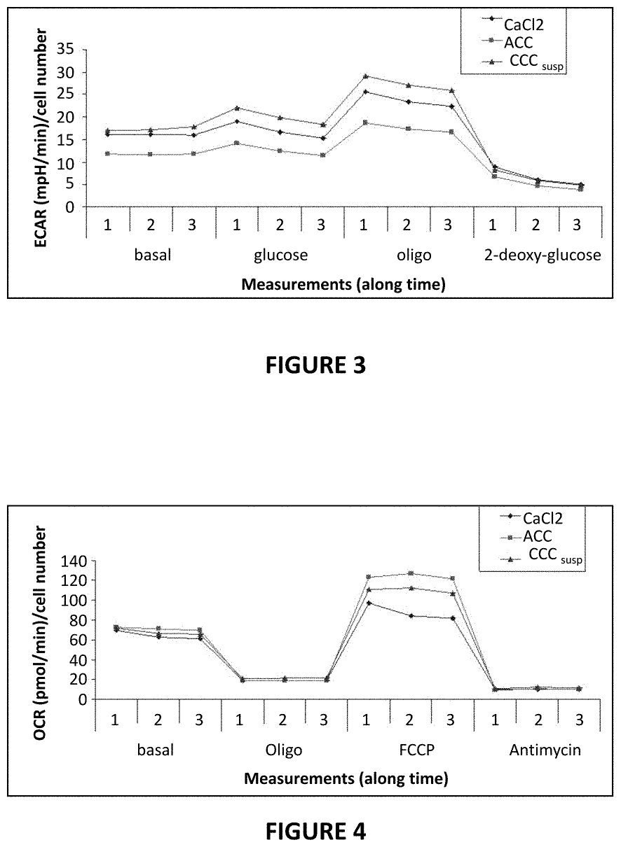

The results of the OCR levels are shown in FIG. 3 (a full seahorse run). It can be seen that ACC-treated cells had a moderate higher basal mitochondrial respiration vs. CaCl₂ and CCC. ACC-treated cells show the maximum mitochondrial respiration capacity, much higher than CaCl₂ (p. value=6.4×10⁻⁶) and CCC (p. value=0.02). These observations suggest that ACC's cancer treated cells use greater aerobic respiration than cells, which were grown in other media.

A glycostress assay was performed in order to measure the cell's glycolytic function. As seen in FIG. 3, ACC-treated cells showed much lower ECAR levels than the other two groups. It indicates a lower glycolysis rate within these cells, which correlates with the higher OCR observed in FIG. 4. This trend lasts along all the treatments and is reinforced by the ACC-treated cell response to oligomycin, i.e., the slope of the ACC line in the graph (from glucose point 3 to oligo point 1) is not as sharp as for the other cell cultures during this step, indicating a lower glycolytic activity.

In addition, the ECARs measurements after oligomycin injection are lower for ACC in comparison to the ECAR of the other treatments (see FIG. 3). The low ECAR of ACC treated cells indicates their lower glycolysis rates vs. CaCl₂ and CCC.

### Example 3

### ACC Enhances Healthy Embryo Compaction, Expansion and Hatching

This example demonstrates the effect of ACC, which increase and maintain adequate pH level, associated with the improvement of embryo development.

Faster cell proliferation was demonstrated when ACC was introduced into various culture systems in numerous experiments. These experiments also exhibited better functionality and differentiation (as seen for example, in the differentiation of mesenchymal stem cells (MSC) into osteocytes and in the contraction of muscles cells).

One of the most sensitive culture systems is the one established for embryos. The pH must be tightly controlled during in-vitro culture of mice embryos. This system is also used to evaluate the toxicity of substances, referred to as embryo assay (MEA). Many experiments demonstrated that mice embryos were developed significantly better when ACC was added to commercial embryo culture media resulting with more blastocysts (blast) and hatched blast (see Table 1).

TABLE 1
Mice embryos cultured in commercial IVF medium either
supplemented with ACC or without ACC (control).

| | Total 2PN | Compaction | | Expended Blasts | | Hatching Blasts | |
|---|---|---|---|---|---|---|---|
| | (2 | (60 hrs) | | (84 hrs) | | (110 hrs) | |
| Treatment | pronuclei) | # | % | # | % | # | % |
| Control group | 370 | 136 | 37 | 108 | 29 | 117 | 32 |
| ACC group | 440 | 281 | 64 | 252 | 57 | 347 | 79 |
| Improvement Factor | | | 1.7 | | 2.0 | | 2.5 |

The number of embryos that were developed to blasts and hatched blasts was dramatically increased in the presence of ACC. It is understood that a healthy growth requires a slightly basic pH. However, during the embryo development acidification of the medium occur, partially due to energy consumption, associated with ATP to ADP+H⁺ conversion. Therefore, the ACC serves as a solid buffer that keeps the pH at around pH 7.4 while slowly releasing essential calcium ions.

The ACC provides the very important mineral-calcium, in a slow-release fashion, associated with the neutralization of the acidity generated during the culture growth. All commercial media contain calcium, mainly by the addition of calcium chloride. Calcium is an essential ion for cell metabolism, especially for normal mitochondria functioning and the production of ATP through the oxidative phosphorylation pathway. Furthermore, the production of ATP from ADP binds a proton (H⁺) in this chemical process, hence further reducing the generated acidity.

Therefore, the inventors hypothesize that the improved results are due to a slow-release mechanism that occurs, when ACC encounters an “acidic” environment (pH below 7.4) and when the pH is elevated then the ACC stops dissolving. For example, when the medium pH is 6.6, the following reaction will occur ONLY with ACC but not with CCC:

CaCO₃₍ₛ₎+2H⁺_{(aq)}→Ca²⁺_{(aq)}+H₂CO_{3(aq)}→Ca²⁺_{(aq)}+CO_{2(g)}+H₂O₍ₗ₎

At slightly higher pH (around 7.0) bicarbonate ions (and not carbonic acid) are formed and participate in the critical bicarbonate biological buffer:

CaCO₃₍ₛ₎+H⁺_{(aq)}→Ca²⁺_{(aq)}+HCO₃⁻_{(aq)}

The evolved bicarbonate is also essential for proper metabolism in cells. Overall, ACC not only elevates the medium pH to physiological ranges but also supplement the cells with calcium.

### Example 4

### ACC Alters Gene Expressions

A series of experiments with numerous human cancer cells demonstrate that in the presence of ACC, significant changes of gene expression levels occur compared to similar concentrations of calcium derived from CaCl₂. Hence, the net effect must be associated with the increased and maintained higher pH associated with the ACC dissolution. The major gene expression changes are associated with reducing the cancer cells' capability to act as cancer cells or protect themselves against the immune system.

### General Background

As seen in Example 2, adding ACC into culture medium of cancer cells caused a shift in the cell's metabolic pathways from glycolysis, the preferred route of cancer cells, to oxidative phosphorylation, the preferred route in normal cells. The Warburg Effect, i.e., the increased glycolysis in cancer cells under aerobic conditions, was misinterpreted as evidence for respiration damage. However, it is now understood that it reflects an altered regulation of glycolysis in relation to mitochondrial function. The Warburg Effect actually comprises a complex collection of contributory shifts of gene expression and respiratory functions [Burns et al., Int J Mol Sci. 2017; 18(12). pii: E2755].

The Warburg effect is highly associated with the formation of acidosis conditions around the growing tumor, which in turn accelerates the aggressive progression of the disease. It was postulated by Warburg and later generations of researchers that combatting the acidosis conditions would suppress cancer progression.

The following experiments evaluate how adding ACC affects gene expression of various cancer cell lines, in modes that reduce their capability to evade the immune system response. The study was planned to isolate the basicity effect of the ACC by comparing the differential gene expression between ACC and calcium chloride, having equivalent molar ratio of calcium. Thus, any meaningful up- or downregulation of genes associated with cancer cells and tumor proliferation and growth can be associated with the continuous release of the basic carbonate from the ACC suspended in the culture medium.

### Materials and Methods for all Cell Lines

### Culture Medium

All materials were purchased from Biological Industries, Beit Haemek, Israel, unless specified differently.

The Spinal Cord culture medium (herein SC medium) was composed of: 90% Dulbecco's modified Eagle medium-nutrient mixture F-12 (DMEM-F12), calcium depleted, 10% (v/v) fetal bovine serum (FBS), 2 mM glutamine, Penicillin G Sodium Salt: 10,000 units/mL, Streptomycin Sulfate: 10 mg/mL (Pen/Strep).

### Thawing and Culturing:

All procedures were performed inside a laminar flow hood, under aseptic conditions. One frozen ampule of A549 cancer cells was thawed in a 37° C. water bath and seeded in two T-25 flasks (Corning Inc, NY, USA), filled with 5 ml of SC medium. The next day the medium was refreshed. Then, the medium was refreshed twice a week. After the cells reached confluency, the cells were sub-cultured in a spilt ratio of 1:5 into new T-25 flasks. The cells were split into 2 groups: (1) SC regular medium, which contains 1.05 mM calcium chloride (CaCl₂) (herein Bank). (2) SC (calcium depleted) medium with 2 mM ACC (herein Test Group). Each group consisted of three replicates (triplicate). The cultured continued for 8 passages and then part of the cells (from each group) were spanned to create a frozen bank and the other cells had their RNA isolated using Promega SV Total RNA Isolation System (Promega Corp. Madison, Wis., USA).

To avoid batch effects that may interfere with the signals in the experiments, the extraction from all nine samples was performed at the same day, time, kits, person etc.

RNA Extraction and mRNA Sequencing

The total RNA was isolated after 8 passages from all 3 groups in triplicates for each group, using Promega SV Total RNA Isolation System. The RNA integrity and concentration were detected for each sample. The mRNA libraries, sequencing and analysis were done at the Nancy and Stephen Grand Israel National Center for Personalized Medicine (G-INCPM) services at the Weisman Institute for Research in Israel. mRNA Libraries were prepared using INCPM mRNA Seq. Sequencing was done using the NextSeq SR75 high output analyzer (Star Seq GmbH, Mainz, Germany). The output comprised ˜29 million reads per sample. Single end reads (length of 84 bases) were sequenced with a sequencing depth of ˜22.2M reads per sample.

### Bioinformatics Analysis

The effects of the differential gene expression were evaluated using Ingenuity® Pathway Analysis (IPA®) software (Qiagen, Hilden, Germany)—an analysis and search tool that uncovers the significance of omics data and identifies new targets or candidate biomarkers within the context of biological systems.

The differential gene expressions were analyzed by comparing each treatment to the other as follows: ACC compared to Bank (A vs B. Results having a p adj lower than 0.05 and a fold change higher than 1.1 and a count of more than 30 per cell, were considered statistically significant.

### 4.1 Effect of ACC in A549 Lung Cancer Cells

The results of the differential gene expression of the different treatments tested on A549 cell line are summarized in Table 2 below.

TABLE 2
Differential gene expressions in A549 lung cancer
cells treated with ACC compared to untreated cells.

| | Number of | Number of | Total |
|---|---|---|---|
| | Up- | Down- | Differentially |
| | regulated | regulated | Expressed |
| Comparison | Genes | Genes | Genes |
| A549_ACC_vs_Control | 1126 | 902 | 2028 |

All these expression values were found to be statistically different (p adj<0.05). Hereinafter is a table that summarized some of the important genes, found to be differentially expressed in A549 lung cancer cell line treated with ACC.

TABLE 3
A list of important genes that were differentially expressed in A549 Lung Cancer cells, ACC
treated cells versus untreated cells, the change that was found and p values and the outcome.

| | ACC effect on | | |
|---|---|---|---|
| | gene expression | | |
| Gene name | [Fold Change | | |
| (Encoded protein) | (P Value; P adj)] | Outcome Meaning | |
| CD274 (PDL-1) | −3.8 | Activation of the immune response | |
| | (0; 0) ▾ | | |
| CYTIP (Cytohesin- | +72.5 | Activation of the immune response of T cells | |
| Interacting Protein) | (3.75 * 10⁻⁵; | | |
| | 2.62 * 10⁻⁴) ▴ | | |
| ITGB2 (CD18) | +7.2 | Activation of the immune response of T cells & NK cells. | |
| | (0; 0) ▴ | | |
| JUN (Protoonco | −3.6 | Decreasing cancer and tumorigenic pathways | |
| gene JUN) | (0; 0) ▾ | | |
| RUNX2 (RUNX2 or | −15.35 | 1. | Decreasing tumor spreading. |
| | (0; 0) ▾ | 2. | Activation of cancer cells apoptosis. |
| CBF- alpha - 1) | | | |
| CD244/2B4 (CD244) | −227.54 | Activation of the immune response of cytotoxic T cells & | |
| | (0.00441; | NK cells. | |
| | 0.0191) ▾ | | |
| TGFB1—(TGF β) | −1.6 | 1. | Inhibition of angiogenesis. |
| | (0; 0) ▾ | 2. | Activation of the Immune response (Inhibition of the |
| | | | Immune-escape). |
| | | 3. | Inhibition of Metastasis |
| | | 4. | Inhibition of epithelial normal cells apoptosis. |

| | | |
|---|---|---|
| TMSB4X (Thymosin | −1.67 | Decreasing tumor metastasis and angiogenesis |
| beta4) | (0; 0) ▾ | |
| SNAI2/SLUG—Snail | −3.758 | ACC decreases the expression of SNAI2/SLUG [symbol] Reducing |
| Family Transcriptional | (0.0031; | tumor growth and progression. |
| Repressor 2) | 0.014) ▾ | |

The gene CD274 encodes the protein PDL-1, which is an overexpressed protein in cancer cells. Immune checkpoint pathway is a focal point of today's cancer research. PD-1 is one of the best characterized checkpoint proteins. The binding between PD-1 receptor and its ligand PD-L1 suppresses T-cell activation and allows cancer cells to escape from the body's immune surveillance. Therefore, reducing PD-1 pathway activity has been considered a promising anti-cancer treatment.

Surprisingly, the results in this study show that the addition of ACC to the culture medium downregulated the expression of this gene in a fold change (FC) of 3.8. ACC is specific by nature since it will dissociate only in an acidic environment, like that found in solid tumors.

Therefore, it downregulates the expression of PDL-1 mainly in the cancer cells, restoring the antitumor immune response.

The gene TGFB1, which encodes the protein TGFβ1 was downregulated by 1.6 FC. The JUN gene, which encodes the protooncogene JUN was downregulated when treated with ACC, by FC of 3.6. JUN is a proto-oncogene & transcription factor, known to increase cancer & tumorigenesis pathways. Downregulation of this gene leads to decreasing in cancer and tumorigenesis.

A549 cells treated with ACC demonstrated an up-regulation of 7.21 FC for ITGB2 integrin subunit β2 gene expression, P-Adj Value—0. The ITGB2 gene encodes CD18 protein. The above results indicate that the ACC activates the immune system respond.

The gene RUNX2 is another important gene, which is affected by the ACC. In A549 lung cancer cells, RUNX2 gene was downregulated in a FC of −15.348, (P-Adj and P. Value—0) in the presence of ACC. The gene 2B4 (CD244) was downregulated in FC of −227.54 (p-Value 0.00441; p-Adj 0.0191), by adding the ACC. The gene TMSB4X (Thymosin beta4) was downregulated by FC of −1.67 (P-Value 0; p-Adj 0), by ACC. The gene SNAI2/SLUG, which encodes the protein Snail Family Transcriptional Repressor was downregulated by FC of −3.758 (P-Value 0.0031; p-Adj 0.014), by the ACC. This decrease in the expression leads to inhibition of the cancer progression and invasion.

The outcome of these results demonstrates that treatment with ACC leads to decreased cancer pathways, lowering the ability of the cancer cells to invade and spread metastases. Furthermore, the ACC affected checkpoint genes which are target genes for immunotherapy.

Additional experiments (see below) demonstrated reduction of Cathepsin B activity and a reduction in lung cancer tumors size in mice. These results shed light on the activity mechanism of ACC. High levels of Cathepsin B are associated with tumor invasion and metastasis. Since Cathepsin B activity is induced by an acidic environment, the inhibition of its activity is associated with the continuous neutralization obtained by the ACC. This experiment shows that ACC causes a significant differential gene expression, which leads to antitumorigenic effects by up/down regulating genes that allow the immune system to better attack the tumor, reduce proliferation, invasion, and metastatic pathways, causing an overall anti-cancer effect.

### 4.2 Effects of ACC on LNCaP Clone FGC Prostate Carcinoma Cancer Cells

### Results

The effect of ACC on LNCaP human prostate carcinoma cancer cells, (Test Group vs. control 1/Bank) showed that 64 genes were differentially expressed in a statistically significant manner: 16 genes were upregulated and 48 were downregulated (Table 4), with thresholds of absolute FC of at least 1.5, adjusted p-value below 0.05, and a count of at least 30 in one of the samples. Meaningful genes, which were differentially expressed (DE) with the addition of the ACC, are listed in Table 5, in which the resultant protein role and function, expression levels in cancer cells and the outcome of the change in the expression are stated. The effect of some genes, which were downregulated, was decreasing cancer and tumorigenic pathways. Part of the affected genes are known as Immune target checkpoints and are used by the cancer cells to inhibit the immune system. Downregulation of these genes or blocking them with antibodies can activate the immune system and contribute to decreasing the spread of the cancer.

TABLE 4
Differential gene expression in LNCaP human prostate carcinoma cancer
cells with ACC treatment versus control non treated cells.

| | Number of | Number of | Total |
|---|---|---|---|
| | Up- | Down- | Differentially |
| | regulated | regulated | Expressed |
| Comparison | Genes | Genes | Genes |
| LNCaP_ACC vs Control | 16 | 48 | 64 |

TABLE 5
A list of important genes that were differentially expressed in LnCap Prostate
Cancer cells

| | ACC effect on | |
|---|---|---|
| | gene expression | |
| Gene name | [Fold Change | |
| (Encoded protein) | (P Value; P adj)] | Outcome |
| TMSB4X—Thymosin | −9.9176 | Decreasing tumor metastasis and angiogenesis. |
| beta4 | (1.86 *10⁻⁴; | |
| | 0.0155) ▾ | |
| SNAI2/SLUG—(Snail | −2.3456 | ACC decreases the expression of SNAI2/SLUG |
| Family Transcriptional | (0.0000172; | [symbol] Reducing tumor growth and progression. |
| Repressor 2) | 0.00238) ▾ | |
| CD44 (CD44) | −7.727 | ACC decreases the expression of CD44 |
| | (0.000619; | [symbol] Decreasing all 4 cancer pathways. |
| | 0.0398) ▾ | |
| CD 74 | −14.025 | ACC decreases the expression of CD74 |
| | (0.000497; | [symbol] Decreasing cancer cell proliferation, invasion, |
| | 0.0344) ▾ | and angiogenesis. |
| ETV1—(ETS | −3.605 | Decreasing tumor metastasis, invasiveness, and |
| (E twenty-six) family | (0; 0) ▾ | malignant progression. |
| of transcription factors). | | |
| FGFR3—(Fibroblast | −1.5178 | ACC decreases the expression of FGFR3 |
| Growth Factor | (0.000531; | [symbol] Reducing cancer cell proliferation and progression. |
| Receptor 3) | 0.0358) ▾ | |

Table 5 shows a list of important genes that were differentially expressed in the presence of ACC versus untreated cells. The role of each gene in cancer is described including the change that was found and p values and the outcome (i.e., the interpretation) of the changes on decreasing cancer pathways or activation of the immune response against the cancer cells.

The gene CD44 encodes the CD44 antigen, which is a cell-surface glycoprotein involved in cell-cell interactions, cell adhesion and migration. In this study CD 44 was downregulated by ACC in a fold change (FC) of −7.727 (P-value 0.000619; P-adj 0.0398).

The gene CD 74 encodes protein CD 74, which is a non-polymorphic glycoprotein that has diverse immunological functions. The results in this study demonstrated that ACC downregulated the expression of CD74 in a FC of −14.025 (P-value 0.000497; P-adj 0.0344).

The gene SNAI2/SLUG encodes the protein Snail Family Transcriptional Repressor. ACC downregulated the gene SNAI2/SLUG in FC of −2.3456 (P-Value 0.0000172; p-Adj 0.00238). Slug belongs to the Snail family and is a well-known EMT-inducing transcription factor/E-cadherin transcriptional repressor. Slug has been implicated in tumor development and the progression of prostate cancer based on its elevated expression compared with that of other family members. This downregulation inhibits cancer cell sphere formation, invasion, and suppresses tumor regeneration and metastasis. The downregulation of SNAI2/SLUG together with downregulation of CD44 & CD74 affect important pathways and will lead to inhibition and suppressing of prostate cancer.

TMSB4X—Thymosin Beta 4 X-Linked gene encodes Thymosin Beta4 protein, which plays a role in regulation of actin polymerization. The protein is also involved in cell proliferation, migration, and differentiation. In LNCaP human prostate carcinoma cancer cells this gene was downregulated, in FC of −9.9176 (P-value 1.86*10-4 P adj. 0.0155), by ACC. These results indicate that using ACC by prostate cancer patients will inhibits tumor metastasis, invasion, and angiogenesis.

ETV1 gene encodes the protein ETS—E twenty-six family of transcription factors. In LNCaP human prostate carcinoma cancer cells this gene was downregulated by FC of −3.605 (P-value 0; P adj. 0), by the ACC. Downregulation of ETV1 results in decreasing tumor metastasis, invasiveness, and malignant progression.

FGFR3 gene encodes the protein Fibroblast Growth Factor Receptor 3, which is a member of the fibroblast growth factor receptor (FGFR) family. This gene was downregulated, by FC of −1.5178 (P-value 0.000531 P adj. 0.0358), by the ACC.

The obtained results suggest that ACC has an antitumorigenic effect on prostate cancer cells. The ACC changes the gene expressions of genes that are involved in promoting the tumor via proliferation, invasion, metastasis, and immune system evasion. Affecting these genes may be one of the causes for the capability of the basic ACC to serve as a therapeutic agent for healing prostate cancer by altering the acidosis conditions.

4.3 Effects of ACC on HCT116 Colorectal Carcinoma cells

The effect of ACC on HCT116 Colorectal Carcinoma Colon cancer cells, (Test Group vs. control 1/Bank), showed that 54 genes were upregulated and 18 were downregulated (Table 6), with thresholds of absolute FC of at least 1.5, adjusted p-value below 0.05 and a count of at least 30 in one of the samples. Meaningful genes, which were differentially expressed (DE) by the addition of ACC are listed in Table 7, the protein role and function, expression levels and the outcome of the change in the expression are stated.

TABLE 6
Differential gene expression in HCT116 Colorectal
Carcinoma Colon cancer cells, treated with ACC
versus cells Treated with calcium chloride.

| | Number of | Number of | Total |
|---|---|---|---|
| | Up- | Down- | Differentially |
| | regulated | regulated | Expressed |
| Comparison | Genes | Genes | Genes |
| HCT116_ACC vs Control | 54 | 18 | 72 |

TABLE 7
Differential expression of genes in HCT116 Colorectal Carcinoma
Colon cancer cells, ACC treated cells versus untreated cells,
the rule of the genes in cancer cells and the outcome

| | ACC effect on | |
|---|---|---|
| | gene expression | |
| Gene name | [Fold Change | |
| (Encoded protein) | (P Value; P adj)] | Outcome |
| GDF15—(growth | −1.52 | ACC decreases the expression of GDF15 |
| differentiation | (0; 0) ▾ | [symbol] Decreasing colorectal cancer invasion |
| factor 15) | | and metastasis. |
| | | Decreasing NHST |
| TGFA—(transforming | −1.18 | ACC decreases the expression of TGFA |
| growth factor alpha) | (0.00143; | [symbol] Decreasing cancer cell proliferation, |
| | 0.0427) ▾ | osteoclastic bone resorption. |
| | | Decreasing NHST. |
| SUSD2 (Sushi Domain | + 1.826 | ACC increases the expression of SUSD2 |
| Containing 2) | (0; | [symbol] suppressing tumorigenic phenotypes. |
| | 2.2 × 10⁻⁹) ▴ | Decreasing cancer cell proliferation & |
| | | invasiveness. |

GDF15 gene encodes the Growth/differentiation factor 15 (GDF15) protein. In HCT116 Colorectal cancer cell line this gene was downregulated, by FC of −1.52 (P-value 0 P adj. 0) by ACC. This gene encodes a secreted ligand of the TGF-beta (transforming growth factor-beta) superfamily of proteins. Increased levels of GDF15 are associated with colorectal cancer invasion, metastasis, and poor prognosis. ACC decreases the expression of GDF15 leading to decreasing in colorectal cancer invasion, metastasis.

TGFA gene encodes the transforming growth factor alpha protein and it was downregulated, by FC of −1.18 (P-value 0.00143; P adj. 0.0427), by ACC. This gene encodes a growth factor that is a ligand for the epidermal growth factor receptor, which activates a signaling pathway for cell proliferation, differentiation, and development. TGFA stimulate proliferation of colon cancer cells, but not colon normal cells.

SUSD2 gene encodes the Sushi domain-containing protein 2, and it was upregulated by FC of +1.826 (P-value 0; P adj. 2.2×10⁻⁹) in the presence of ACC. SUSD2 may be a tumor suppressor, together with chromosome 10 open reading frame 99 (C10orf99) has a growth inhibitory effect on colon cancer cells which includes G1 cell cycle arrest.

### 4.4 Effects of ACC on MCF7 Human Breast (Adenocarcinoma) Cancer Cells

The study examined the effect of ACC on the gene expression of MCF7 human breast (adenocarcinoma) cancer cells in comparison to Non treated (1.05 mM Ca⁺²) cells and to 2 mM Ca⁺² originated from CaCl₂ cells. This cell line (MCF7) is of a human ER⁺ and PR⁺ breast cancer type.

The ACC presence in cultures of MCF7 human breast (adenocarcinoma) cancer cells (Test Group vs. control 1/Bank) caused 66 genes to be differentially expressed in a statistically significant manner: 22 genes were upregulated and 44 were downregulated (Table 8), with thresholds of absolute FC of at least 1.5, adjusted p-value below 0.05 and a count of at least 30 in one of the samples.

Meaningful genes, which were differentially expressed (DE) with the addition of ACC are listed in Table 8, the protein role and function, expression levels in cancer cells and the outcome of the change in the expression are stated in Table 9.

TABLE 8
Differential gene expression in MCF7 human breast
(adenocarcinoma) cancer cells, treated with ACC
versus cells treated with calcium chloride.

| | Number of | Number of | Total |
|---|---|---|---|
| | Up- | Down- | Differentially |
| | regulated | regulated | Expressed |
| Comparison | Genes | Genes | Genes |
| MCF7_ACC vs | 22 | 44 | 66 |
| Control 1/Bank | | | |

TABLE 9
Differential expression of genes in MCF7 human breast (adenocarcinoma) cancer
cells ACC treated cells versus untreated cells, the outcome of the changes
on decreasing and suppressing tumorigenicity and cancer pathways.

| | ACC effect on | | |
|---|---|---|---|
| | gene expression | | |
| Gene name | [Fold Change | | |
| (Encoded protein) | (P Value; P adj)] | Outcome | |
| SAMD9—(Sterile | +2.828427125 | ACC increases the expression of SAMDA9 | |
| Alpha Motif Domain- | (0.000249; | [symbol] Overexpression of SAMD9 suppresses | |
| containing 9) | 0.0175) ▴ | tumorigenesis and progression during non- | |
| | | small cell lung cancer. | |
| OAS1 (Oligo | +2.3133 | ACC increases the expression of OAS1 | |
| Adenylate | (0; 0) ▴ | 1. | [symbol] Overexpression of OAS1 suppresses |
| Synthetase 1) | | | the progression of breast cancer. |
| | | 2. | Enhances the immune response. |

| | | |
|---|---|---|
| OAS2 (Oligo | +2.514 | ACC increases the expression of OAS2 |
| Adenylate | (0.0000189; | [symbol] Enhances the immune response. |
| Synthetase 2) | 0.00219) ▴ | |
| GSTM2 (Glutathione | −2.15845647 | ACC decreases the expression of GSTM2 |
| Transferases) | (0.0000342; | [symbol] Decreasing the chemoresistance of the |
| | 0.00358) ▾ | cancer cells. |

SAMD9 gene encodes the Sterile Alpha Motif Domain-containing 9 (SMAD 9) protein. The gene was upregulated, by FC of +2.828427125 (P-value 0.000249 P adj. 0.000249), in the presence of ACC. The Sterile Alpha Motif Domain-containing 9 (SAMD9) gene has been recently emphasized after discovering that it is expressed at a lower level in aggressive fibromatosis and some cases of breast and colon cancer.

The OAS1 and OAS2 genes encode the Oligoadenylate synthase-like 1 and 2 proteins, respectively. OAS1 was upregulated by FC of +2.3133 (P-value 0 P adj. 0), by the ACC. OAS2 was upregulated by FC of +2.514 (P-value 0.0000189 P adj. 0.00219) with ACC. Oligo Adenylate Synthetase 1 (OAS1) and OAS2 are interferon-induced proteins, characterized by their capacity to catalyze the synthesis of 2′-5′-linked oligomers of adenosine from adenosine triphosphate (2-5A). Overexpression of OAS1 suppresses the progression of breast cancer and both OAS1 & OAS2 enhances the innate immune response.

GSTM2 gene encodes the Glutathione S-Transferase Mu 2 (GSTM2) protein. This gene was downregulated by FC of −2.15845647 (P-value 0.0000342 P adj. 0.00358) by ACC.

ACC treatment down regulated, GSTM2 gene in FC of −2.15845647 leading to suppression of drug resistance assisting therapeutic efficacy.

These results demonstrate that treatment with ACC leads to decreasing in cancer resistance and lowering the ability of the cancer cells to proliferate, invade and metastasize. These findings together with parallel results that demonstrate the effect of ACC over murine breast cancer (4T1) and human breast Mammary Gland cells (see Examples 4.5 and 4.9) suggest that ACC may be involved in multiple anticancer effects, associated with the generation of the basic conditions.

### 4.5 Effects of ACC Over MDA-MB-231 Human Breast Mammary Gland (Adenocarcinoma) Cancer Cells

The study examined the effect of ACC on gene expression of MDA-MB-231 human breast Mammary Gland (adenocarcinoma) cancer cells, in comparison to Non 1.05 mM Ca⁺² treated cells (originated from CaCl₂).

The effects of ACC on MDA-MB-231 human breast Mammary Gland (adenocarcinoma) cancer cells is manifested by 19 genes that were differentially expressed in a statistically significant manner: 11 genes were upregulated and 8 were downregulated (see Table 10), with thresholds of absolute FC of at least 1.5, adjusted p-value below 0.05 and a count of at least 30 in one of the samples.

Meaningful genes, which were differentially expressed (DE) with the addition of ACC are listed in Table 11, including the protein rule and function, expression levels in cancer cells and the outcome of the change in the expression are stated. The effects of the downregulated genes lead to decreasing cancer and tumorigenic pathways.

TABLE 10
Differential gene expression in MDA-MB-231 human breast
Mammary Gland (adenocarcinoma) cancer cells, treated
with ACC versus cells treated with calcium chloride.

| | Number of | Number of | Total |
|---|---|---|---|
| | Up- | Down- | Differentially |
| | regulated | regulated | Expressed |
| Comparison | Genes | Genes | Genes |
| MDA-MB-231_ACC vs | 11 | 8 | 19 |
| Control 1/Bank | | | |

TABLE 11
Differential expression of genes in MDA-MB-231 human breast Mammary
Gland (adenocarcinoma) cancer cells, ACC versus CaCl₂ treated cells

| | ACC effect on | | |
|---|---|---|---|
| | gene expression | | |
| Gene name | [Fold Change | | |
| (Encoded protein) | (P Value; P adj)] | Outcome | |
| ALDH1A1 (Aldehyde | −93.0542411 | ACC decreases the expression of ALDH1A1 [symbol] | |
| dehydrogenase) | (0.000219; | 1. | Overexpression of ALDH1A1 increased |
| | 0.01980) ▾ | | the resistance to drugs and radiation. |
| | | 2. | Decreasing the expression of ALDH1A1 |
| | | | leads to higher response to drugs and |
| | | | radiation. Improving cure chances. |
| CNTN1 | −2.73208051 | ACC decreases the expression of CNTN1 [symbol] | |
| | (2.4E−09; | 1. | Overexpression of CNTN1 promoted |
| | 0.0000022) ▾ | | cell proliferation, cell cycle progression, |
| | | | colony formation, invasion, and migration. |
| | | 1. | Decreasing the expression of CNTN1 |
| | | | leads to suppression in cancer cell |
| | | | proliferation, invasions, and progression. |

| | | |
|---|---|---|
| ESAM (Endothelial | −5.65685425 | ACC decreases the expression of ESAM [symbol] |
| cell-selective | (1.93E−08; | Decreasing the expression of ESAM leads to |
| adhesion molecule) | 0.0000124) ▾ | decreasing in: |

| | |
|---|---|
| 1. | Endothelial cell migration. |
| 2. | Tube formation. |
| 3. | Angiogenesis. |

| | | |
|---|---|---|
| ESMI (Endothelial | −2.37 | ACC decreases ESMI expression, this is |
| cell specific | (2.4E−09; | associated with better prognosis and higher |
| molecule-1) | 0.00000224) ▾ | relapse-free rates. |

ALDH1A1 gene encodes Aldehyde dehydrogenase 1 family, member A1. This gene was downregulated, by FC of −93.0542411 (P-value 0.000219 P adj. 0.0198) in the presence of ACC.

CNTN1 gene encodes the Contactin1 protein. This gene was downregulated by FC of −2.73208051 (P-value 2.4E-09; P adj. 0.0000022) with ACC. ESAMV gene encodes Endothelial cell-selective adhesion molecule. Endothelial cell-selective adhesion molecule (ESAM) is a member of the immunoglobulin receptor family that mediates homophilic interactions between endothelial cells. This gene was downregulated, in FC of −5.65685425 (P-value 1.93E-08; P adj. 0.0000124) in the presence of ACC.

ESM1 (Endothelial cell specific molecule-1) gene encodes a protein called Endocan. ESM1 is a 50 kDa soluble proteoglycan, which is frequently overexpressed in many cancer types. This gene was downregulated in a FC of −3.37 (P-value 2.4E-09; P adj. 0.00000224).

The outcome of these results demonstrates that treatment with ACC leads to the decrease in cancer capabilities, lowering the ability of the cancer cells to invade, develop solid tumor and causing metastases.

### 4.6 Effects of ACC on HELA Human Adenocarcinoma Cervix Cancer Cells

One of the deadliest cancers since it is detected after the cancer has metastasized in most cases. This study examined the effect of ACC on the gene expression of HELA human Adenocarcinoma Cervix cancer cells in comparison to untreated cells (1.05 mM Ca⁺² originated from CaCl₂).

The effect of ACC over HELA human Adenocarcinoma Cervix cancer cells is manifested by 143 genes that were differentially expressed in a statistically significant manner: 113 genes were upregulated and 30 were downregulated (see Table 12), with thresholds of absolute FC of at least 1.5, adjusted p-value below 0.05 and a count of at least 30 in one of the samples.

Meaningful genes, which were differentially expressed (DE) with the addition of the ACC, are listed in Table 13, indicating the protein role and function, expression levels in cancer cells and the outcome of the expression change. The effects of downregulated genes are in decreasing cancer and tumorigenic pathways (Table 13).

TABLE 12
Differential gene expression in HELA human Adenocarcinoma Cervix
cancer cells, treated with ACC versus calcium chloride.

| | Number of | Number of | Total |
|---|---|---|---|
| | Up- | Down- | Differentially |
| | regulated | regulated | Expressed |
| Comparison | Genes | Genes | Genes |
| HELA human | 113 | 30 | 143 |
| Adenocarcinoma | | | |
| Cervix cancer_ACC vs | | | |
| Control 1/Bank | | | |

TABLE 13
Differential expression of genes in HELA human Adenocarcinoma
Cervix cancer cells treated with ACC versus CaCl₂.

| | ACC effect on | |
|---|---|---|
| | gene expression | |
| Gene name | [Fold Change | |
| (Encoded protein) | (P Value; P adj)] | Outcome |
| DMBT1(Deleted in | +2.2 | ACC increases DMBT1 resulting with an |
| malignant brain | (0.; 0) ▴ | inhibition of proliferation, migration, and |
| tumors 1) | | invasion. |
| VIT (Vitrin) | −2.2 | ACC reduces the expression of VIT which will |
| | (2E−10; | most likely reduce the tumor's ability to evade |
| | 2.84E−08) ▾ | the immune system. |

DMBT1 belongs to the scavenger receptor cysteine rich superfamily, which is mainly expressed in epithelial cells, and its variants have been demonstrated to play roles in resisting bacterial and viral infections, regulating inflammation, and affecting epithelial and/or stem cell differentiation. This gene was upregulated by a FC of +2.2 (P-value 0; P adj. 0).

VIT encodes the protein Vitrin. This gene encodes an extracellular matrix (ECM) protein.

Treated with ACC this gene was downregulated in a FC of −2.2 (P-value 2×10⁻¹⁰; P adj. 2.84×10⁻⁸).

The outcome of these results demonstrated that treatment with ACC leads to decreasing in cancer capabilities, lowering the ability of the cancer cells to invade, metastases and evade the immune system.

### 4.7 Effects of ACC on KG1A Human Acute Myelogenous Leukemia, Promyeloblast, Macrophage Bone Marrow Cancer Cells.

The study examined the effect of ACC over gene expression of KG1A human acute myelogenous leukemia cancer cells in comparison to cells treated with 1.05 mM Ca⁺², originated from CaCl₂.

The presence of ACC in a culture of KG1A human acute myelogenous leukemia cancer cells led to 78 genes that were differentially expressed in a statistically significant manner: 27 genes were upregulated and 51 were downregulated (see Table 14), with thresholds of absolute FC of at least 1.5, adjusted p-value below 0.05 and a count of at least 30 in one of the samples.

Meaningful differentially expressed (DE) genes with the addition of ACC are listed in Table 15 that include the protein role and function, expression levels in cancer cells and the outcome of the change in the expression are stated.

TABLE 14
Differential gene expression in KG1A human acute
myelogenous leukemia cancer cells, treated with
ACC versus cells treated with calcium chloride.

| | Number of | Number of | Total |
|---|---|---|---|
| | Up- | Down- | Differentially |
| | regulated | regulated | Expressed |
| Comparison | Genes | Genes | Genes |
| KG1A human acute | 27 | 51 | 78 |
| myelogenous | | | |
| leukemia_ACC vs | | | |
| Control 1/Bank | | | |

TABLE 15
Differential expression of genes in KG1A human acute myelogenous leukemia
cancer cells treated with ACC versus CaCl₂ treated cells.

| | ACC effect on | |
|---|---|---|
| | gene expression | |
| Gene name | [Fold Change | |
| (Encoded protein) | (P Value; P adj)] | Outcome |
| IL1RN (Interleukin-1 | +2.37 | ACC elevates IL1RN expression leading to an anti- |
| receptor antagonist) | (0.000000294; | carcinogenic effect. |
| | 0.0000483) ▴ | |
| S100P (S100 calcium | −2.48 | ACC downregulates A100P, which reduces tumor |
| binding protein P) | (0.00000579; | proliferation and invasion as a drug resistant cell. |
| | 0.000624) ▾ | |
| DDIT4 gene (DNA- | −2.014 | ACC downregulates DDIT4, which reduces tumor |
| damage-inducible | (0; | proliferation and invasion. |
| transcript 4) | 1E−10) ▾ | |
| JUN (c-JUN) | −3.22 | Targeting JUN may result with a therapeutic benefit |
| | (0.0000649; | in AML |
| | 0.00469) ▾ | |

IL1RN encodes the protein Interleukin-1 receptor antagonist. This gene was upregulated in a FC of +2.37 (P-value 0.000000294; P adj. 0.0000483). S100P encodes S100 calcium-binding protein P. DDIT4 gene—encodes DNA-damage-inducible transcript 4 (DDIT4) protein also known as protein regulated in development and DNA damage response 1 (REDD1). This gene was downregulated by a FC of −2.48 (P-value 0.00000579; P adj. 0.000624).

JUN—encodes the c-JUN protein. c-Jun, in combination with c-Fos, forms the AP-1 early response transcription factor.

### Example 5

### Effects of ACC on Tumor Growth Rate and Cathepsin B Activity in a Sub-Cutaneous Model of Lewis Lung Carcinoma in Mice

Cathepsin B enhanced activity is well known to be associated with the progress of tumors. It is also known to be active in acidic conditions that are generated around the tumor. This in-vivo experiment connect the ACC activity and its higher pH effect to (a) reducing the activity of Cathepsin B in tandem with (b) the reduction of tumor growth rate. These pH effects are in addition to any anticancer activity and/or immunity system improvement that is attributed to efficient bioactivity of calcium ion.

### Aim

Cathepsin B is the most studied Cathepsin type in cancer metastasis. It is active in acidic pH and significantly less active when the pH is increased to the normal range. Without being limited to any particular theory, it is assumed that as the alkalinity of ACC suspensions affects the pH of the tumor microenvironment. Additional hypothesis for the reduction in tumor size and the effect on Cathepsin B activity may be related to the effect of ACC on the immune system.

The purpose of these studies was to examine the basicity effect of ACC on subcutaneously injected Lung Lewis carcinoma tumor growth, and measure Cathepsin B activity in the tumor microenvironment. Cathepsin B is known to be activated in acidic environment, such as the acidic conditions generated by tumors, and it assists in the out growing of tumors. Two experiments were performed. In the first experiment, the tumor growth progression was examined for a period of 11 days starting from treatment administration, and the Cathepsin B levels were determined at study termination in comparison to control animals that received only saline. In the second experiment, the tumor's growth rates after ACC administration were compared to saline (negative control), Cisplatin (a chemotherapy drug as positive control) and the combination of ACC and Cisplatin.

### Materials and Methods

The first study was performed on C57BL/6 mice aging from 6 to 8 weeks. In the first experiment sixteen (16) mice, allocated into 2 groups, were used. Light anesthesia of the C57BL/6 mice was achieved by isoflurane inhalation. LLC cells in a concentration of 2.5×10⁵ cells in 100 μl PBS were injected intradermal (subcutaneously) into the right flank of each mice. Ten days following tumor inoculation, when the tumor reached a volume >60 mm³, the mice were randomly divided into study groups.

Group A has received 0.2 ml ACC with 0.5% (wt/v) of elemental calcium suspension that was injected intraperitoneal (IP) twice a day, 7 days a week. Group B received 0.2 ml saline as a negative control IP, twice a day, 7 days a week. The treatments were administered to each group from Day 10 onwards. Treatment duration lasted for 11 days.

Cathepsin B activity was measured for the group administered with ACC (0.5% w/v Ca) and compared with the group administered with Saline, the negative control. 8 tumors were analyzed per group. For the assay, 20 mg from each extracted tumor were weighed and lysed. Measurements were done using Cathepsin B Activity Assay Kit (Fluorometric) (ab65300) by Abcam (Cambridge, UK). The assays were performed on tumors from the two groups. The lysed samples were treated per Kit instructions, i.e., incubated with Cathepsin B substrate for 1 hour at 37° C. to identify the activity of the lysosomal protein.

In the second experiment, C57BL/6 mice at the age of 6 to 8 weeks were used. Light anesthesia of the C57BL/6 mice was achieved by isoflurane inhalation. LLC cells at a concentration of 2.5×10⁵ cells in 100 μl PBS were injected intradermally (subcutaneously) into the right flank of each mice. Ten days following tumor inoculation, when the tumor reached a volume of >60 mm³, mice were randomly allocated into 4 groups that received the following treatments: Group (1) received 0.2 ml ACC suspension with 0.5% (wt/v) of elemental calcium that was injected intraperitoneally (IP) twice a day, 7 days a week; Group (2) received 0.2 ml Saline injections (IP) twice a day, 7 days a week; Group (3) was injected with Cisplatin—according to manufacturer instruction (IP injection twice a week); Group (4) received a combination of ACC and Cisplatin (ACC dose the same as in Group (1) and Cisplatin same as given to Group (3). Each group consisted of 8 mice (total of 32 mice). Treatments were given for a duration of 14 days and the tumors were measured every second day. Tumor volumes were calculated according to the following formula: (length×width²)/2 where length represents the largest tumor diameter, and width represents the smallest tumor diameter.

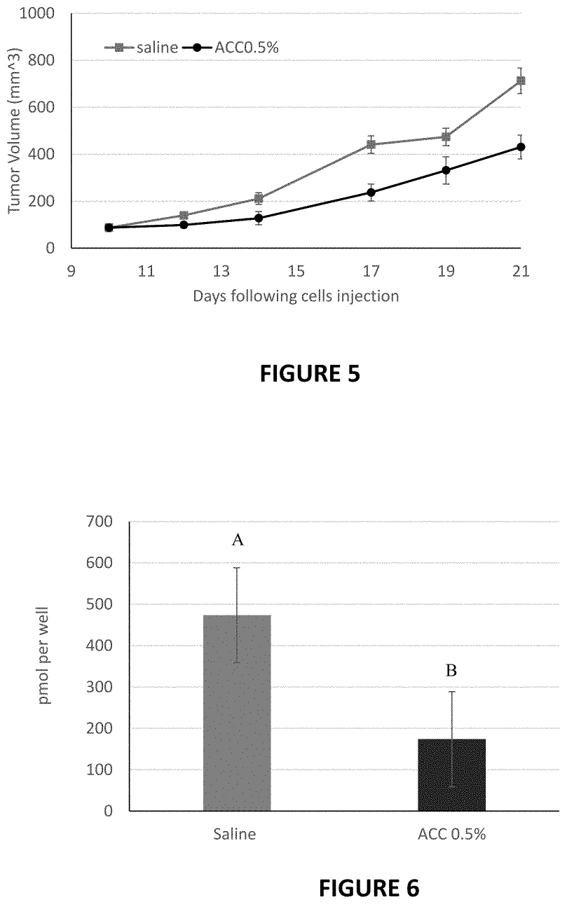

The graph in FIG. 5 clearly indicates that ACC leads to reduced tumor size, compared to the negative control. Moreover, Cathepsin B activity taken from mice treated with ACC was significantly lower than the Cathepsin activity in tumors of the untreated group with fractional activity ratio of 0.38 (see FIG. 6).

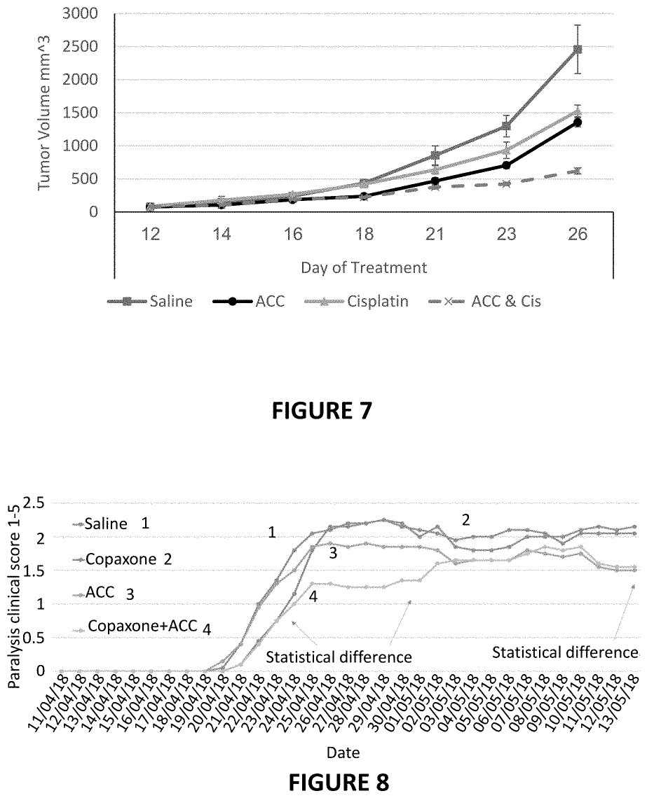

FIG. 7 shows that mice treated by ACC had reduced tumor size compared to saline, in a similar manner to the cytotoxic effect of cisplatin. Moreover, the combination of ACC and Cisplatin resulted in a synergetic effect on the tumor growth deceleration.

The outcome of lower Cathepsin B activity in the group that was treated with ACC together with the finding that this treatment resulted in a deceleration of tumor growth leads to the assumption that ACC affects the microenvironment pH of the tumor, resulting with an antitumorigenic effect as seen by the decrease in the tumor volume, compared to negative control animal.

### Example 6

### Multiple Sclerosis Model in Mice Treated with ACC Alone or in Combination with Copaxone

Multiple sclerosis (MS) is a primary inflammatory demyelinating disease associated with a secondary progressive neurodegenerative component. Impaired mitochondrial functioning has been hypothesized to drive neurodegeneration and cause increased anaerobic metabolism in MS. In a multicenter clinical study, it was found that lactate serum levels were three times higher in MS patients than in healthy people. Our hypothesis is that ACC will show a beneficial effect on animals due to its pH modulation ability, which results in an anti-inflammatory effect by increasing the pH of the local acidosis environment. As seen in another given example Cathepsin B activity in mice with an induced MS model was lower in animals treated with ACC compared to control (negative control, saline).

The mice experimental Autoimmune Encephalomyelitis (EAE) model is an accepted model for human Multiple Sclerosis (MS). In the course of MS, an autoimmune response damages the myelin sheath. This damage disrupts the ability of parts of the nervous system to communicate, resulting in a wide range of signs and symptoms including changes in sensation, muscle weakness, movement and balance difficulties, speech and swallowing impairment, vision loss, fatigue etc.

This study evaluates the efficacy of ACC on MS induced in mice. This model inhibits the Th1 cytokine response, which is in the base of the inflammatory response.

### Materials and Methods

### Test Items and Control Administration

Myelin oligodendrocyte glycoprotein (MOG) 35-55 solution was freshly prepared prior to each inoculation session by dissolving the RP-HPLC-purified lyophilized powder in PBS to achieve a solution at a final injected concentration of 2 mg/ml mixed 1:1 with CFA [Complete Freund's Adjuvant (CFA) suspension, containing killed Mycobacterium Tuberculosis H37 R at a concentration of 4 mg/ml]. aliquots of 200 microliter of the solution were injected subcutaneously (SC) into the flank of the mice.

### Treatment

The study consisted of four groups:
1. Group 1F was the negative control and was administered with vehicle only. That is Saline twice per day, administrated IP from day “10” onwards until study terminate;
2. Group 2F was a comparative control and was administered with Copaxone. This group was treated per drug instruction, i.e., once a day from day “0” until day “8”;
3. Group 3F was administered IP with ACC suspension twice per day from day “10” onwards until study terminate;
4. Group 4F was administered with both ACC suspension and Copaxone. This group examined the synergic effect of the two combines. Initially, from day “0” until day “8” the group was treated with Copaxone per drug instruction daily. And from day “10” onwards until study termination, the group was administrated IP with ACC suspension twice per day.

The Group's treatment allocation are detailed in Table 16.

TABLE 16
Study groups and treatments of mice with EAE.

| Treatment | Day “0” | Dose (ml) | Treatment duration schedule |
|---|---|---|---|
| Saline | Model | 200 μl IP | 2X per day |
| | induction | | Daily |
| | | | from day “10” onwards |
| Copaxone | | Per instruction | Per instruction |
| | | | (Once a day. |
| | | | From day “0”-to day “8”) |
| ACC | | 200 μl | 2X per day |
| | | | Daily |
| | | | from day “10” onwards |
| ACC + | | 200 μl + | 2X per day |
| Copaxone | | Per instruction | Daily |
| | | | from day “10” onwards + |
| | | | Per instruction |
| | | | (Once a day. |
| | | | From day “0”-to day “8”) |

The study was done with C57BL mice that were 8 weeks old. Each study group consisted of 10 mice (n=40). Study duration was 55 days. Clinical sign scoring was recorded throughout the experiment. The scoring was evaluated as follows: (1) tail paralysis, (2) back limbs paralysis, (3) front limbs paralysis, (4) total body paralysis, and (5) mortality.

### Results

Copaxone (Glatiramer acetate) is an immunomodulator drug approved by the FDA for reducing the frequency of relapses, but not for reducing the progression of disability.

According to the results of the clinical scores that were measured throughout the duration of the experiment, presented in FIG. 8. The inventors observed that at the initial phase of the progression of the paralysis, mice treated with Copaxone and mice treated with Copaxone and ACC had a slower paralysis progression compared to mice given saline or ACC. The paralysis signs for Copaxone and Copaxone combined with ACC starting on day 11 of treatment, and of the mice treated with Saline and ACC paralysis signs started on day 10. As paralysis symptoms progressed these differences between the treatments (Copaxone, Copaxone & ACC vs. ACC and saline) were maintained until day 15. These differences are statistically significant (p<0.05).

After days 15 and 16 of treatments the symptoms have reached a certain plateau, where the Copaxone and ACC treated mice showed lower score of the paralysis signs (about 1.3) compared to the other treatments (also, statistically different). However, it is important to notice that the ACC-treated group had also lower score of the paralysis compared to the Copaxone-treated mice and saline-treated mice, which showed the same paralysis score. This effect of the Copaxone is associated with its therapeutic indication, i.e., effecting the frequency of relapse by increasing the time between their occurrences but not the progression and paralysis level of the disease.

Two distinct groups of the treated mice could be identified at the end of the study: one group of mice treated with a combination of ACC and Copaxone or just ACC, which showed lower scores of the paralysis signs and a second group of mice treated with Copaxone or saline showing a higher score of paralysis symptoms (this difference was also statistically significant).

Overall, this study indicates that ACC as a standalone treatment has an effect on reducing the severity of the paralysis symptoms compared to Copaxone or saline alone, however, the combination of ACC and Copaxone had not only a significant effect on reducing the severity of the symptoms, but also increased the period until symptoms emerged as well as slowed the progression of the symptoms. Therefore, the inventors concluded that ACC and Copaxone together had a combined effect on the paralysis symptoms of MS.

These results together with the results observed in FIG. 8 demonstrate that ACC has an anti-inflammatory effect, most likely because of neutralizing acidosis caused by the inflammation. Moreover, the effect that ACC had a synergetic effect, when combined with Copaxone strengthen the hypothesis that ACC neutralizes acidosis since one of the local acidosis effects is the inability of the immune system to work efficiently in the acidic environments.

### Example 7

### The Effect of ACC on Various Cathepsin Activities in Models with Induced Diseases that Involve High Cathepsin Activities

### 7.1 the Effect of ACC on Cathepsin B and Cathepsin S Activities in a Subcutaneous Model of Lewis Lung Carcinoma in Mice

The purpose of this study was to examine the basicity effect of ACC's basicity on Lung Lewis carcinoma tumor growth following subcutaneous injection and compare it to cisplatin (chemotherapy) treatment. The tumor growth progression was examined for a period of 12 days starting from beginning of administration.

The study was performed with C57BL/6 mice at the age of 6-8 weeks. Eight mice were allocated into each study group. Light anesthesia was administered by isoflurane inhalation. LLC cells in a concentration of 2.5×10⁵ cells in 100 μl PBS were intradermally injected (subcutaneously) into the right flank of each mice. The study consisted of the following groups: vehicle (Saline, as negative control), chemotherapy (Cisplatin, as positive control) and ACC suspension containing 0.5% (wt/v) of elemental calcium. ACC and saline were injected IP, twice a day, 7 days a week for a duration of 12 days. Cisplatin was given according to manufacturer instructions.

Ten days following tumor inoculation, when the tumor reached a volume of about >60 mm³, mice were randomly allocated into the study groups. Various treatments were administered to each group starting Day 10 onwards. The treatment duration lasted for 12 days.

Both Cathepsin B and S activities were measured using samples from the same groups according to kit instructions. It should be noted that Cathepsin S is one of the very few Cathepsin that is actually inhibited in acidic conditions and activated in alkaline ones. Cathepsin B Activity Assay Kit (Fluorometric) (ab65300) and Cathepsin S Activity Assay Kit (Fluorometric) (ab65303) by Abcam (Cambridge, UK) were used.

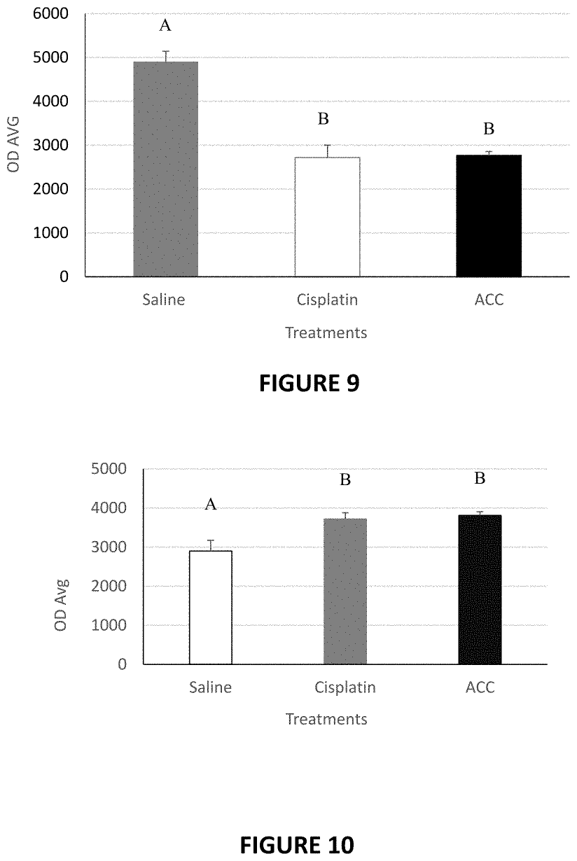

FIG. 9 shows the Cathepsin B activities and FIG. 10 shows the Cathepsin S activities in tumors of mice receiving different treatments (ACC, Cisplatin or saline). The results are described as mean±SEM, the different letters represent statistical significance (p<0.05). FIG. 9 shows that the activity of Cathepsin B was drastically decreased in tumors of mice treated with the basic ACC or cisplatin in comparison to untreated group (reduction of 43% in activity by both treatments of ACC and cisplatin). In contrast, the activity of Cathepsin S was increased in these groups as illustrated in FIG. 10 by 30% and 33 in the presence of cisplatin and ACC, respectively. This second observation is a strong indication that Cathepsin activity due to the basic characteristics of ACC is enhanced or reduced per expectations, i.e., reduction in the case of Cathepsin B but increase in the case of Cathepsin S.

### 7.2 the Effect of ACC on Cathepsin B Activity in Multiple Sclerosis (MS) Model in Mice

Multiple sclerosis MS is associated with inflammatory response, which involves local acidosis conditions. Cathepsin B activity involved in damaging the involved tissues is enhanced in such acidic pH levels. ACC can release carbonate at the mild pH levels associated with local acidosis.

This study evaluated the efficacy of ACC on MS induced in mice. This model inhibits the Th1 cytokine response, which is in the base of the inflammatory response. The mice Experimental Autoimmune Encephalomyelitis (EAE) model is an accepted model for human Multiple Sclerosis (MS). In the course of MS, an autoimmune response damages the myelin sheath. This damage disrupts the ability of parts of the nervous system to communicate, resulting in a wide range of signs and symptoms including changes in sensation, muscle weakness, movement and balance difficulties, speech and swallowing impairment, vision loss, fatigue etc. The inflammatory response generates local acidosis.

The study was performed with C57BL/6 mice at the age of 6-8 weeks. Each group consisted of 10 mice.

The myelin oligodendrocyte glycoprotein (MOG)-induced EAE model was applied in this study. MOG 35-55 solution was freshly prepared prior to each inoculation session by dissolving the RP-HPLC-purified lyophilized powder in PBS to achieve a solution at a final injected concentration of 2 mg/ml, mixed 1:1 with Complete Freund's Adjuvant (CFA) suspension, containing killed Mycobacterium Tuberculosis H37 R (at a concentration of 4 mg/ml). An aliquot of 200 microliter was injected subcutaneously into the flank of the mice.

The study consisted of two mice groups. One was a negative control, IP administered with vehicle only (Saline) twice per day from day “10” onwards until the study termination. The second was IP administered with ACC (0.5% w/v Ca) suspension, twice daily, from day “10” onwards until the study termination. The study was terminated at day 55.

Spinal cords (SC) were abstracted from 5 mice per group that were administered with saline and ACC 0.5% (w/v) calcium. Similar samples (20 mg) were taken from the SC and lysed. Each tested group contained 5 sections. Lysates were treated per instructions of Cathepsin B Activity Assay Kit (Fluorometricab65300 by Abcam, Cambridge, UK); incubation with Cathepsin B substrate for 1 hour at 37° C. to identify the activity of the lysosomal protein. The florescent reading was performed in a plate reader model Infinite 200Pro.

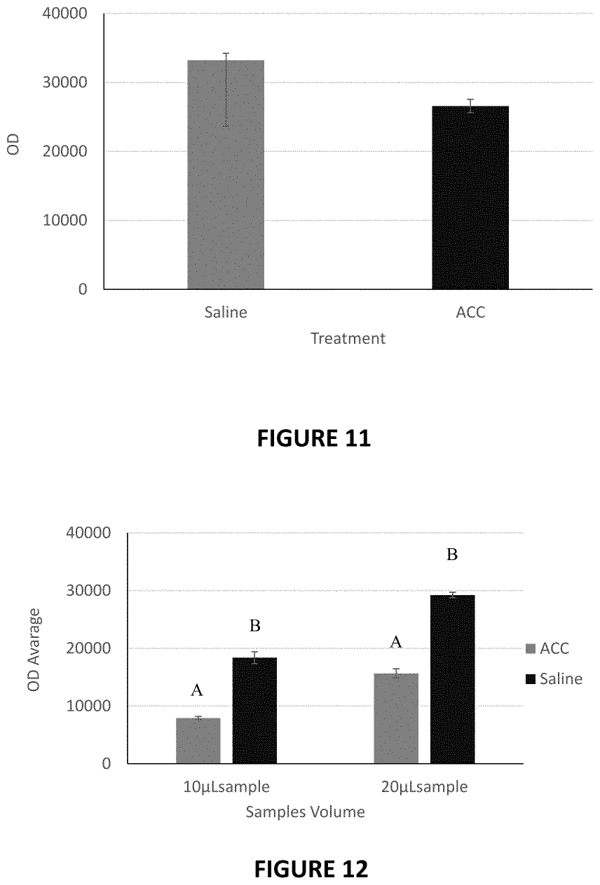

A reduction of 21% in Cathepsin B activity was measured for mice treated with ACC compared to those treated with saline is noticeable as illustrated in FIG. 11. Although the differences are not statistically significant due to the small number of tested mice per group, there is still a noticeable effect, associated with the base activity of the ACC.

### 7.3 Effects of ACC on Cathepsins B and K Activities in Collagen Induced Rheumatoid Arthritis Model in Rats

Cathepsin B and K activities are involved in inflammatory conditions associated with rheumatoid arthritis. Both Cathepsin are activated at pH levels associated with local acidosis at the joints.

This experiment evaluated Cathepsins B and K activity in rats that were induced with collagen-induced arthritis (CIA), a common model for rheumatoid arthritis (RA), which were either treated with 0.5% (w/v) elemental calcium in the form of ACC or received saline solution only.

The study was performed with 2 LEW/SsNHsd female rats at the age of 8 weeks that were immunized with a type II bovine collagen in Incomplete Freund's adjuvant (IFA), and received a boost of type II bovine collagen in Incomplete Freund's Adjuvant (IFA) 7-10 days after the first injection. The study duration was 35 days, and day 0 is the first day after immunization. The progression of the disease was recorded during the study.

One group received intraperitoneal (IP) injections of 0.2 ml with 0.5% (w/v) elemental calcium in form of ACC and the negative control was injections of 0.2 ml saline, twice a day. Also, there was a group, which received Dexamethasone (0.5 mg/Kg) orally once a day. After study termination the tissue from inflamed joints was taken from the right hind limb of each rat and the tissue was processes and evaluated for Cathepsins B (on rats treated with ACC and saline) and K activity (on rats treated with ACC, Saline and Dexamethasone) according to the kit protocols [Cathepsin B Activity Assay Kit (Fluorometric, ab65300) and Cathepsin K Activity Assay Kit (Fluorometric, ab65303)].

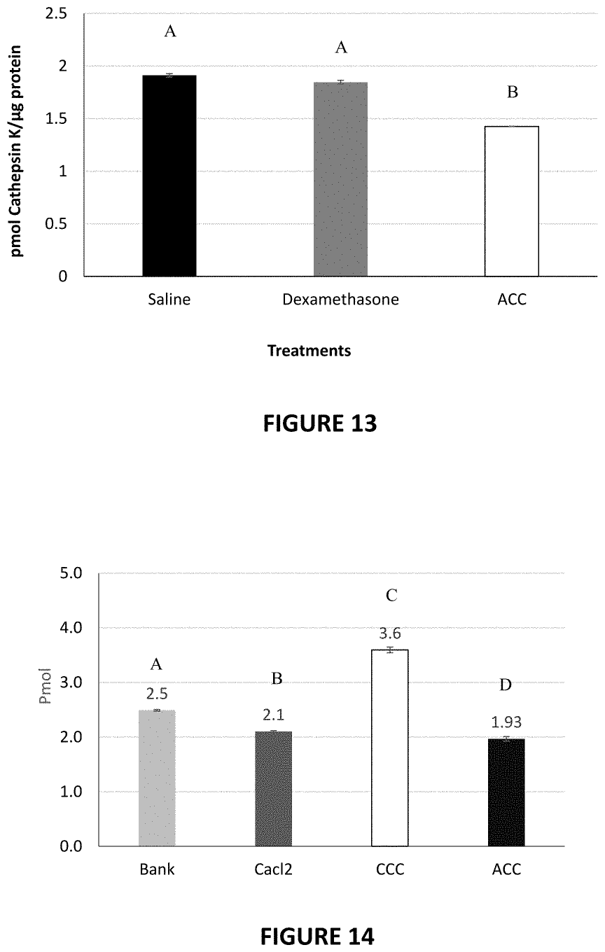

In this study the Cathepsin B activity in mice administered with ACC was reduced in the range of 50% of its activity, compared to the rats that were only administered with saline (FIG. 12). The level of Cathepsin K was reduced in a significant manner compared to rats treated with dexamethasone or saline (a reduction of 24% and 22, respectively as shown in FIG. 13). Thus, the ACC treatment has demonstrated an anti-inflammatory efficacy on the inflamed joints of the rats, even when compared to conventional treatment.

### 7.4 Effects of ACC on Cathepsin B Activity in LNCaP Clone FGC Carcinoma Prostate Cancer Cells

The purpose of this study was to examine the effect of ACC on Cathepsin B activity in LNCaP clone FGC carcinoma prostate cancer cells, following 4 passages in medium supplemented with different treatments. Cathepsin B activity was measured in the lysate of the cells and normalized after protein measurement.

The study was performed on LNCaP clone FGC carcinoma prostate cancer cells which were grown as described in previous example. In this case also suspensions of CaCl₂ and CCC at the same calcium concentrations were administered as comparative groups.

Cathepsin B activity of the ACC treated group was reduced by 30% compared to the untreated control group (see FIG. 14). The activity in the ACC treated group was lower than all other groups, although calcium chloride solutions led to lower Cathepsin B activity (by 16% compared the control). The differences between the ACC effect and all other groups were sufficient to be statistically significant (p<0.05). It is important to notice that the presence of CCC increased the Cathepsin B activity by 44% compared to control. This significant increase is interpreted because of the combined deficiency of calcium and normal biogenic pH.

### 7.5 Effects of Various Calcium Sources on Cathepsin B Activity in Lung Lewis Carcinoma (LLC) Cancer Cells

The purpose of this study was to examine the effect of ACC on Lung Lewis carcinoma (LLC) cancer cells, which were grown as described in material and methods above.

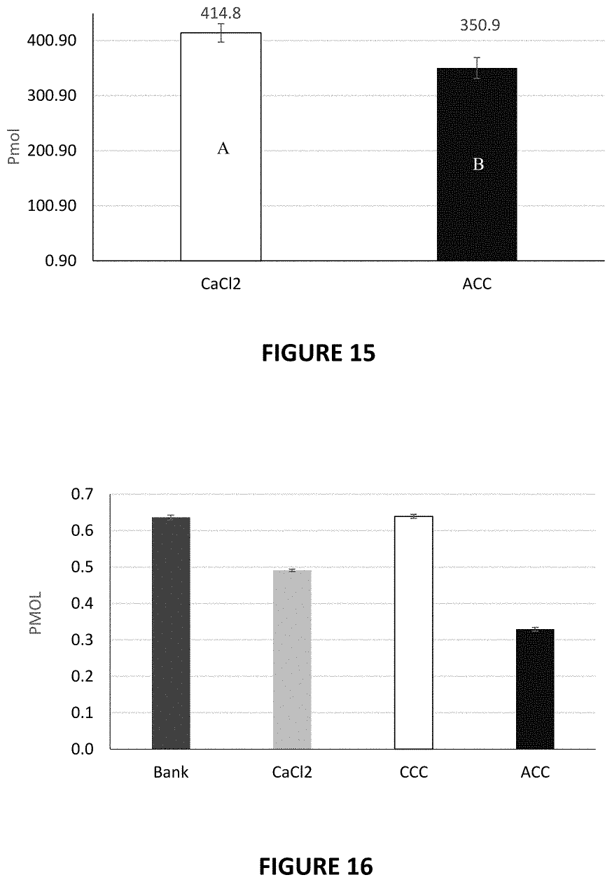

The results are illustrated in FIG. 15. Cathepsin B activity was lower by 15.4% in the ACC treated group compared to the CaCl₂ treated group. This difference was statistically significant (p<0.05) according to student t-test.

### 7.6 the Effect of ACC on Cathepsin B Activity in NIH/3T3 Normal Fibroblast Cells, in Comparison to CaCl₂, CCC and No Treatment

The purpose of this study was to examine the effect of ACC on Cathepsin B activity in NIH/3T3 normal fibroblast cells, following 4 passages in medium supplemented with the different treatments. Cathepsin B activity was measured in the lysate of the cells, normalized by the protein measurement.

The study was performed on NIH/3T3 normal fibroblast cells, which were grown as described in previous materials and method sections above. As can be seen in FIG. 16, cells treated with ACC showed the lowest activity, 49% lower compared to control and 3% compared to CaCl₂ groups. In contrast, the presence of CCC did not change the activity compared to the control. The example indicates as in previous example that Ca ions (from CaCl₂) are also effective in reducing the activity of Cathepsin B. Nevertheless, the calcium ion activity of the calcium from the chloride solution is lower in-spite of being completely soluble.

### Example 8

### A Prostate Cancer Patient Receiving ACC Treatment

A series of examples demonstrating the efficacy of ACC treatments given to patients having various diseases and conditions, known to be aggravated by local or systemic acidosis, are given in Examples 8 to 14. The ACC administration aimed at reversing these acidosis conditions and consequently assist in combating and healing the diagnosed diseases.

Example 8 reports the efficacy of ACC treatment on a hospice-stage, prostate cancer patient participating in a clinical study, an exploratory, open label study to improve the function and welfare of late-stage solid cancer subjects (with or without lung involvement) via ACC treatment, administered sublingually, concomitantly with inhalation of ACC (1%, 8 ml twice a day).

The patient, a 79-year-old male was in a late-stage prostate cancer that has exhausted all available treatments and a life prognosis of 2 to 4 months. A CT scan, done prior to the study was not aided by using a contrast agent due to renal failure. The scan showed a cyst (2×2.5×2.7 cm) in the right kidney and the other kidney had a nephroureterostomy tube. Furthermore, the scan showed two new lesions in the L4 vertebrate of the spine that were suspected as a recent metastasis.

The patient started ACC as the only treatment. He received ACC in a powder form sublingually and by inhalation of ACC suspension. The treatment started on Dec. 21, 2017, at a dose of 800 mg/day calcium in ACC, which every week was increased in 200 mg calcium in ACC up to 1800 mg/day calcium in ACC by week 5 (first week considered as weak 0). This dose was continued throughout the study up to week 11 (a total of 12 weeks study duration). The inhalation treatment started from the beginning of the treatment (week 0) and stayed constant throughout out the trial duration, twice a day 8 ml of 1% ACC suspension (0.3% calcium). During the trial duration the patient reported improvement. He continued taking ACC with the same drug regimen. The next PET-CT scan was done on Sep. 29, 2018. This scan was done using FDG as the contrast agent, and no lesions were detected. The right kidney was in a normal size and the bones showed no metastasis.

### Example 9

### A Colorectal Cancer Patient Receiving ACC Treatment

This example reports about the treatment of a patient that has exhausted all available treatments and had a prognosis of 2 to 4 months, as a part of a clinical study, an exploratory, open label study to improve the function and welfare of late-stage solid-cancer subjects (with or without lung involvement) by administering ACC sublingually and concomitantly by inhalation of ACC suspension (1%, 8 ml twice a day).

The patient, a female age 74 with advanced colorectal cancer that has metastasized to lungs, entered the study with a low oxygen saturation level (82%) and was staying in a hospice setting, connected to oxygen supply. She started ACC treatment (sublingual and inhalation) with no other conventional treatment, administered, in parallel. The treatment started on Aug. 23ed, 2018, at a dose of 1200 mg/day calcium in ACC, which every week was increased in 200 mg calcium in ACC up to 1800 mg/day calcium in ACC by week 3 (first week considered as week 0). This dose was continued throughout the study up to week 24. The inhalation treatment started from the beginning of the treatment (week 0) and stayed constant throughout out the trial duration, twice a day 8 ml of 1% ACC suspension (0.3% calcium). During the trial duration the patient reported improvement. He continued taking ACC with the same drug regimen. Shortly after starting treatment, her pain levels went down and her oxygen saturation increase. Once she resumed normal blood oxygen levels, she was released from the hospice and resumed regular day-to-day functions and activities including sport and dancing. Blood tests performed during this period revealed that the Carcinoembryonic Antigen (CEA) marker has stopped elevating and reached a plateau instead of continuous elevation as expected for a patient at this advanced stage. The CEA is a known marker for colorectal cancer, it is used to evaluate the patient state and to evaluate the efficacy of the treatment.

### Example 10

### A Non-Small Cell Lung (NSCLC) Cancer Patient Receiving ACC Treatment

A 59-year-old female patient was diagnosed with a Stage-4 NSCLC after suffering of a persisting cough. Shortly after, she began experiencing pain, low energy levels and basically stopped functioning. During this period, she started taking ACC sublingually as dry powder and by inhalations. The sublingual powder was taken at a daily dose of 2000 mg calcium in ACC (˜6670 mg of stabilized ACC), divided into 4 times. The inhalation dose was 1.14% ACC (0.45% calcium) in 10 ml suspension. It was taken 3 times a day. Several days after starting the ACC treatment she reported an improvement in coughing and in her pain levels. A biopsy was taken from the tumor to identify genetic mutations in consideration of starting a parallel immunotherapy treatment. About 6 weeks after being diagnosed and 4 weeks of ACC only administration she began receiving also Tagrisso (Osimertinib, is a monoclonal antibody, designated to treat non-small-cell lung carcinomas with a specific mutation). She continued taking both treatments, concomitantly. Her first CT scan showed a left upper lobe mass. About 20 days later the patient had a brain MRI and three brain tumors were found. About 5 months later the patient underwent a PET-CT skull base to thigh scan. The findings revealed that her upper lobe tumor has decreased in size and intensity compared to the pervious scan. The metastasis in the lymph nodes were resolved and no osseous activity (which was seen previously) has been observed.

Soon after, this patient had another brain MRI. According to the MRI findings the patient right lobe tumor disappeared (this was the biggest lesion of 1 mm), the other 2 tumors in the left lobe were not seen anymore and only stable postsurgical changes that had been seen before were detected (the patient had a left frontal temporal craniotomy prior to being diagnosed with NSCLC).

The patient showed a remarkable health improvement and a remission of the disease. According to the patient, her health provider commented that she had not seen such an improvement in such a short period of time (5 months). An additional MRI imaging of the brain taken 4 months later has confirmed that the three brain metastases have been resolved and no new brain metastasis was identified.

### Example 11

### A HER Positive Breast Cancer Patient Receiving ACC

A female patient age 39, felt a lump in her right breast. She was referred to imaging, which revealed a single lump in the right breast about 1.5 cm and suspicious lymph nodes in her armpit. Biopsy showed a malignant tumor in the breast and lymph nodes with the following characteristics: negative receptors, ki67-60% and HER2+3. A PET-CT showed breast infiltrating duct carcinoma along with metastasis in lymph nodes in the armpit, including an involvement in the Subclavicular and below the diaphragm and one metastasis in the liver. Shortly after diagnosis, she started taking ACC sublingually at a dose of 1800 mg calcium (approximately 5600 mg of ACC) per day divided into 8 portions of 200 mg calcium throughout the day. Within a month she started chemotherapy and biological treatments of Paciltaxel (chemo)-Trastuzumab (aka Herceptin, an antibody against HER2 receptor)-Pertuzumab (also an antibody that targets the HER2 receptor). The next PET-CT was performed 2 months later showed no active sign of disease, and another one was done several months after the beginning of treatment confirming the ongoing remission.

### Example 12

### ACC Effect on a Hip Stress Fracture and Consequent Inflammation of the Adductor Magnus of a Professional Marathon Runner

This example associates the use of ACC and the reduction of pains associated with stress fracture inflammation.

A top Israeli professional marathon runner, age 32, had suffered from a hip stress fracture and inflammation of the adductor magnus (a triangular muscle in the inner side of the thigh). These conditions had caused him severe pains, which had limited his ability to train and even caused him to cancel participating in sports championships. He started taking ACC powder sublingually at a dose of 1500 mg calcium a day (approximately 4700 mg ACC; 6×250 mg Ca throughout the day). About 14 days after starting the ACC treatment his pains were greatly reduced and after one more week (3 weeks after starting the treatment) his pains were completely stopped.

### Example 13

### ACC Effect on Bursitis in the Ischial Tuberosity of a Professional 3000 Meter Runner

This is another example that connects the healing of a persisting inflammation and associated pain reduction.

An Israeli 3,000 m professional runner, age 19, has been diagnosed with bursitis in both sides of the ischial tuberosity after complaining of pains and having a sensation of pulsating in this area. He was recommended by his athletic health provider to receive local steroid injections into the bursitis near the ischial at both sides. He declined the steroid treatment and started taking ACC at a dose of 1,600 mg calcium daily (8×200 mg Ca throughout the day). About 45 days after starting the treatment his pulsating feeling and pains were almost completely faded (95% less pain). Since then, he resumed his regular training and competition program.

### Example 14

### ACC Effect on Inflammation and Swelling of the Knees of a Heavy Weightlifter and Cross-Fit Athlete

Another example is given for sport related pains due to a chronic inflammation that were completely faded after ACC administration.

A female athlete, age 29 is a top cross-fit professional and regularly practices heavy weightlifting. She was suffering from local swelling, redness and pains in the knees and the area felt warm for years. The chronic inflammation has limited her ability to train as well as fulfilling her daily activities. She has tried numerous treatments including resting, physiotherapy and non-steroid anti-inflammatory medications (Etopan), which did not help her, especially when she wanted to increase the strain on the knees during training. She started taking ACC at a daily dose of 800 mg calcium (200 mg×4 times a day). A few days after starting ACC treatment her swelling, redness and local warmness were reduced as well as the pain in her knees.

### Example 15

### Evidence for ACC Fraction Passing Through the Stomach without Decomposition of Carbonate

The following experiments were performed to demonstrate that if 2 or 3 ACC tablets are administrated together, they can completely quench the acidity of anticipated gastric acid volumes (at a certain given time). This residual fraction can reach the intestine tract, release its nanometric ACC load and transfuse through the intestine's mucous membrane. A volume of 100 ml of two different gastric acid concentrations was used in the experiments, based on various reports about the maximum volumes and concentrations found during a given time inside the stomach.

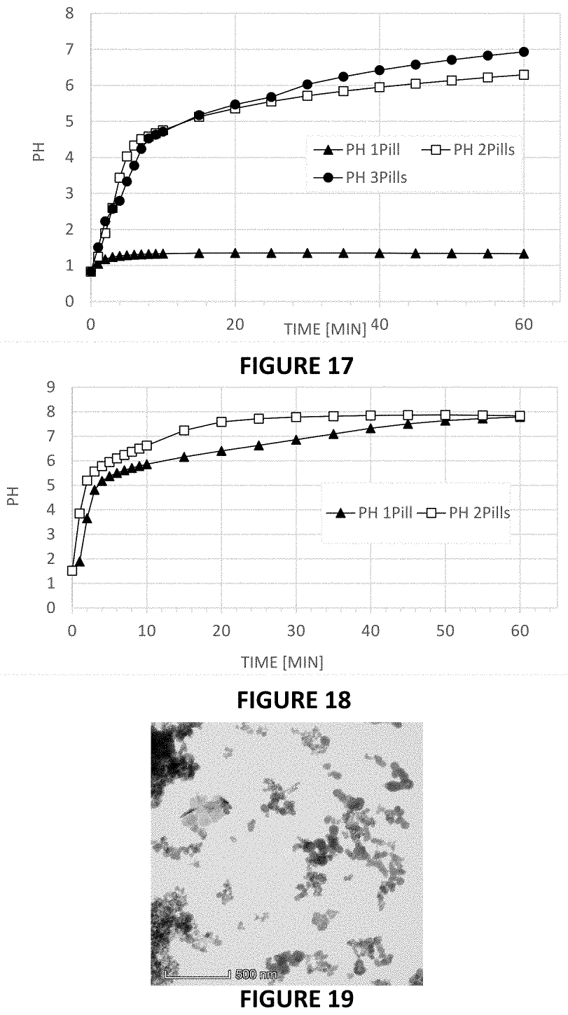

A solution of 100 ml 0.16 N HCl (pH 0.83), was placed in a cup, topped with a cover having a hole through which a pH electrode probe was inserted. The cup was placed over a magnetic stirrer, with a stirring bar inside. Then, ACC (Density®) tablet(s) were added while the pH was constantly recorded was monitored over a period of 1 hour at room temperature (25° C.). Each tablet consists of 200 mg of calcium and the calculated amount of ACC in each tablet is 670 mg. As exhibited in FIG. 17, three consecutives experiments were performed, the first one with the addition of 1 ACC tablet, the second with 2 ACC tablets and the third with 3 ACC tablets. All superimposed graphs display a raise of the pH until a plateau is reached after 10 to 15 minutes. One tablet is not enough to significantly raise the pH beyond 1.34 but it should be remembered that this is change means the quenching of 70% of the solution acidity. In contrast, the pH attained a constant value of pH=6.3 with the addition of two tablets and reached a constant value of pH=6.9 with the addition of three tablets as observed in FIG. 17. The main raise of the pH occurred during the first 10 minutes after the addition of the tablet(s) to the acidic solution.

A second set of experiments was performed at higher pH, which is also reported as a typical volume and pH found in the stomach in a given time. A volume of 100 ml solution of 0.032 N HCl (pH 1.5) was used as in the first set of experiments. As shown in FIG. 18, two consecutives experiments were performed. In the first one, a single tablet was added and the second consisted of adding 2 tablets. Both superimposed graphs exhibited a sharp raise of the pH to about 6.5 and 7.5, respectively. A plateau is reached after 40 minutes, when the pH reaches a constant value of pH 7.83 for the addition of 1 or 2 tablets. The major raise of the pH occurred during a period of the first 10 minutes starting from the addition of tablet(s) to the acidic solution.

The experiments indicate that taking a relatively low dose of 400 or 600 mg of Ca in the form of ACC in a single administration is sufficient to quench the stomach acidity and allow a fraction of the ACC particulates enter the intestine tract without decomposition of the nanoparticles or the released carbonate anions.

### Example 16

### Various Batches of Enteric Capsules Filled with ACC of Different Formulations

Enteric encapsulation of ACC is another means to administer ACC without its dissolution and partial decomposition to CO₂ in the gastro tract, before reaching the intestine and enter the body in the form of ACC primary nanoparticles. In this case, the enteric shell degrades only at the close to neutral pH in the intestine. The following examples demonstrate the capability of forming and utilizing efficiently the concept of enteric administration.

Materials used for enteric encapsulation included CAP (Cellulose Acetate Phthalate), CAT (Cellulose Acetate Trimellitate), PVAP (Polyvinyl Acetate Phthalate), and HPMCP (Hypromellose Phthalate).

### Introduction

ACC's morphology is constructed from primary nanoparticles aggregated into high-surface-area clustered particles. ACC manufactured by Amorphical has a specific surface area in the range of 30 to 60 m²g⁻¹. Electron microscopy and nitrogen sorption analysis revealed that the primary particles are in the range of 10 to 100 nm and the surface area fits particles in the average range of 50 nm in diameter. (see FIG. 19).

The aim of the study is to develop a target enteric capsule filled with as much ACC as possible but simultaneously, allow the powder to disperse back into the original particle sizes used in the process, hence allowing adequate disintegration/dispersion of the particles in the upper GI tract's fluids. There is a need to formulate ACC powder in such a manner that ensures a maximal dispersion over hard aggregation/agglomeration of the ACC powder in water medium.

Due to the natural tendency of ACC to form large aggregate/agglomerate, which by themselves are too large for good dispersion, it is preferred to first mill the as-synthesized aggregates/agglomerates into smaller clusters in the range below 10 micrometers. This process further increases the volume uptake of the ACC powder.

However, for increasing the content of the capsule, it is preferable to compact the particles as much as possible. Nevertheless, this process can be counterproductive by generating hard agglomerates that are not easily dispersed back in the GI's fluids. Therefore, processing techniques that overcome the tradeoff between high dosage and the required good dispersion, are needed.

### Materials and Methods

Five different types for enteric capsules (Vcaps® Enteric Capsugel; “cellulosic enteric derivatives”, Lonza, Basel, Switzerland) were filled with ACC in different powder filling/additives//treatment processes (see Table 17). In order to find, which of them ensures the best ratio between maximum ACC powder loading and ACC dispersion in water medium, imitating the upper intestinal fluids and their physical motion.

The major productions steps consisted of mixing the ingredients, manually or by using Diosna mixer (Diosna, Germany), followed by a roller compactor granulator (HM GA-DH120 Dry granulator, HM Pharmachine, China) with different sieve such as 1 mm or 1.4 mm. Another option was using an Oscillating granulator (Rotorgran MK IV, Manesty Machines Ltd., Liverpool United Kingdome) for size reduction with, for example, a 50-mesh sieve (having a pore size of ˜300 μm) or an 80-mesh sieve (having a pore size of ˜177 μm). The compounds were mixed again. Then the product was ready to be inserted into an enteric coating which in this case was Vcaps® enteric capsugel (Lonza, Basel, Switzerland). The production steps for each batch are summarized in Table 17. The different amounts of ACC inserted into each Vcap were between 271 mg to 771 mg, as presented in Table 17 for each batch.

### Dispersion Test (Method, Sample Size, Interval Times, Analysis)

The dispersion test method described by USP monograph No. <711> for calcium carbonate tablet dissolution test method was modified specifically for this study. The following ACC dispersion parameters are specified in U.S. Pharmacopeia (USP) official monograph for calcium carbonate tablet dissolution and were used for the dispersion of enteric ACC-capsules in Deionized water (at pH 7.0).

At various intervals, 10 ml samples were drawn from the top of the stirred suspension. The dispersion can be visually realized by its milky appearance and its homogeneity.

The dispersion measurement was performed according to the following procedure: (a) The dispersion vessel was filled up with Deionized water (pH 7) up to 900 ml; (b) The temperature was set to 37.5° C.; (c) The temperature of the dispersion medium was maintained at 37.5° C.; (d) One filled Enteric Vcap ACC-Capsule was added to each vessel; (e) The operation was started at stirring speed of 75 rpm; (f) After about 37 minutes the Vcap disintegrate in water and ACC released from the capsule. Aliquots of 10 mL samples of the dispersion ACC in water medium were sampled from the suspension top of each vessel for analysis without filtration; (g) The 10 mL samples of the dispersion medium were weighed into a 250-mL beaker; (h) Amounts of 10 ml HCl 3M, 90 mL deionized water and 15 mL sodium hydroxide 1N solution were added; (i) An amount of 300 mg of Hydroxy Naphthol Blue indicator was added and mixed; and (j) The calcium ions in the solution were titrated with EDTA 0.005M until a stable blue color is obtained.

The percentage of the calcium carbonate quantity was calculated based on the below equation:

Result=[(VS−VB)×M×F×100]/W; VS=Volume of the Titrant consumed by the Sample solution (ml); VB=Volume of the Titrant consumed by the Blank (ml); M=Titrant molarity (mmol/ml); F=Equivalency factor for CaCO₃, 100.09 mg/mmol; W=Weight of calcium carbonate taken (mg).

### Powder Characterization

As indicated in Table 17 the procedure methods, in the first three batches the powdered ACC was subjected to a Roller Compactor. This process was done to reduce the volume to weight ratio (bulk density) of the flowing ACC powder, thus allowing to maximize the amount of ACC in the Enteric Capsule. It resulted with a very dense ACC that did not disperse in a satisfactory way. With the two last batches (Batch 4 and Batch 5) the ACC-Powdered was grinded in an Oscillating Granulator with 80 mesh sieves. This procedure resulted with lower loads of ACC amounts within the enteric capsules but with the most adequate dispersion. The dispersion inside the GI's fluids relies on process engineering and formulations of the ACC powders. Some critical elements include but not limited to the following segments:
Nanoparticle structure, via measurements of surface area (BET), porosity, density.
Agglomerate/aggregate “hardness” and size, especially after milling and sieving.
Compaction volume/dose capability of the powders as a function of applied pressure, without significantly affecting the disintegration, dispersion, or dissolution of the compacted powders.
The type and amounts of additives that allow higher compaction capabilities with adequate disintegration, dispersion, or dissolution of the compacted powders.

The results of the dispersion tests (for each time period), bulk density and BET for each of the five batches are presented in Table 17.

### Results

Table 17 below summarizes the components of each batch, procedure methods, ACC amount in each Vcap, and the results of the dispersion tests, bulk density, and BET.

TABLE 17
Different compositions of ACC for enteric capsules and production
methods, amounts, dispersion, bulk density and BET results

| | Batch Number | | | | |
|---|---|---|---|---|---|
| | 1 | 2 | 3 | 4 | 5 |
| % ACC (API) | 98 | 97.5 | 93.5 | 100 | 99 |
| % CCS | 1 | 1 | 5 | 0 | 0 |
| (superdisintegrant) | | | | | |
| % Aerosil 200 | 1 | 1 | 1 | 0 | 1 |
| (Anticaking Agent | | | | | |
| and Glidant) | | | | | |
| % Magnesium | 0 | 0.5 | 0.5 | 0 | 0 |
| Stearate | | | | | |
| (Lubricant) | | | | | |
| Process Methods | Mixing ACC + | Mixing ACC + | Size reduction | Size reduction | Size reduction |
| | Aerosil + CCS | Aerosil + CCS | to ACC in | to ACC in | to ACC in |
| | in Diosna mixer | in Diosna mixer | Oscillating | Oscillating | Oscillating |
| | (Diosna, Germany) | followed by | granulator with | granulator with | granulator with |
| | followed by | the Addition of | 50 mesh sieves | 80 mesh sieves | 80 mesh sieves |
| | Roller Compactor | Mg Stearate and | followed with | | |
| | (HMGA-DH120 Dry | mixing without a | mixing ACC from | | |
| | granulator, | Chopper in | Oscillating | | |
| | HM Pharmachine, | Diosna mixer | granulator + | | |
| | China). | followed by | Aerosil + CCS | | |
| | | Roller Compactor | in Diosna mixer, | | |
| | | | followed with | | |
| | | | the addition of | | |
| | | | Mg Stearate and | | |
| | | | mixing without | | |
| | | | Chopper in | | |
| | | | Diosna mixer. | | |
| | | | Followed with | | |
| | | | Roller Compaction | | |
| Granulator Size in | 1 mm Sieve | 1 mm Sieve | 1.4 mm Sieve | 50 mesh Sieve | 80 mesh Sieve |
| Mesh | | | | | |
| Average amount of | 601.6 | 614 | 771.1 | 323.4 | 271.2 |
| ACC-Powdered | | | | | |
| loaded in enteric | | | | | |
| capsules | | | | | |

| | Capsules Batch No. | | | | |
|---|---|---|---|---|---|
| | Average Dispersion Percentage Value of capsules filled with ACC | | | | |
| | formulated and/or processed with different protocols (Shearing Methods) | | | | |
| Time Interval (min) | 1 | 2 | 3 | 4 | 5 |
| 37 | 10 ± 1.2 | 8 ± 0.5 | 42 ± 9.9 | 86 ± 3.3 | 102 ± 2.67 |
| 60 | 16 ± 1.4 | 8 ± 0.7 | 48 ± 2.1 | 89 ± 1.8 | 101 ± 0.59 |
| 130 | 20 ± 1.1 | 20 ± 2.4 | 43 ± 1 | 91 ± 2.5 | 101 ± 0.31 |
| 180 | 25 ± 1.5 | 28 ± 2.2 | 42 ± 1.2 | 93 ± 2.8 | 102 ± 0.30 |
| Bulk Density(g/ml) | N/A | N/A | 0.66 | 0.22 | 0.21 |
| BET (m²/g) | N/A | N/A | 37.2 | 45.4 | 45.4 |

In this experiment the best results were obtained with batches 4 and 5, since they gave the highest dispersion rates of 93% and 102%, respectively. However, there is a tradeoff with the amount of ACC powder that can be loaded into the capsules.

### Example 17

### Methods and Means to Increase ACC Content in an Enteric Formulation

Hereinafter is a series of procedures and ingredients that allow to increase the ACC content within an enteric coating while maintaining high dispersion rates. A series of anticaking agents were added to the formulations of the ACC-powdered to prevent the formation of lumps in the dispersion medium. Anticaking agents prevent aggregating, agglomerating and maintain a free flow of compacted powders. Table 18 lists potential of anticaking agents to be formulated with the active divalent metal carbonate substance, compacted in capsules.

TABLE 18
A list of optional Anticaking agents

| Chemical | Molecular | CAS Registry | Functional |
|---|---|---|---|
| Name | Formula | Number | Category |
| Magnesium | MgO•SiO₂•ₓH2O | 1343-88-0 | Anticaking agent, glidant |
| Silicate | | | |
| Magnesium | Mg₂Si₃O₈•ₓH₂O | 14987-04-3 | Anticaking agent, glidant, therapeutic |
| Trisilicate | | | agent |
| Tricalcium | Ca₃(PO4)₂ | 7758-87-4 | Anticaking agent; buffering agent; |
| Phosphate | | | dietary supplement; glidant; tablet |
| | | | and capsule diluent |
| Talc | Mg₆(Si₂O₅)₄(OH)₄ | 14807-96-6 | Anticaking agent; glidant; tablet and |
| | | | capsule diluent; tablet and capsule |
| | | | lubricant |
| Hydrophobic | Aerosil R972 | 68611-44-9 | Anticaking agent; emulsion stabilizer; |
| Colloidal | | | glidant; suspending agent; thermal |
| Silica | | | stabilizer; viscosity-increasing agent |
| Magnesium | MgO | 1309-48-4 | Anticaking agent; emulsifying agent; |
| Oxyde | | | glidant; tablet and capsule diluent |
| Calcium | CaSiO₃ | 1344-95-2 | Adsorbent; anticaking agent; opacifier; |
| Silicate | | | tablet filler |

The typical percentage in w/w of such added anticaking agent is 1 to 6% to enhance the disruption of the aggregates to obtaining a fine particles dispersion whilst suppressing the formation of floating chunks in the medium. Table 19 suggest processes for preparing capsules of ACC or an alternative metal carbonate that incorporate such anticaking agents.

TABLE 19
Enteric coating formulations

| | Batch Number | | | | |
|---|---|---|---|---|---|
| | 1 | 2 | 3 | 4 | 5 |
| Batch Name | N/A | N/A | N/A | N/A | N/A |
| ACC (API) in | API Size Reduction | API Size Reduction | API Size Reduction | API Size Reduction | API Size Reduction |
| Roller Compaction\ | with Hammer Milling | with Hammer Milling | with Hammer Milling | with Hammer Milling | with Hammer Milling |
| Dry Granulation | (INNO HM 300). | (INNO HM 300). | (INNO HM 300). | (INNO HM 300). | (INNO HM 300). |
| Mode in purpose to | The Powdered API | The Powdered API | The Powdered API | The Powdered API | The Powdered API |
| yield an enteric | is run through a | is run through a | is run through a | is run through a | is run through a |
| capsule filled with | Roller Compaction | Roller Compaction | Roller Compaction | Roller Compaction | Roller Compaction |
| two to three times | followed with a | followed with a | followed with a | followed with a | followed with a |
| more of ACC | Dry Granulator in | Dry Granulator in | Dry Granulator in | Dry Granulator in | Dry Granulator in |
| powdered than | 50 mesh sieve in | 50 mesh sieve in | 50 mesh sieve in | 50 mesh sieve in | 50 mesh sieve in |
| previously | obtaining ACC | obtaining ACC | obtaining ACC | obtaining ACC | obtaining ACC |
| | powdered which is | powdered which is | powdered which is | powdered which is | powdered which is |
| | milled once more | milled once more | milled once more | milled once more | milled once more |
| | with Hammer Milling | with Hammer Milling | with Hammer Milling | with Hammer Milling | with Hammer Milling |
| | with 0.42 mm sieve. | with 0.42 mm sieve. | with 0.42 mm sieve. | with 0.42 mm sieve. | with 0.42 mm sieve. |
| Powder Vibration | Uniform Powder | Uniform Powder | Uniform Powder | Uniform Powder | Uniform Powder |
| Drum Sifter | Size Distribution | Size Distribution | Size Distribution | Size Distribution | Size Distribution |
| | 420 microns | 420 microns | 420 microns | 420 microns | 420 microns |
| listed below | Internal Addition of | Internal Addition of | Internal Addition of | Internal Addition of | Internal Addition of |
| | a Superdisintegrant | a Superdisintegrant | a Superdisintegrant | a Superdisintegrant | a Superdisintegrant |
| | added with blending | added with blending | added with blending | added with blending | added with blending |
| | to the API before | to the API before | to the API before | to the API before | to the API before |
| | proceeding through | proceeding through | proceeding through | proceeding through | proceeding through |
| | Roller Compaction\ | Roller Compaction\ | Roller Compaction\ | Roller Compaction\ | Roller Compaction\ |
| | Dry Granulation | Dry Granulation | Dry Granulation | Dry Granulation | Dry Granulation |
| | Mode, to effectively | Mode, to effectively | Mode, to effectively | Mode, to effectively | Mode, to effectively |
| | break the formation | break the formation | break the formation | break the formation | break the formation |
| | of aggregates in | of aggregates in | of aggregates in | of aggregates in | of aggregates in |
| | solution | solution | solution | solution | solution |
| % Sodium Alginate | 3 | 3 | 3 | 3 | 3 |
| % Sodium Starch | 3 | 3 | 3 | 3 | 3 |
| Glycolate | | | | | |
| % PARTECK CCS | 3 | 3 | 3 | 3 | 3 |
| Weak Acids as | Internal Addition of | Internal Addition of | Internal Addition of | Internal Addition of | Internal Addition of |
| Disintegrants | a Weak Acid | a Weak Acid | a Weak Acid | a Weak Acid | a Weak Acid |
| listed below | added with blending | added with blending | added with blending | added with blending | added with blending |
| | to the API before | to the API before | to the API before | to the API before | to the API before |
| | proceeding through | proceeding through | proceeding through | proceeding through | proceeding through |
| | Roller Compaction\ | Roller Compaction\ | Roller Compaction\ | Roller Compaction\ | Roller Compaction\ |
| | Dry Granulation | Dry Granulation | Dry Granulation | Dry Granulation | Dry Granulation |
| | Mode, to effectively | Mode, to effectively | Mode, to effectively | Mode, to effectively | Mode, to effectively |
| | break the formation | break the formation | break the formation | break the formation | break the formation |
| | of aggregates in | of aggregates in | of aggregates in | of aggregates in | of aggregates in |
| | solution | solution | solution | solution | solution |
| % Citric Acid | 0.5-2 | 0.5-2 | 0.5-2 | 0.5-2 | 0.5-2 |
| % Malic Acid | 0.5-2 | 0.5-2 | 0.5-2 | 0.5-2 | 0.5-2 |
| % Propionic Acid | 0.5-2 | 0.5-2 | 0.5-2 | 0.5-2 | 0.5-2 |
| Anticaking Agents | External Addition of | External Addition of | External Addition of | External Addition of | External Addition of |
| listed in Table 18 | an Anticaking Agent | an Anticaking Agent | an Anticaking Agent | an Anticaking Agent | an Anticaking Agent |
| added in External | added with blending | added with blending | added with blending | added with blending | added with blending |
| fashion to the | to the blend | to the blend | to the blend | to the blend | to the blend |
| Disintegrants and | obtained after | obtained after | obtained after | obtained after | obtained after |
| Superdisintegrants | Roller Compaction\ | Roller Compaction\ | Roller Compaction\ | Roller Compaction\ | Roller Compaction\ |
| Excipients | Dry Granulation | Dry Granulation | Dry Granulation | Dry Granulation | Dry Granulation |
| | Mode as defined in | Mode as defined in | Mode as defined in | Mode as defined in | Mode as defined in |
| | Procedures listed | Procedures listed | Procedures listed | Procedures listed | Procedures listed |
| | above | above | above | above | above |
| % Aerosil R972 | 5-6 | 5-6 | 5-6 | 5-6 | 5-6 |
| % Calcium Silicate | 5-6 | 5-6 | 5-6 | 5-6 | 5-6 |
| % Tricalcium | 5-6 | 5-6 | 5-6 | 5-6 | 5-6 |
| Phosphate | | | | | |
| % Talc | 5-6 | 5-6 | 5-6 | 5-6 | 5-6 |
| % MgO | 5-6 | 5-6 | 5-6 | 5-6 | 5-6 |

There are several modes for incorporating disintegrating and other agents with the active substance-powders, for example: (a) Internal Addition (Intragranular); (b) External Addition (Extragranular); and (c) Combined Internal and External.

In external addition method, the disintegrant is added to the sized dry granulation of the active substance by mixing, after the roller compaction/dry granulation of the ACC—was done. In the internal addition method, the disintegrant is mixed with other powders before the roller compaction/dry granulation. Thus, in this method the disintegrant is incorporated within the dry granules. When these methods are used, part of the disintegrant can be added internally and part externally. This partition procedure can provide immediate disruption of the aggregate and avoid the formation of lumps in the GI tract.

### Weak Acids as Disintegrants

It is feasible for instance to mix a weak acid such as Citric Acid in a solid powder form, that will react exothermically with the carbonate active substance, once exposed to a water-based body fluid. The immediate release of CO₂ by partial decomposition of carbonate ions will facilitate the dispersion of the compacted aggregates into small particles, easing the release of the active substance in the upper GI tract.

### Example 18

### Enteric Coating and Matrix of Tablets & Pellets

Enteric polymer coating can also be used to avoid drug release in the gastric fluid to bypass the stomach's acid strong and undesired interactions with the active pharmaceutical substance; then, to release the tablet or pellet content or in the GI tract at much milder pH. Different types of coating equipment can be used to process the polymers coating solutions:
Fluidized bed coater
Coating pan with spray nozzle: spray nozzle diameter 1.2 mm, spray rate of 4 to 6 g/min. atomizing pressure 1.2 bar.

The polymer coating solutions can consist of Shellac (a bioadhesive polymer), Hypromellose (Hydroxypropyl Methyl Cellulose, a semisynthetic polymer), SEPIFILM SN, PVP (polyvinylpyrrolidone), Acetylated Monoglyceride.

Another application is in which the active substance is embedded in a polymeric matrix for the formulation of modified-release oral-dosage forms and is well suitable for direct compression (DC) process. This application ensures a consistently effective treatment within the therapeutic window. The advantages of oral modified release are listed herein: therapeutic efficacy, reducing side-effects, minimizing variability, improving patient compliance, optimizing performance, prolonging action, enhancing bioavailability, and bypassing active substance hurdles.

Examples for enteric polymers are: Shellac SSB Pharma, Parteck SRP 80 (Polyvinyl alcohol). They can be used in a variety of ways to develop oral formulations with delayed and controlled release. One is the use as matrix-forming polymers in tablets and pellets. Another implication is to avoid dose dumping, a critical topic for modified release formulations. SRP 80 or SSB pharma are intended to release the drug in desired concentrations for a prolonged period. Therefore, they typically contain larger amounts of the active substance than in a single dose formulation.

The polymer powder and active substance are mixed in the dry state and compressed as they are. The compression causes the polymer particles to form a coherent matrix from which the drug is slowly released by diffusion through pores or disintegration. Depending on the desired release profile and on the polymeric matrix profile, 15% to 50% w/w of polymer powder is intended to be mixed with the active substance and additional excipients before being processed by compaction and milling. Alternatively, mixed powder formulation will be sprayed by solution or emulsion of the polymer and then quickly dried in a fluidized bed, by a spray dryer or other drying techniques.

Tablets consisting of compacted active substance and excipients can be coated with the desired composition by various conventional techniques used for coating and drying tablets. In the case of tablets, the polymer fraction can be in the range of 1 to 5 wt % of the total weight of the tablet.

The selected formulations/processes for preparing the enteric pellets and tablets will be analyzed by the dispersion test method for quantitative comparison between the different formulations/processes in a manner that predicts the greatest protection in acidic conditions and dispersion at near neutral pH. The formulation with the best dispersion value outcome will be selected for further preclinical and clinical studies.

### Example 19

### Development of Appropriate Formulations/Processing for Enteric Capsules of ACC

Initial trials were preformed to determine the feasibility of production enteric ACC capsules. The main goal was producing a formulation which has a content of not less than 200 mg elemental calcium (in the form of ACC). Since ACC has a low bulk density, a granulation process must be done to complete this task. Dry granulation was chosen due to the sensitivity to moisture of the ACC. The equipment that was used is the roll compactor, which is also used to produce the ACC granules for tablet production.

Different methods of milling before and after compactions were tested. The addition of different excipients to enhance the granules dispersion and powder flowability were tested. In addition, formulations with effective disintegrants were tested in an attempt to disperse rapidly the ACC content once the compacted formulation is released from the capsule at near neutral pH.

### Equipment

### Hammer Mill—HM-300 Manufactured by Inno-Machinery

The main parameters for controlling the hammer-mill particle size output are the sieve hole size, the main speed, and feeding speed (set by interval time)

### Roll Compactor—GA-DH120 Manufactured by HM Pharmachine

The roll compactor main parameters include the Pressure (which is set to permanent setting), auger feeder speed (faster means more compacted material) and the main roller speed (faster means less compacted material).

### Capsulating Machine—NJP1200 Manufactured by Alliance Machinery (Shanghai).

The capsule machine is fully automatic. The dosing is controlled by 5 stages of pins that compress the powder into a dosing disk. In all the tests recorded in this example the pins height setting was not changed.

### Results

The following data in Table 20 summarizes the processing of 3 different batches, each include 4 to 8 different formulations or production process.

TABLE 20
Summary table of different processing parameters

| | | | | Compaction | | | | | |
|---|---|---|---|---|---|---|---|---|---|
| | | | | Roll | | Final milling | | | |
| | ACC milling with | | | compactor | | Rotary | | | |
| | Silica-Hammer Mill | | | Feed | Roller | mill | Hammer mill | | |
| | | Sieve | Feed | motor | speed | Sieve | | Sieve | Feed |
| Batch | Speed | opening | interval | setting | setting | opening | Speed | opening | interval |
| # | [rpm] | [mm] | [sec] | [Hz] | [Hz] | [mm] | [rpm] | [mm] | [sec] |
| 1, 3, 5 | 1500 | 0.4 | 5 | 16 | 25 | — | 1500 | 0.4 | 6 |
| 2, 4, 6 | 1500 | 0.4 | 5 | 16 | 25 | 1.0 | — | — | — |
| 7-14 | 1500 | 0.2 | 6 | 20 | 25 | — | 1500 | 0.8 | 6 |

Table 21 summarizes the formulations used in this set of experiments

TABLE 21
Formulation summary

| | | AVICEL | AVICEL | Kollidon | Kollidon | DISOLCEL | | Kollidon | Kollidon | AEROSIL | Mg. |
|---|---|---|---|---|---|---|---|---|---|---|---|
| Batch | ACC | 101 | 102 | 25 | 64 | CCS | Primojel | CL | CLF | 200 | St |
| # | [%] | [%] | [%] | [%] | [%] | [%] | [%] | [%] | [%] | [%] | [%] |
| 1, 2 | 84 | — | 12 | — | — | 3 | — | — | — | 1 | — |
| 3, 4 | 75 | — | 17 | 2 | — | 5 | — | — | — | 1 | — |
| 5, 6 | 93.6 | — | — | — | — | 5 | — | — | — | 1 | 0.4 |
| 7 | 83 | 10 | — | — | — | 6 | — | — | — | 1 | — |
| 8 | 83 | 10 | — | — | — | — | 6 | — | — | 1 | — |
| 9 | 83 | 10 | — | — | — | — | — | 6 | — | 1 | — |
| 10 | 83 | 10 | — | — | — | — | — | — | 6 | 1 | — |
| 11 | 92 | 5 | — | — | 2 | — | — | — | — | 1 | — |
| 12 | 87 | 5 | — | — | 2 | 5 | — | — | — | 1 | — |
| 13 | 92 | — | — | — | 2 | 5 | — | — | — | 1 | — |
| 14 | 94 | — | — | — | — | 5 | — | — | — | 1 | — |

The materials used for the formulations are described in Table 22, including their chemical definition and their function.

TABLE 22
Materials details and function

| Product name | Chemical name | Abbreviations | Main function |
|---|---|---|---|
| ACC | Amorphous Calcium | ACC | API |
| | Carbonate | | |
| Avicel ® PH-101 | Microcrystalline | MCC | Filler/Binder |
| | cellulose | | |
| Avicel ® PH-102 | Microcrystalline | MCC | Filler/Binder |
| | cellulose | | |
| Kollidon ® 25 | Polyvinylpyrrolidone | PVP | Binder |
| Kollidon ® VA 64 | Vinylpyrrolidone-vinyl | PVP/VA | Binder |
| | acetate copolymer | Copolymer | |
| DISOLCEL ® | Croscarmellose sodium | CCS | Super-disintegrant |
| Primojel ® | Sodium Starch | SSG | Super-disintegrant |
| | Glycolate | | |
| Kollidon ® CL | Polyvinylpyrrolidone, | PVP | Super-disintegrant |
| | crosslinked | | |
| Kollidon ® CL-F | Polyvinylpyrrolidone, | PVP | Super-disintegrant |
| | crosslinked | | |
| AEROSIL ® 200 | Silicon dioxide | Silica | Flow/anti-caking |
| Mg•st | Magnesium stearate | — | Lubricant |

### Test Procedure

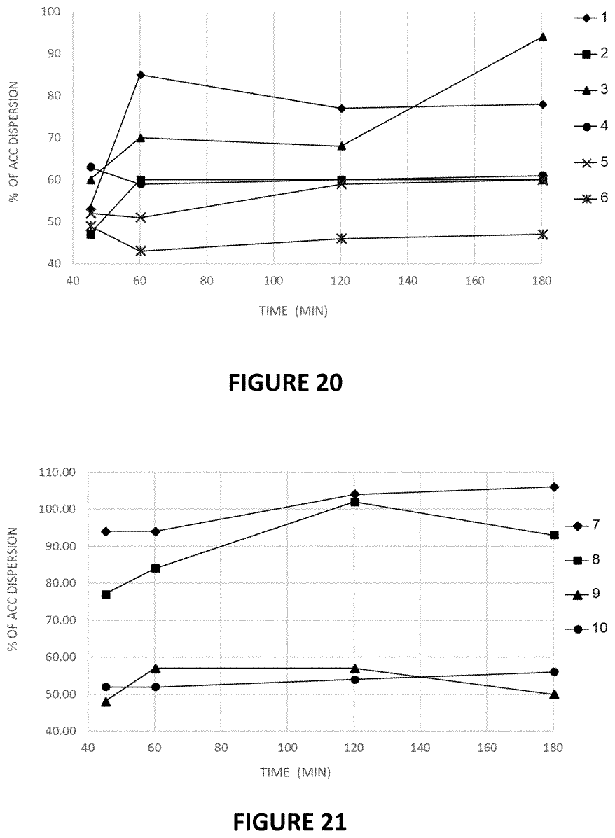

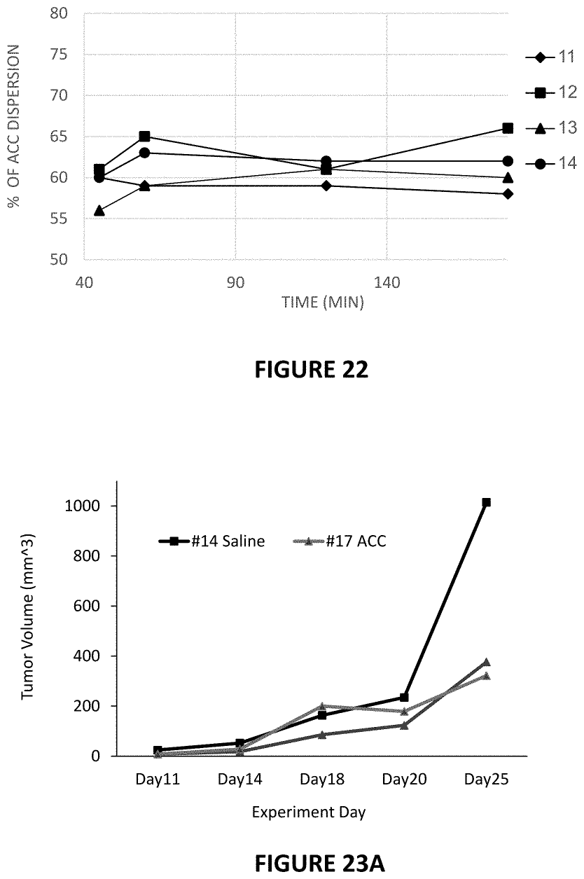

The dispersion measurement was performed according to the following procedure. The dispersion vessel was filled up with Deionized water (pH 7) up to 900 ml. The temperature was set to 37.5° C. and maintained at this temperature. One filled Enteric Vcap ACC-Capsule was added to each vessel. The operation was started at stirring speed of 75 rpm. After tens of minutes the Vcap disintegrated in water and ACC released from the capsule. 10 mL samples of the dispersed ACC in water were taken from the top of each vessel for analysis without filtration. They were weighed into a 250-mL beaker. This sample was analyzed for its calcium content by the standard titration method. The results are illustrated in FIGS. 20-22.

### Example 20

### ACC Solution Administered Intravenously (IV)

### Introduction

The following series of experiments evaluated the safety of administrating ACC solution into the blood system via intravenous (IV) infusion. This administration route is the best way to reach areas of acidosis that might be more difficult to reach by other administration routes.

### Materials and Methods

Experiment 1—0.3% Ca. In this experiment 6 Sprague Dawley (SD) female rats that were cannulated to their jugular vein were administered with 4 solutions: (1) 0.3% Ca in ACC stabilized with 10% TP or (2) 0.3% Ca in ACC stabilized with 20% CA (3 & 4) and their vehicles. The solutions were filtered using a 1.2 μm syringe filter prior infusion, and their pH was measured as well as a titration for calcium quantification was performed. One (1) ml of each solution was administered during 60 minutes for a period of 4 consecutive days. Weight and clinical signs were recorded and observed during the experiment duration.

Experiment 2—1% Ca in ACC. In this experiment 6 SD female rats that were cannulated to their jugular vein were administered with 4 solutions; (1) 1% Ca that were either stabilized with 10% TP (2) 1% Ca that were either stabilized with 20% CA and (3&4) their vehicles. The solutions were filtered using a 1.2 μm syringe filter prior infusion, and their pH was measured as well as a titration for calcium quantification was performed. A dose of 1 ml of each solution was administered during 60 minutes for a period of 3 consecutive days, and then 3 ml of CA-vehicle and 3 ml of 1% Ca-CA solution (after filtration) the inventors administered to rats no. 8 and 11, respectively. Weight and clinical signs were recorded and observed during the experiment duration.

Experiment 3—1% Ca in ACC. In this experiment 5 SD female rats that were cannulated were administered with 4 solutions; 1% Ca that were either stabilized with 20% CA or with 15% CA and their vehicles. The solutions were filtered using a 1.2 μm syringe filter prior infusion, and their pH was measured as well as a titration for calcium quantification was performed. One (1) ml of each solution was administered in a time period of 60 minutes, and then 3 ml of each solution were administered in 60 minutes for 3 consecutive days. Weight and clinical signs were recorded and observed during the experiment duration.

### Results

Hereinafter, is a series of tables summarizing the results of the overall experiment.

TABLE 23
Summary of the second experiment in which animals received 0.3% elemental calcium (before
filtration) in ACC that were either stabilized with 10% TP or 20% CA and their vehicles.

| Animal No. | Treatment | Volume/Time | Duration | Clinical Signs |
|---|---|---|---|---|
| 7 | TP vehicle | 1 ml/60 min | 4 days | Removed catheter |
| | | | | after 4^{th} injection |
| 9 | 0.3% Ca stabilized in 10% TP | 1 ml/60 min | 4 days | Shortness of breath |
| | | | | on the 1^{st} injection |
| 10 | 0.3% Ca stabilized in 10% TP | 1 ml/60 min | 4 days | None |
| 10 | 0.3% Ca stabilized in 10% TP | 1 ml/60 min | 4 days | None |
| 8 | CA vehicle | 1 ml/60 min | 4 days | None |
| 11 | 0.3% Ca stabilized in 20% CA | 1 ml/60 min | 4 days | None |
| 12 | 0.3% Ca stabilized in 20% CA | 1 ml/60 min | 4 days | None |

TABLE 24
Summary of the third experiment in which animals received
1% elemental calcium in ACC (before filtration) that were
either stabilized with 10% TP or 20% CA and their vehicles.

| Animal No. | Treatment | Volume/Time | Duration | Clinical Signs |
|---|---|---|---|---|
| 9 | 1% Ca stabilized in 10% TP | 1 ml/60 min | 3 days | None |
| 10 | TP vehicle | 1 ml/60 min | 3 days | None |
| 8 | CA vehicle | 1 ml/60 min | 3 days | None |
| 11 | 1% Ca stabilized in 20% CA | 1 ml/60 min | 3 days | None |
| 12 | 1% Ca stabilized in 20% CA | 1 ml/60 min | 3 days | None |
| 8 | CA vehicle | 3 ml/60 min | 2 days | None |
| 11 | 1% Ca stabilized in 20% CA | 3 ml/60 min | 2 days | None |

TABLE 25
Summary of the fourth experiment in which animals received 1% elemental calcium in ACC (before
filtration) that was either stabilized with 20% CA or 15% CA and their vehicles.

| Animal No. | Treatment | Volume/Time | Duration | | Clinical Signs |
|---|---|---|---|---|---|
| 18 | 20% CA vehicle | 1 ml/60 min | 1 | day | None |
| 15 | 1% Ca stabilized in 20% CA | 1 ml/60 min | 1 | day | None |
| 17 | 15% CA vehicle | 1 ml/60 min | 1 | day | None |
| 13 | 1% Ca stabilized in 15% CA | 1 ml/60 min | 1 | day | None |
| 16 | 1% Ca stabilized in 15% CA | 1 ml/60 min | 1 | day | None |
| 18 | 20% CA vehicle | 3 ml/60 min | 2 | days | After 2 injections infusion |
| | | | | | could not be given due to |
| | | | | | technical problems |
| 15 | 1% Ca stabilized in 20% CA | 3 ml/60 min | 2 | days | After 2 injections infusion |
| | | | | | could not be given due to |
| | | | | | technical problems |
| 17 | 15% CA vehicle | 3 ml/60 min | 4 | days | None |
| 13 | 1% Ca stabilized in 15% CA | 3 ml/60 min | 4 | days | None |
| 16 | 1% Ca stabilized in 15% CA | 3 ml/60 min | 4 | days | None |

TABLE 26
Animals weight in the beginning of each experiment
and at the end of each experiment.

| Animal No | Initial Weight (gr) | Final Weight (gr) |
|---|---|---|
| 7 | 191 | 200 |
| 9 | 199 | 194 |
| 10 | 191 | 203 |
| 8 | 197 | 203 |
| 11 | 201 | 207 |
| 12 | 199 | 214 |
| 18 | 227 | N/A |
| 15 | 241 | N/A |
| 17 | 210 | N/A |
| 13 | 230 | N/A |
| 16 | 231 | N/A |

TABLE 27
A summary of the solutions used, the calcium concentration after filtration
and calcium carbonate concentration after filtration, and their pH.

| | Calcium concentration | | CaCO₃ | |
|---|---|---|---|---|
| | Before | After | after | |
| Solution | filtration | filtration | filtration | pH |
| Vehicle 10% TP (0.3% Ca) | | 3.32 × 10⁻³% | 8.27 × 10⁻³% | 8.45 |
| 0.3% Ca in 10% TP | 0.3% | 9.55 × 10⁻³% | 0.024% | 10.3 |
| Vehicle 20% CA (0.3% Ca) | | 4 × 10⁻³% | 9.97 × 10⁻³% | 2.47 |
| 0.3% Ca in 20% CA | 0.3% | 0.12% | 0.2925% | 8.16 |
| Vehicle 10% TP (1% Ca) | | 0% | 0% | 8.59 |
| 1% Ca in 10% TP | 1% | 0.021% | 0.051 | 9.83 |
| Vehicle 20% CA (1% Ca) | | 5.24 × 10⁻⁴% | 1.3 × 10⁻³% | 2.18 |
| 1% Ca in 20% CA | 1% | 0.34% | 0.85% | 7.35 |
| Vehicle 20% CA (1% Ca) | | 1.67 × 10⁻³% | 4.16 × 10⁻³% | |
| Vehicle 15% CA (1% Ca) | | 1.42 × 10⁻³% | 3.53 × 10−3% | |
| 1% Ca in 20% CA | 1% | 0.352% | 0.88% | 7.38 |
| 1% Cain 15% CA | 1% | 0.30% | 0.75% | 7.52 |
| 1% Cain 15% CA | 1% | 0.23% | 0.57% | 7.40 |
| 15% CA - Vehicle | | 6.66 × 10⁻⁴% | 16.58 × 10⁻⁴% | 2.20 |
| 0.5% Ca - 10% TP | 0.5% | | | 10.1 |
| 1.5% NaCl | N/A | N/A | N/A | 6.80 |

### Conclusions

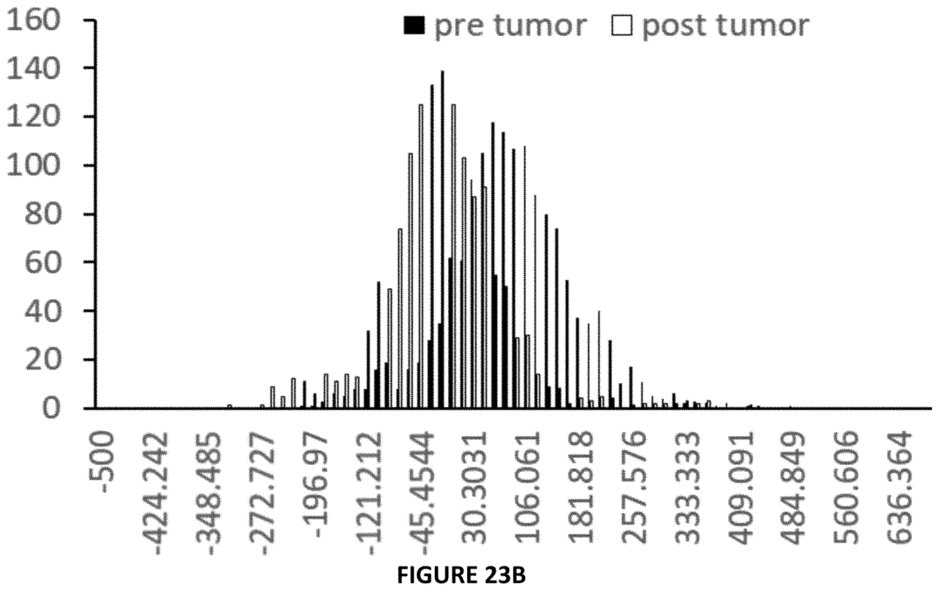

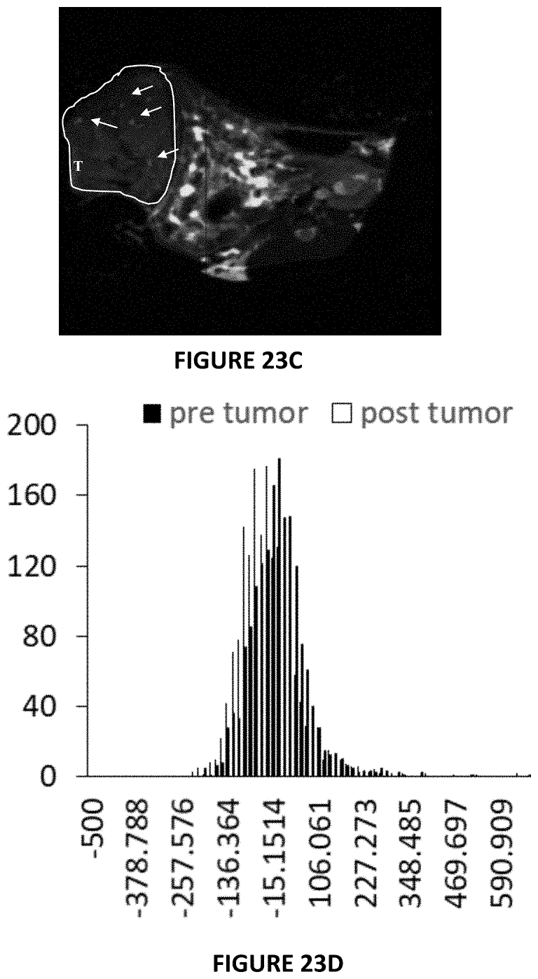

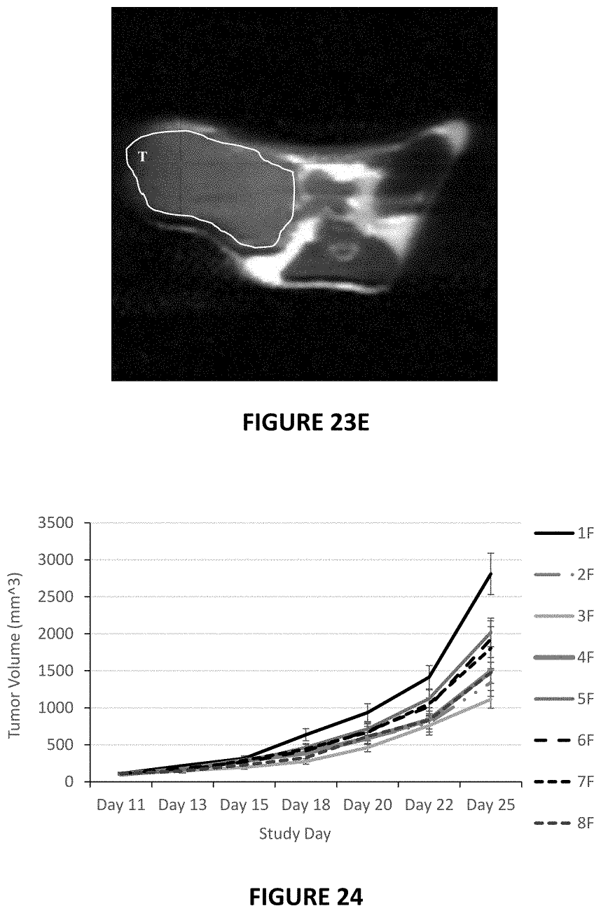

From this set of experiments the inventors observe that there is some morbidity associated with the surgery itself. The inventors analyzed the data presented in Tables 23-27 and observed no clinical signs or toxic effects of the different formulations tested. Only in the first experiment (results showed in FIG. 23) animal no. 9, which received 0.3% calcium in ACC stabilized with 10% TP had experienced shortness of breath after the 1^{st} infusion but not after the other 3 infusions. Another animal (no. 7) removed the catheter. In the second and third experiments the animals showed no signs of distress even though they received higher doses of ACC (Tables 24 and 25). Also, the inventors noticed that in 2 cases the catheters were clogged, and infusion could not be given any more (Table 25). As for animal weights, all animals have gained weight during the experiments (Table 26). The animals from the last experiment did not have their weight measured at the end of the experiment.

An impotent thing the inventors observed in this experiment is that animals showed high tolerability to high doses of ACC. In the Fourth experiment animals received 3 ml of 1% elemental calcium that after filtration was 0.3% and 0.352% calcium, for the 15% CA and 20% CA, respectively according to the titration performed (Table 27). When the inventors calculated the dose according to the animals' initial weights (the animal that received with 20% CA weighed 241 g, and the animals received the 15% CA weighed 230 and 231 g (Tables 25& 26) then these animals received 0.009 gr and 0.01056 gr calcium, respectively for 15% and 20% CA. Which is 0.039 g Ca per 1 Kg for the rats that received 0.3% calcium with 15% CA and 0.0435 gr Ca per 1 Kg for the rat that received 0.352% calcium with 20% CA. If the inventors calculate it for a person weighing 70 Kg then it is 2.73 gr and 3.045 gr, respectively. If the inventors do the same calculations for the calcium carbonate that was measured after filtration (Table 27) then the results are: for the formulation with 15% CA, it was 0.097 g CaCO₃ per 1 Kg which is 6.8 g CaCO₃ for a person weighing 70 Kg and for the 20% CA formulation it was 0.109 g CaCO₃ per 1 Kg which is 7.6 g CaCO₃ for a person weighing 70 Kg. These amounts were given repeatedly with no signs of adverse effects on the animals, showing a NOAEL level of 39-43.5 mg calcium per 1 Kg for 2-4 consecutive days.

In an experiment in which pH was measured consecutively after adding (after filtration) 20% and 15% CA formulation with an initial 1% Ca to medium with 10% bovine serum albumin with a pH of 6.8, the inventors saw that these solutions had elevated the pH to 7.3-7.4 over a period of about 1 hour (FIGS. 2 and 3).

### Example 21

### Effect of ACC on the Immune System of Healthy Mice

In the present study, healthy mice were given ACC for 8 days, whereas the control group received saline also for 8 days. The ACC was given daily as follows: (1) via IP injection—0.2 ml of ACC solution containing 0.1% elemental calcium, and (2) via oral (gavage) administration—ACC 1% w/v (10% TP+1% CA) powder at 5 μm size was used. To 80 mg of ACC powder, water are added to complete to a volume of 8 ml and mixed. A volume of 0.2 ml of this mixture was given to the animals. The spleens of these animals were extracted, and the generated immune cells were analyzed. The results showed that the treated mice had more live leukocytes with higher CD8/CD4 ratio, indicating more cytotoxic T cells within the total population of T cells in the ACC treated animals compared to control. In addition, a lower expression of PD-L1 was found on myeloid cells of the ACC treated animals, compared to the control. These findings indicate that following administration of ACC to healthy animals, the substance mediates a shift towards a better immune system activation.

Additional studies by Amorphical have indicated very active anti-inflammation effects. Many viruses, including the Coronavirus progress into an inflammatory dangerous and fatal pneumonia. Pneumonia may also cause respiratory acidosis due to inefficient removal of CO₂. Amorphical's ACC has already demonstrated anti-inflammatory activities as well healing lung cancer even for patients at hospice stage.

### Conclusion

ACC has been shown to improve and heal animals and humans with inflammations, cancer and other conditions associated with glycolysis and acidosis. Together with a possible effect of activating the immune system, especially increasing the amount of cytotoxic T cells, ACC can potentially reduce symptoms aid the healing when a viral infection occurs.

### Example 22

### Actual Synopsis for a Clinical Trial for ACC as Treatment for COVID-19

This example connects the anti-acidosis characteristics of ACC and the potential for treating COVID-19 patients, due to the involvement of several acidosis effects in the progression of the disease.

The following Synopsis is a pending Clinical Study, Phase 1 for assessing the efficacy of ACC in treating moderate to severe COVID-19 patients. The protocol associated with this synopsis has been already passed internal review boards in 2 unaffiliated hospitals in Israel. It is currently in a process of approval by the Israeli Ministry of Health.

| Study Title | A Phase 1/2; Prospective Randomized Placebo controlled study to | |
|---|---|---|
| | assess the Safety, Efficacy and Tolerability of Amorphous Calcium | |
| | Carbonate (ACC) administered sublingual and in Inhalation | |
| | Concomitantly with BAT as compared to placebo and BAT for the | |
| | treatment of Moderate to Severe COVID-19 patients. | |
| Funder | Amorphical LTD | |
| Clinical Phase | Phase 1/2 | |
| Study Code Name | AMSC-COVID-001 | |
| Study Rationale | Coronavirus disease 2019 (COVID-19) is an infectious disease caused by | |
| | the severe acute respiratory syndrome coronavirus 2 (SARS-CoV-2). The | |
| | disease was first identified in 2019 in Wuhan, China, and has spread | |
| | globally, resulting in the 2019-20 coronavirus pandemic. Common | |
| | symptoms include fever, cough, and shortness of breath. While the | |
| | majority of cases result in mild symptoms, some progress to pneumonia | |
| | and multi-organ failure. The deaths per number of diagnosed cases is | |
| | estimated at between 1% and 5% but varies by age and other health | |
| | conditions. Currently, no antiviral treatment for coronavirus infection has | |
| | been proven to be effective. | |
| | ACC is a safe product that was given in 5 clinical trials to patients with | |
| | various diseases. The proposed treatment is based on the same treatment | |
| | that advanced cancer patients have received and had no severe adverse | |
| | events. | |
| | The ACC has been found to modulate pH's in mild acidic conditions, | |
| | similar to those the SARS-CoV-2 employs in order to fuse into the host | |
| | membrane. This simple, straight forward study might help many patients | |
| | and increase the current available treatments for COVID-19 patients. | |
| Study Objectives | Efficacy Objectives | |
| | Improve severity of disease signs and symptoms as assessed by | |
| | the Disease Severity of Ordinal Scale (Improvement on Ordinal Scale). | |
| Study Design | Study Type: Interventional | |
| | Estimated Enrollment: 100 participants | |
| | Allocation: Randomized Two Arms | |
| | Intervention Model: Placebo Controlled | |
| | Masking: Blinded | |
| | This is a double blind, placebo controlled, randomized study in which ACC | |
| | will be administered concomitantly with BAT vs Placebo and BAT to | |
| | assess the safety, tolerability, and efficacy, in hospitalized patients | |
| | diagnosed with moderate to severe COVID-19. | |
| | This is a multi center study. | |
| | An independent Data and Safety monitoring Committee (DSMC) will | |
| | review continuously the safety as well as the efficacy of the study. | |
| | The study will include two parts: | |
| | Part 1 - a Training period of a single arm active treatment open label, to | |
| | assess the optimal method of study drug administration, as well as the | |
| | safety of the combined administration, on 5 patients. | |
| | Part 2 - a randomized (1:1) placebo controlled 2 arms study. | |
| | The study will include 1:1 randomization (active + BAT vs placebo + BAT) | |
| | Randomization will be stratified by severity of the illness at enrollment: | |
| | Moderate Disease: SpO2 equal or greater than 94%. Respiratory rate equal | |
| | to or less than 25 breath/min | |
| | Severe disease (still do not need mechanical ventilation): SPo2 less than | |
| | 94, Respiratory Rate greater than 25, may need Supplemental Oxygen. | |
| | The following elements will be assessed: | |
| | 1. | Lab Confirmation SARS-CoV-2 infection |
| | 2. | Respiratory Function (breathing rate, SpO₂ Oxygen Saturation |
| | 3. | Vital Signs (BP, HR), Temperature |
| | 4. | Blood Tests: Hematology Biochemistry complete metabolic profile, |
| | | Complete blood count and differential, CRP (C reactive protein), ESR |
| | | (Erythrocytes sedimentation rate), Ferritin, albumin, BUN, creatinine, |
| | | total and free calcium, LDH (total), D dimer. |
| | 5 | Disease Severity Scale (8 points ordinal scale) |
| Subject Population | Inclusion Criteria | |
| key criteria for | 1. | Males and females of age ≥ 18 years and ≤ 80 years |
| Inclusion and | 2. | Signed an Informed Consent |
| Exclusion: | 3. | Agree to undergo blood tests as per protocol |
| | 4. | Diagnosed with COVID-19 |
| | 5. | Evidence of lung involvement (by chest X rays or lung US) |
| | 6. | May or may not need for Supplemental Oxygen at enrollment |
| | 7. | Hospitalized |
| | Exclusion Criteria | |
| | 1 | Pregnant or breast-feeding females |
| | 2. | Patients with non-COVID19 related Pneumonia |
| | 3. | Any pulmonary disease not related to COVID19 |
| | 4. | Tracheostomy |
| | 5. | Mechanical ventilation |
| | 6. | Participating in another clinical study |
| Planned Study | Each patient will be treated at the discretion of the treating physician and | |
| Period | according to the patient's clinical condition (Best Available Symptomatic | |
| | Treatment including Remdesivir) | |
| | The whole study period per patient will be 22 days or until the patient has | |
| | recovered and/or is and/or discharged from the hospital, whichever comes | |
| | first. If a patient is released from Hospital prior to day 22 a follow-up call | |
| | will be done on day 22. | |
| Study Endpoints | Efficacy Endpoints | |
| | Primary Efficacy Endpoints: | |
| | Severity rating of Disease using an 8 point ordinal scale measured on days | |
| | 7, 14, 21. measured as an improvement greater than or equal to 1 point | |
| | from baseline score. | |
| | Ordinal 8 points scale: | |
| | 1. | Not hospitalized and no limitations of activities. |
| | 2. | Not hospitalized, with limitation of activities, home oxygen requirement, |
| | | or both. |
| | 3. | Hospitalized, not requiring supplemental oxygen and no longer requiring |
| | | ingoing medical care (used if hospitalization was extended for infection- |
| | | control or other nonmedical reasons). |
| | 4. | Hospitalized, not requiring supplemental oxygen but requiring ongoing |
| | | medical care (related to Covid-19 or to other medical conditions). |
| | 5. | Hospitalized, requiring any supplemental oxygen. |
| | 6. | Hospitalized, requiring noninvasive ventilation or use of high-flow |
| | | oxygen devices. |
| | 7. | Hospitalized, receiving invasive mechanical ventilation or |
| | | extracorporeal membrane oxygenation (ECMO). |
| | 8. | Death. |
| | Secondary Efficacy Endpoints | |
| | Duration of Hospital stay | |
| | Duration of ICU stay | |
| | Duration of Mechanical Ventilation Use (if needed) | |
| | Duration of Oxygen Supplementation | |
| | Duration of No Oxygen use | |
| | % of patients achieving a score of 3 or lower on the Severity rating of | |
| | Disease scale (Recovery). | |
| | % died | |
| | Safety Endpoints | |
| | 1 | Frequency and severity of adverse events related to study drug, |
| | 2 | Count and percent of patients with hypercalcemia per ACC dose |
| | | and % of patients with hypercalciuria (Urine and blood samples will |
| | | be collected on days 4, 7, 14 and 21) |

| | |
|---|---|
| Statistical Plan | Sample size justification: |
| | This study is a prospective, multicenter, randomized, double blind, placebo |
| | controlled, phase 1/2 study in subjects with COVID-19 illness caused by |
| | SARS-CoV-2. A sample size of 100 subjects with allocation of 50 patients |
| | to the active arm and 50 to the control arm was chosen for this study. Due |
| | to the good safety profile of the product and the unique mechanism of |
| | action are able to proceed with large group size, seen in similar studies in |
| | COVID patients that will consist of 50 patients receiving ACC treatment + |
| | BAT vs. a control group of 50 patients receiving Placebo + BAT, 1:1 |
| | allocation ratio. |
| | Since this is a Phase I/II feasibility study the inventors shall use a 10% level |
| | of significance to determine potential benefit. From data published in the |
| | NEJM November 2020 the % of Covid-19 patients receiving Remdesivir that |
| | improved by 1 point on the Severity scale was 75% versus 66% in the |
| | placebo arm, thus the expected percent in this study ranges between 66% |
| | and 75% for the control arm. With the chosen sample size of 100 patients |
| | 50 per arm, the inventor scan detect a difference of 18%-20% with 80% |
| | power at a 10% level of significance. |
| | Statistical Analyses - general methods |
| | Statistical analyses will be performed using SAS ® v9.4 or higher (SAS |
| | Institute, Cary NC, USA). |
| | Baseline demographic and other baseline characteristics, together with |
| | safety analyses, will be performed on all randomized subjects. Baseline |
| | values are defined as the last valid value prior to treatment. |
| | The required significance level of findings will be 10%, since this a Phase |
| | I/II study. All statistical tests will be two-sided. If statistical tests are |
| | performed, nominal p-values will be presented. Where confidence limits |
| | are appropriate, a two-sided 90% confidence interval will be constructed, |
| | if not specified otherwise. |
| | If not specified otherwise, for comparison of means (continuous variables), |
| | the two-sample t-test or the Wilcoxon rank sum test will be used as |
| | appropriate; and for comparison of proportions (categorical variables), the |
| | Chi-squared test or Fisher's exact test will be used as appropriate. Time to |
| | event data will be presented with Kaplan-Meier curves where two curves |
| | will be compared with a log-rank test. |
| | The safety variable, the cumulative incidence of adverse events (AEs) |
| | observed throughout the study and follow-up period, will be presented in |
| | tabular format. The adverse event rate will be compiled with respect to |
| | frequency, seriousness, causality, and severity of the event, the two groups |
| | will be compared with Fisher's exact test. |
| Experimental and | The investigational ACC product will be composed of the following: |
| Placebo arms | AMOR_inhaled Double Pack- Each kit contains two tubes that after |
| Medicinal Products | mixing result with 1.14% ACC in 10 ml suspension. |
| | AMOR_powder- ACC in a dry powder (up to 2000 mg calcium in |
| | ACC/day sublingually). |
| | Placebo_Inhaled Double Pack - Each kit contains two tubes of saline at |
| | different volumes (similar to investigational product) after mixing the |
| | results remains saline at a final volume of 10 ml. |
| | Placebo_Powder: Each sachet contains powder at the same particle size |
| | and weight as the powder of the investigational product. |
| Data & Safety | Ongoing safety of the subjects will be evaluated by an independent DSMB |
| Monitoring Plan | assigned for this study. |
| Training Period | The first 5 subjects recruited to the study will participate in the training |
| | stage of the study which is meant to assess safety and mode of |
| | administration of ACC administered in both sublingual as well as |
| | inhalation. |
| | At the conclusion of this part a safety review will be done and the |
| | prospective, multicenter, randomized, double blind, placebo controlled, |
| | phase 1/2 study will commence. |
| Biohazard Precautions | The administration of a drug via inhalation poses a biohazard risk on the |
| | medical stuff. Amorphical is aware of that and will take all the necessary |
| | aims to minimize and eliminate this risk. |
| | We will use a disposable device that is intended to deliver in a closed, |
| | filtered system drugs via nebulizers. |
| | The device named Circulaire ® II High-Efficiency Aerosol Drug Delivery |
| | System is FDA approved and is manufactured by Westmed Inc. |
| | https://westmedinc.com/circulaire-ii/ |

### Example 23

### COVID-19 Treatment with ACC—Case Studies

### Case Study I

The following example is a chronicle recorded by a family that several of its members were infected with COVID-19, simultaneously, and they were treated with either ACC powder alone or in combination with inhaled suspension, and sublingually administered.

23 Dec. 2020: Mother-in-law, age 64, did not feel well and had 38.4° C. fever.

24 Dec. 2020: Mother-in-law and 2 daughters were diagnosed positive with COVID-19. Wife feels bad.

26 Dec. 2020: Husband and one of 3 children (age 5) were tested positive too. Wife's conditions deteriorated, she was sent to ER because of low oxygen saturation; released after a few hours with significant symptoms.

27 Dec. 2020: The family received supplies of ACC powder packets aimed for sublingual administration and suspension kits. The kits were taken from the doses prepared for the clinical study, described in Example 21 above. Wife started doing 3 inhalations per day (for 10 min per inhalation) and 6 packets of ACC per day. Husband started taking only 6 packets per day, and all 3 children started taking 3 kids packets with ˜340 mg of ACC each. These doses were taken for 5 days.

28-29 Dec. 2020: Wife's conditions improved significantly as for oxygen saturation and overall feeling. Her loss of smell and taste senses began on December 29, remained for 7 days. She also suffered from muscle aches for 5 days. Husband lost smell and taste senses too, but he regained the senses already after 2 days.

2 Jan. 2021: The other 2 children (ages 2 and 8) were tested and found positive on January third.

4 Jan. 2021: Wife, husband and 5-year-old child are released out of quarantine without any fever or infection symptoms or signs.

10 Jan. 2021: 8-year-old child was released out of quarantine with no fever or infection symptoms or signs. 2-year-old child developed fever for one day and stayed in quarantine until 15 Jan. 2021.

### Case Study II

The following example is a chronicle recorded by a subject that was infected with COVID-19, and was treated with a combination of inhaled suspension of ACC, and sublingual administration of ACC.

A male subject, 67 years old.

Medical Background—experienced a stroke 22 years ago, suffers from diabetes, high blood pressure, high cholesterol levels, problems of liver functions, and a weak immune system. Takes many pills per day. The left side of the body hardly functions.

Disease—On December 31^{st} was diagnosed with COVID-19. Started with treatment at home by Salus company (Prof Uri Rogovsky—medical manager). Was treated with: medications to lower fever, steroids, and blood thinner.

After 11 days with the above treatment, the subject had lost 15 kg of his body weight, did not function at all, stayed at bed all the time, had high fever, and low saturation levels: between 88 to 92.

On January 10 (11 days of disease), ACC administration commenced, as follows: (a) Sublingual administration of 1,500-2,000 mg of powdered ACC/day; and (b) Inhalation of ACC in suspension 3 times per day (morning-noon-evening).

After 4 days of ACC treatment, the saturation level was increased to 97, the patient got off his bed, regained appetite and started to walk and function.

January 25 (26 days after been diagnosed with COVID-19), the subject had recovered and is negative to SARS-CoV-2.

### Case Study III

The following example describes the chronicles of a family that was infected by COVID-19 and treated by ACC administration.

Dec. 23, 2020: The grandmother (mother-in-law, age 64) felt bad, body temperature—38.4° C.

December 24: The daughters (ages 39 and 34) were tested positive to Coronavirus. The husband of the older sister (45) was tested negative.

December 25: 3 children (2, 5, 8) and Husband (45) are tested; Only one child (5) and husband (45) are tested positive.

December 26: The physical conditions of the grandmother (64) is worsening with low oxygen saturation, being checked in the hospital and released to home with overall bad feeling.

December 27: All the family started the ACC treatment for a period of a week (until January 3)
a. Grandmother: Inhalation—3 times a day, each time for 10 minutes; Powder—1,200 mg taken sublingually per day (6 sachets 200 mg each)
b. Husband: Powder—1200 mg sublingually per day (6 sachets 200 mg each)
c. 3 children: Powder—450 mg per day (3 sachets 150 mg each)

December 28-29: A significant improvement of the grandmother's condition, including saturation levels (she also lost taste and smell sense for a week).

December 29-30: Wife and husband lost taste and smell senses (husband for 2 days and wife for 7 days). In addition, the wife suffered from muscle pains for 5 days.

Jan. 2, 2021: Two children (2, 8) are found positive to the virus.

January 4: Grandmother, wife and one child (5) were released from isolation, without any disease's symptoms.

January 10: One child (8) was released from isolation without any disease's symptoms. One child (2) was release on January 15 (due to fever until then).

### Example 24

### In-Vivo pH Evaluation in a Mice Model with Subcutaneous Lewis Lung Carcinoma Treated with ACC

This experiment evaluated the in-vivo pH around a tumor of a Lewis lung carcinoma (LLC) in a mouse treated with ACC compared to a mouse treated with control (saline).

### Background

It is known that cancer cells have a shift in their metabolic pathway towards glycolysis. This shift results with the production of protons and lactate that are secreted into the intercellular environment. It causes the tumor's microenvironment to be acidic. This local acidosis is a driving force for cancer cells proliferation, invasion, metastasis, immune system evasion and drug resistance. Modulation of this local acidosis will lead to a therapeutic effect, such as reduced tumor's growth rate.

### Materials and Methods

Lewis lung carcinoma (LLC) cells (2.5×10⁵ cells in 100 ml ice cold PBS) were intradermal (subcutaneously) injected into the right flank of 2 C57BL/6 female mice, 5- to 7-week-old. Light anesthesia was administered by isoflurane inhalation. Eleven (11) days after cell injection, once tumor reached a measurable size (approximately more than 40 mm³) mice were treated either by in 0.2 ml ACC (0.5% calcium) or 0.2 ml saline via intraperitoneal (IP) injections twice a day, for 14 consecutive days. The entire duration of the study was 25 days. During the experiment, the mice were monitored for any morbidity and mortality. Tumor volumes were measured with a caliper and calculated according to the formula: (length×width²)/2 where length represents the largest tumor diameter, and width represents the smallest tumor diameter.

### CEST/MRI Imaging

At day 25 of the study, after 14 days of treatment mice underwent Chemical exchange saturation transfer (CEST) assay using magnetic resonance imaging (MRI) as described by Longo et al. Iopamidol 61.2% 50 ml INJ IOPAMIRO 300 (Bracco Imaging, Italy) was used as a contrast agent at a dose of 4 gr/kg given intravenously into the mouse's tail vain.

### MRI

MR images were acquired on a 7T scanner (MR Solutions) with a mouse quadrature RF volume coil. Mice were anesthetized with isoflurane vaporized with O₂. Isoflurane was used at 3.0% for induction and at 1.0-2.0% for maintenance. T1 & T2-weighted coronal and axial images were collected for anatomical evaluation. CES′I′ images were acquired with continuous wave RF irradiation (3 T for 5 seconds) by using a single-shot RARE sequence (TR=6 seconds, effective TE=8.7 ms, centric encoding, slice thickens=1.5 mm, FOV=30 mm, matrix=96×96 in-plane spatial resolution=312 m, NA=1) with 46 frequency offsets unevenly distributed from −10 to 10 ppm relative to the water resonance, with the acquisition time for each Z-spectrum being 4 min 36 seconds. The Iopamidol was injected IV into the tail vein at a dose of 4 g 1/kg bodyweight using a tail vain catheter. After waiting 10 minutes, a second CEST sequence was performed with the same parameters. Image analysis was performed using VevoQuant software.

### Results

FIGS. 23A-23E show the pH and anti-cancer effects of IP administering ACC.

FIG. 23A describes tumor volumes measured since treatment began on day 11 of the study. Mice received either ACC or saline injected IP twice a day for 14 consecutive days.

FIGS. 23B-23E show the CEST result before and after contrast agent (Iopamidol) injection a mouse, which was treated with ACC. Black bars in FIG. 23B indicate data acquired prior to injection and white bars represent post injection of contrast agent. FIG. 23C shows the MRI image after injection of the contrast agent.

FIG. 23D shows the CEST result before and after contrast agent (Iopamidol) injection to an infected mouse, which was treated with saline. Black bars indicate data acquired prior injection and white bars indicate post-injection of the contrast agent. FIG. 23D shows the MRI image after injection of the contrast agent.

The MRI image in FIG. 23 above shows tumor growth rates of the 2 mice, which were either treated with ACC or with saline via IP route twice a day for 14 days. It is clear that ACC-treated animals had significantly reduced growth rates compared to the control mice, which received saline. The images in FIGS. 23A-23E demonstrate the pH contrast in the area surrounding the tumors. The tumor border is depicted by the line. For the animal treated with ACC (FIGS. 23B-23C) the tumor area has shown changes towards a basic pH as indicated by the intense red and orange regions (pointed by arrows in FIG. 23C) and as seen by the shift in the CEST sequence graph, done after the injection of Iopamidol (FIG. 23B). Whereas the control animal's image showed lesser red-orange intensity (FIG. 23E), and the CEST sequences are the same before and after injecting the contrast agent, thus indicating the tumor pH as acidic (FIG. 23D).

This experiment showed that ACC administered by IP had caused pH changes in solid LLC tumors.

### Example 25

### Evaluation of Antitumorigenic Effect in Mice Treated with ACC with Different Stabilizers Via Either Intraperitoneal (IP) or Intravenous (IV) Routes

The purpose of this study was to (a) examine the therapeutic effect of different ACC stabilized with citric acid (CA) or triphosphate (TP) and (b) IV versus IP administration on tumor growth rates in a subcutaneous Lewis Lung Carcinoma (LLC) model in mice.

### Materials and Methods

LLC cells in the concentration of (2.6×10⁵ in 100 μl ice cold phosphate buffered saline (PBS) were subcutaneously injected into the right flank of mice C57BL/6 female mice age 5-7 weeks. Once the tumor has reached a volume of >50 mm³, mice were allocated randomly into study groups. The groups and the mode of administration for each are summarized in Table 28.

TABLE 28
The different groups used in this experiment (animal
no.) and the treatment each group received.

| No. Group | | Group name | Dose | | | |
|---|---|---|---|---|---|---|
| (n = x) | (Day “0”) | Treatment | (ml) | | Duration | Route |
| 1 | LLC cancer | Saline | 0.2 | ml | Twice a day | IP |
| (n = 8) | cells in the | | | | 7 days a week | |
| 2 | concentration of | Cisplatin | Per | | Twice a week | IP |
| (n = 8) | 2.6 ×^{105/}cells | | Manufacturing | | | |
| | in 100 μl PBS | | instructions | | | |
| 3 | Inoculated | 0.5% w/v elemental | 0.2 | ml | Twice a day | IP |
| (n = 8) | intradermal | Ca in ACC-TP | | | 7 days a week | |
| | (subcutaneous) | Suspension | | | | |
| 4 | | 0.5% w/v elemental | 0.165 | ml | Once a day | IP |
| (n = 8) | | Ca in ACC-TP | | | 7 days a week | |
| | | Suspension | | | | |
| 5 | | 1% Ca in ACC-CA | 0.36 | ml | Once a day, | IV |
| (n = 16) | | prior filtration. | | | 7 days a week | |
| | | 023% Ca in | | | | |
| | | ACC-CA after | | | | |
| | | filtration | | | | |
| 6 | | 1% Ca in ACC-CA | 0.36 | ml | Once a day | IP |
| (n = 8) | | prior filtration. | | | 7 days a week | |
| | | 023% Ca in | | | | |
| | | ACC-CA after | | | | |
| | | filtration | | | | |
| 7 | | 1% Ca in ACC-CA | 0.36 | ml | Twice a day, | IP |
| (n = 8) | | prior filtration. | | | 7 days a week | |
| | | 023% Ca in | | | | |
| | | ACC-CA after | | | | |
| | | filtration | | | | |

| | | | | |
|---|---|---|---|---|
| 8 | 0.5% w/v elemental | 0.2 | Twice a day, | IP |
| (n = 8) | Ca in ACC-CA | | 7 days a week | |
| | Suspension | | | |

### Preparation of Solutions.

1. Preparation of Saline solution—0.9 g of NaCl dissolved in 80 mL DDW, Complete with DDW to a volume of 100 mL.

2. Preparation of the ACC Suspension 0.5% w/v Ca (Stabilized by 100% TP) by 4 steps: The following stock solutions of each ingredient was prepared and filtered in a biological hood: (1) Calcium Chloride Dihydrate (CaCl₂.2H₂O)—Volume 0.04 L in 0.020 L DDW, weigh 1.84 g CaCl₂.2H₂O Complete with DDW to a volume of 0.04 L; (2) Sodium TriPolyPhosphate (STPP or TP)—Volume 0.02 L in 0.01 L DDW, weigh 0.184 g TP. Complete with DDW to a volume of 0.02 L; and (3) Sodium Carbonate—Na₂CO₃—Volume 0.04 L in 0.02 L DDW, weigh 1.33 gr Na₂CO₃. Complete with DDW to a volume of 0.04 L.

In order to prepare a 10 ml suspension of ACC stabilized with 10% TP, the above stock solutions were mixed according to the table below. Each component was added according to the following table's sequence at once. The solutions were well shaken for at least 30 seconds until uniform and stable suspensions are obtained.

| Solution | (ml) |
|---|---|
| 1- Calcium Chloride Dihydrate | 4 |
| 2- TP | 1 |
| 3- Sodium Carbonate | 4 |
| 2- TP | 1 |

3. Preparation of the ACC Suspension 1% w/v Ca (Stabilized by 15% CA) by 4 steps: The following stock solutions for each ingredient were prepared and filtered in a biological hood: (1) Calcium Chloride Dihydrate (CaCl₂.2H₂O)—Volume 0.04 L in 0.020 L DDW, weigh 3.68 g CaCl₂.2H₂O. Complete with DDW to a volume of 0.04 L; (2) Sodium Carbonate—Na₂CO₃—Volume 0.04 L in 0.02 L DDW, weigh 2.65 g Na₂CO₃ Complete with DDW to a volume of 0.04 L; and (3) Citric Acid (CA)—Volume 0.02 L in 0.01 L DDW, weigh 0.55 g CA. Complete with DDW to a volume of 0.02 L.

In order to prepare a 10 ml solution of ACC stabilized with 10% TP, the above stock solutions should be mixed according to the table below. Each component needs to be rapidly added at once. The solutions were well shaken for at least 30 second until uniform and stable suspension is obtained. After the suspensions were prepared, they were filtered using a 1.2 μm syringe filter prior injection into the animals.

| Solution | (ml) |
|---|---|
| 1- Calcium Chloride Dihydrate | 4 |
| 2- CA | 1 |
| 3- Sodium Carbonate | 4 |
| 2- CA | 1 |

4. Preparation of the ACC Suspension 0.5% w/v Ca (Stabilized by 15% CA) by 4 steps: The following stock solutions of each ingredient were prepared and filtered in a biological hood: (1) Calcium Chloride Dihydrate (CaCl₂.2H₂O)—Volume 0.08 L. in 0.02 L DDW, weigh 3.68 g CaCl₂.2H₂O. Complete with DDW to a volume of 0.08 L; (2) Sodium Carbonate—Na₂CO₃—Volume 0.08 L. In 0.02 L DDW, weigh 2.65 g Na₂CO₃ Complete with DDW to a volume of 0.08 L; and (3) Citric Acid (CA)—Volume 0.04 L in 0.01 L DDW, weigh 0.55 g CA. Complete with DDW to a volume of 0.02 L.

In order to prepare a 10 ml solution of ACC stabilized with 10% TP, the above stock solutions were mixed according to the sequence of the table below. Each component was added at once. The suspension was mixed well for at least 30 second until uniform stable suspension was obtained.

| Solution | (ml) |
|---|---|
| 1- Calcium Chloride Dihydrate | 4 |
| 2- CA | 1 |
| 3- Sodium Carbonate | 4 |
| 2- CA | 1 |

### Results

### Tumor Size Determination

Tumor volumes were measured every other day with a caliper and calculated according to the formula: (length×width²)/2 where length represents the largest tumor diameter, and width represents the smallest tumor diameter. The tumor growth in all the model groups are illustrated in FIG. 24 and summarized in Table 29. The table describes the treatments the different groups received, routes of administration, doses and average (mean) tumors volumes, standard error mean (SEM) on Day 25. t test results comparing each group against the saline are presented. Notice that P<0.05 in all treatments compared to the negative control (saline).

TABLE 29
The effects of various treatments on Day 25.

| Group | 1F | 2F | 3F | 4F | 5F | 6F | 7F | 8F |
|---|---|---|---|---|---|---|---|---|
| Treatment | Saline | Cisplatin | ACC- | ACC- | ACC- | ACC- | ACC- | ACC- |
| | | | TP | TP | CA | CA | CA | CA |
| % calcium | N/A | N/A | 0.5 | 0.5 | 0.23 | 0.23 | 0.23 | 0.5 |
| given | | | | | | | | |
| Dose | 0.2mlx | Per | 0.2mlx | 0.165 ml | 0.36 ml | 0.36 ml | 0.36 ml | 0.2mlx |
| | 2/d | manu- | 2/d | x1/d | x1/d | x1/d | x2/d | 2/d |
| | | facturer | | | | | | |
| Dose Ca | N/A | N/A | 100 | 45.25 | 41.4 | 41.4 | 82.8 | 100 |
| mg/kg/day | | | | | | | | |
| Admini- | IP | IP | IP | IP | IV | IP | IP | IP |
| stration | | | | | | | | |
| route | | | | | | | | |
| Mean | 2808 | 1342 | 1113 | 1491 | 2019 | 1926 | 1802 | 1472 |
| (mm³) | | | | | | | | |
| SEM | 279 | 188 | 118 | 272 | 193 | 248 | 294 | 144 |
| t test | | 0.0007 | 0 | 0.01 | 0.03 | 0.04 | 0.012 | 0.0006 |

This experiment evaluated whether ACC stabilized with TP or CA had an anti-cancer effect on tumors growth rates and assess the differences in IP vs IV administration.

In order to develop more systemic routes for administration it is important to develop an ACC solution with lower basic pH while still maintaining the modulation effect over acidic environments and adequate stability of the suspensions against crystallization. Therefore, the use of citric acid (CA) instead of TP was considered, which is an acceptable compound for IV administrations. When ACC was stabilized with TP the pH of the solution is between 9 and 10. However, when ACC was stabilized with CA the pH range was dropped to between 7.2 and 7.8. This is a more acceptable pH range for IV administration.

As seen in FIG. 24, starting from Day 18 of the experiment (day 8 of treatment) the treatment groups started to differ from the saline group. These differences in growth rates became larger as the experiment and treatments proceeded.

As can be seen in Table 29 all groups differed from the negative control group in a statistically significant manner. It should be mentioned that a t test comparing the tumor volumes of the group treated with ACC-CA given IV (5F) to the ACC-TP treated group (3F) differed in a non-statistically significant manner, although there were differences between these groups on the tumor growth rates, with preference to the ACC-TP. Also, the ACC-CA formulations that underwent filtration (groups 5F, 6F &7F) resulted with a similar effect on the growth rate of the tumors, which was lesser than the ACC-CA that was not filtered and had a calcium concentration of 0.5% (group 8F). This group (8F) had similar results to the parallel ACC-TP group (3F) and the one with a lower dose due to lower volume of administration (0.165 ml) (Group 4F). Comparison between ACC-CA that was administered either by IV (5F) or IP (6F) gave very similar results, suggesting that both types of administrations are effective and feasible.

To summarize, it can be concluded that ACC stabilized with CA and TP both had an anticarcinogenic effect as seen by the decrease of tumors growth rates.

### Example 26

### Inflammation Treatment of Various Patients with ACC

Table 30, herein below, summarizes numerous cases of patients that suffered from various inflammations for prolonged periods of time before taking various doses and modes of administration of ACC, as described herein. The table records the disappearance or lessening of the different inflammation symptoms and the durations until such a relief was evident.

| | | | | | ACC | |
|---|---|---|---|---|---|---|
| | | | | How | Dosage | |
| # | Gender | Age | Prognosis | long | per day | Improvement |
| 1 | Male | 79 | Knees’ pain and | several | 400 mg | pain relief in |
| | | | inflammations | years | | two weeks, |
| | | | | | | can stand up |
| | | | | | | easily from |
| | | | | | | sitting |
| | | | | | | position |
| 2 | Male | 25 | Inflammations and | — | 400 mg - 2 | pain relief |
| | | | muscles pain after | | sachets of | after two days |
| | | | workouts | | Density | |
| | | | | | Sport per | |
| | | | | | day | |
| 3 | Female | 76 | Wrists pain and | over 3 | 400 mg | Improvement |
| | | | inflammations in | years | | felt after some |
| | | | feet | | | months |
| 4 | Male | 50 | DOMS (Delayed | — | 1 sachet | lessen effects |
| | | | Onset Muscle | | of Density | of DOMS and |
| | | | Soreness) following | | Sport | improves |
| | | | physical practice | | prior to | physical |
| | | | | | running, | performance |
| | | | | | sometimes | |
| | | | | | a sachet | |
| | | | | | after it | |
| | | | | | (200-400 | |
| | | | | | mg) | |
| | Female | 70 | Arthritis, abrasion | 15 | 800 mg | No more |
| | | | of spine, wrists and | years | | Inflammations |
| | | | shoulders. In | | | and pain |
| | | | wheelchair | | | |
| 6 | Female | 63 | Calcifications in | 5 | 400-600 | Better |
| | | | knees, kidneys, | years | mg | absorption of |
| | | | shoulders and | | | calcium- as |
| | | | thyroid gland | | | seen in |
| | | | | | | urinalysis |
| 7 | Female | | Inflammations and | — | 1200 mg | No more |
| | | | nad pain in knees | | | Inflammations |
| | | | | | | and pain |
| 8 | Female | 65 | Hypopara- | 9 | 800 mg | better |
| | | | thyroidism + | years | | absorption of |
| | | | hypothyroidism | | | calcium, was |
| | | | | | | part of the |
| | | | | | | trial in |
| | | | | | | Rambam |
| | | | | | | hospital |
| 9 | Female | 25 | Parathyroid gland | 2 | 1600 mg | high level of |
| | | | was damaged after | years | | calcium |
| | | | removal of thyroid | | | absorption |
| | | | gland | | | and the |
| | | | | | | tingling was |
| | | | | | | gone |
| 10 | Male | 52 | Knees’ pains and | — | 800-1200 | In 2-3 |
| | | | inflammation | | mg | months, relief |
| | | | | | | of pain and |
| | | | | | | fast recovery |
| | | | | | | after sport. |
| | | | | | | Also, after hip |
| | | | | | | replacement - |
| | | | | | | the recovery |
| | | | | | | rate was |
| | | | | | | exceptionally |
| | | | | | | fast (3 weeks) |
| 11 | Male | 62 | Pain in hip and | — | 800 mg | After 2 |
| | | | inflammations in | | | weeks, the |
| | | | wrists | | | pain had |
| | | | | | | reduced |
| | | | | | | gradually |
| 12 | Female | 62 | Arthritis and severe | — | 800 mg | Pain gone |
| | | | osteoporosis, pain | | | after few |
| | | | of wrists and knees | | | weeks of |
| | | | | | | usage |

1. A method for treating a subject afflicted with an acidosis-related disease or condition, comprising orally administering to said subject a therapeutically effective amount of a solid composition comprising amorphous calcium carbonate (ACC) particles stabilized by at least one stabilizing agent, wherein said solid composition of ACC particles is formulated for controlled release.

2. The method of claim 1, wherein said ACC particles are agglomerated particles, optionally wherein said composition further comprises an enteric coating, and optionally wherein said composition comprising ACC particles is coated or encapsulated within said enteric coating.

3.-4. (canceled)

5. A method for treating a subject afflicted with an acidosis-related disease or condition, comprising administering to said subject a therapeutically effective amount of an aqueous composition in the form of a dispersion or suspension comprising ACC particles stabilized by at least one stabilizing agent, wherein said ACC particles are substantially uniformly dispersed or suspended in said composition, and wherein said administering is injecting, optionally wherein said injecting comprises: intravenously injecting, intraperitoneal injecting, locally injecting, or any combination thereof.

6. (canceled)

7. The method of claim 1, further comprising diagnosing said acidosis-related disease or condition in said subject prior to said administering, and optionally wherein: (i) acidosis-related disease or condition is selected from the group consisting of: inflammation or a disease or condition associated therewith, prostate cancer, colorectal cancer, non-small cell lung cancer (NSCLC), human epidermal growth factor receptor (HER) positive breast cancer, and any combination thereof; (ii) said inflammation or a disease or condition associated therewith is related or derived from a physical activity; (iii) wherein said inflammation or a disease or condition associated therewith that is related or derived from a physical activity comprises: hip stress fracture, inflammation of the adductor magnus, swelling, redness and local warmness of the knee, or any combination thereof; (iv) wherein said acidosis-related disease or condition is selected from the group consisting of: rheumatoid arthritis, diabetes mellitus, arteritis, osteoarthritis, hyperlactatemia, renal tubular acidosis, an infectious disease, ventilatory failure, sepsis, anaerobic and aerobic exercise, and any combination thereof; (v) wherein said acidosis-related disease or condition is rheumatoid arthritis; (vi) wherein said administering is intraperitoneally injecting; (vii) wherein said infectious disease is induced by a virus; (viii) wherein said infectious disease is a respiratory disease; (ix) said virus belongs to a family selected from the group consisting of: Coronaviridae, Filoviridae, Arenaviridae, Orthomyxoviridae, Paramyxoviridae, Retroviridae, Togaviridae, and Flaviviridae; (x) wherein said infectious disease is induced by a coronavirus; (xi) said infectious disease is Coronavirus disease 2019 (COVID-2019); (xii) said acidosis-related disease or condition involves acidophilic Cathepsin activity: (xiii) said acidophilic Cathepsin is selected from the group consisting of: B, K, A, G, C, F, H, L, O, V, W, X, D, E, and any combination thereof; (xiv) said acidophilic Cathepsin is Cathepsin B, Cathepsin K, or both; or (xv) said treating comprises reducing activity of said acidophilic Cathepsin in said subject.

8.-22. (canceled)

23. A method for treating a subject afflicted with inflammation or a disease or a condition associated therewith, the method comprising administering to said subject a therapeutically effective amount of: (i) a solid composition of ACC stabilized by at least one stabilizing agent; (ii) an aqueous composition in the form of a dispersion or suspension of ACC particles stabilized by at least one stabilizing agent; or (iii) a combination of (i) and (ii), thereby treating the subject afflicted with inflammation or a disease or a condition associated therewith.

24. The method of claim 23, wherein said disease comprises an infectious disease.

25. The method of claim 23, wherein said infectious disease comprises a viral infectious disease.

26. The method of claim 24, wherein said infectious disease is COVID-2019.

27. The method of claim 26, wherein said administering comprises administering by inhalation, sublingual administering, or both.

28. The method of claim 26, wherein said administering comprises multiple administrations, optionally wherein said multiple administering comprises daily multiple administering.

29. (canceled)

30. The method of claim 27, wherein said administration by inhalation comprises administering said aqueous composition in the form of a dispersion or suspension comprising said ACC particles stabilized by at least one stabilizing agent in a wt % ranging from 1 wt % to 2.0 wt % of said dispersion or suspension.

31. The method of claim 27, wherein said sublingual administration comprises administering said solid composition of ACC particles stabilized by at least one stabilizing agent comprising calcium in an amount ranging from 1,000 to 2,500 mg calcium per day, in the form of ACC.

32. The method of claim 23, wherein said solid composition of ACC stabilized by at least one stabilizing agent is formulated for controlled release, optionally wherein said solid composition comprises an enteric coating, and optionally wherein said solid composition of ACC is coated or encapsulated within an enteric coating.

33.-34. (canceled)

35. The method of claim 23, wherein at least 30% of said ACC particles comprise primary particles having a maximal size ranging from 10 to 500 nm.

36. The method of claim 23, wherein said ACC is substantially soluble in pH ranging from 6.0 to 7.5.

37. The method of claim 23, wherein said at least one stabilizing agent is selected from the group consisting of: organic acids, phosphorylated, phosphonated, sulfated or sulfonated organic compound, phosphoric or sulfuric esters and ethers of hydroxy carboxylic acids and polyols, glucose and its derivatives, polysaccharides, phosphorylated amino acids, bisphosphonates, polyphosphonates, organic polyphosphates, inorganic polyphosphates, hydroxyl bearing organic compounds and polyols, proteins, salt and derivatives thereof, magnesium or a salt thereof, and any combinations thereof.

38. The method of claim 23, wherein said composition further comprises an additional biomedically active agent.

39. The method of claim 38, wherein said additional biomedically active agent is suitable for the treatment or prevention of an acidosis-related disease or condition.

40. The method of claim 38, wherein said additional active agent is hyaluronic acid.

41. The method of claim 23, wherein said composition is a nutraceutical composition or a pharmaceutical composition, optionally said nutraceutical composition comprises a food supplement or a medical food.

42.-45. (canceled)
